(19) United States
(12) Patent Application Publication
(10) Pub. No.: US 2006/0223815 A1
(43) Pub. Date: Oct. 5, 2006

(54) Therapeutic agents comprising an anti-angiogenic agent in combination with an src-inhibitor and their therapeutic use

(76) Inventors: Jon Owen Curwen, Cheshire (GB); Stephen Robert Wedge, Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 10/555,389

(22) PCT Filed: May 4, 2004
(86) PCT No.: PCT/GB04/01939

§ 371 Date: Nov. 3, 2005

(30) Foreign Application Priority Data
May 7, 2003 (GB) 0310401.5

(51) Int. Cl. A61K 31/517

(52) U.S. Cl. 514/252.17; 514/266.2; 514/266.22

(57) ABSTRACT

The invention relates to the use of an anti-angiogenic agent in combination with an inhibitor of the Src family of non-receptor tyrosine kinases in the manufacture of a medicament for use in the substantially normotensive treatment in a warm-blooded mammal such as a human being of a disease state associated with angiogenesis, the Src kinase inhibitor being administered in an amount effective to counteract substantially the hypertension induced by the anti-angiogenic agent.

The present invention relates to the use of an anti-angiogenic agent in combination with an inhibitor of the Src family of non-receptor tyrosine kinases in the manufacture of a medicament for use in the production of an anti-angiogenic and/or an anti-cancer effect, to a method for providing an anti-angiogenic and/or an anti-cancer effect by the administration of an anti-angiogenic agent and an inhibitor of the Src family of non-receptor tyrosine kinases, to a combination product comprising a particular anti-angiogenic agent and a particular inhibitor of the Src family of non-receptor tyrosine kinases and to a pharmaceutical composition comprising a particular anti-angiogenic agent and a particular inhibitor of the Src family of non-receptor tyrosine kinases. In particular, the present invention relates to the use in combination of an anti-angiogenic agent that is an inhibitor of the vascular endothelial growth factor (hereinafter VEGF) receptor tyrosine kinases together with an inhibitor of the Src family of non-receptor tyrosine kinases. The invention is useful in a method for the treatment of diseases associated with angiogenesis and in a method for the treatment or prophylaxis of cancer, particularly of solid tumour disease.

Current options for treating cancer include surgical resection, external beam radiation therapy and/or systemic chemotherapy. These are partially successful in some forms of cancer but are less successful in others. There is a continuing need for new therapeutic treatments for treating cancer.

Inhibition of VEGF Receptor Tyrosine Kinases

Normally, angiogenesis, the process of forming new blood vessels, plays an important role in a variety of processes including embryonic development, wound healing and several components of female reproductive function. However, undesirable or pathological angiogenesis has been associated with a number of disease states including diabetic retinopathy, psoriasis, cancer, rheumatoid arthritis, atheroma, Kaposi's sarcoma and haemangioma (Fan et al., Trends in Pharmacol. Science, 1995, 16, 57-66; Folkman, Nature Medicine, 1995, 1, 27-31).

Angiogenesis is stimulated via the promotion of the growth of endothelial cells. Several polypeptides with in vitro endothelial cell growth promoting activity have been identified including acidic and basic fibroblast growth factors (aFGF and bFGF) and VEGF. By virtue of the restricted expression of its receptors, the growth factor activity of VEGF, in contrast to that of aFGF and bFGF, is relatively specific towards endothelial cells. Recent evidence indicates that VEGF is an important stimulator of both normal and pathological angiogenesis (Jakeman et al., Endocrinology, 1993, 133, 848-859; Kolch et al., Breast Cancer Research and Treatment, 1995, 36, 139-155) and vascular permeability (Connolly et al., J. Biol. Chem., 1989, 264, 20017-20024). Alteration of vascular permeability is also thought to play a role in both normal and pathological physiological processes (Senger et al., Cancer and Metastasis Reviews, 1993, 12, 303-324).

Receptor tyrosine kinases (RTKs) are important in the transmission of biochemical signals across the plasma membrane of cells. These transmembrane molecules characteristically consist of an extracellular ligand-binding domain connected through a segment in the plasma membrane to an intracellular tyrosine kinase domain. Binding of ligand to the receptor results in stimulation of the receptor-associated tyrosine kinase activity which leads to phosphorylation of tyrosine residues on both the receptor and other intracellular molecules. These changes in tyrosine phosphorylation initiate a signalling cascade leading to a variety of cellular responses. To date, a number of distinct RTK subfamilies, defined by amino acid sequence homology, have been identified. One RTK family comprises the fms-like tyrosine kinase receptor Flt-1, the kinase insert domain-containing receptor KDR (also referred to as Flk-1) and the fms-like tyrosine kinase receptor Flt-4. Two of these related RTKs, namely Flt-1 and KDR, have been shown to bind VEGF with high affinity (De-Vries et al., Science, 1992, 255, 989-991; Terman et al., Biochem. Biophys. Res. Comm., 1992, 187, 1579-1586). Binding of VEGF to these receptors expressed in heterologous cells has been associated with changes in the tyrosine phosphorylation status of cellular proteins and calcium fluxes.

VEGF is a key stimulus for vasculogenesis and angiogenesis. This cytokine induces a vascular sprouting phenotype by inducing endothelial cell proliferation, protease expression and migration, and subsequent organisation of cells to form a capillary tube promoting formation of a hyper-permeable, immature vascular network which is characteristic of pathological angiogenesis. It has been shown that activation of KDR alone is sufficient to promote all of the major phenotypic responses to VEGF, including endothelial cell proliferation, migration and survival, and the induction of vascular permeability.

Accordingly, antagonism of the activity of VEGF is expected to be beneficial in the treatment of a number of disease states that are associated with angiogenesis and/or increased vascular permeability such as cancer, especially in inhibiting the development of tumours.

Src Non-Receptor Tyrosine Kinase Inhibition

In recent years it has been discovered that cells may become cancerous by virtue of the transformation of a portion of its DNA into an oncogene i.e. a gene which, on activation, leads to the formation of malignant tumour cells. It is known, for example, that several oncogenes encode tyrosine kinase enzymes and that certain growth factor receptors are also tyrosine kinase enzymes. The first group of tyrosine kinases to be identified arose from such viral oncogenes, for example pp60^{v-Src} tyrosine kinase (otherwise known as v-Src) and the corresponding tyrosine kinases in normal cells, for example pp60^{c-Src} tyrosine kinase (otherwise known as c-Src).

The Src family of non-receptor tyrosine kinases is located intracellularly and is involved in the transmission of biochemical signals such as those that influence tumour cell motility, dissemination and invasiveness and subsequently metastatic tumour growth. Members of the Src family include inter alia c-Src, c-Yes, c-Plck and c-Fyn.

It is further known that the Src family of non-receptor tyrosine kinases is highly regulated in normal cells such that, in the absence of extracellular stimuli, the kinases are maintained in an inactive conformation. However, some Src family members, for example c-Src tyrosine kinase, are frequently significantly activated (when compared to normal cell levels) in common human cancers.

Accordingly it has been recognised that an inhibitor of such non-receptor tyrosine kinases should be of value as a selective inhibitor of the motility of tumour cells and as a selective inhibitor of the dissemination and invasiveness of mammalian cancer cells leading to inhibition of metastatic tumour growth. Thus the predominant role of c-Src non-receptor tyrosine kinase is to regulate cell motility which is necessarily required for a localised tumour to progress through the stages of dissemination into the blood stream, invasion of other tissues and initiation of metastatic tumour growth. c-Src kinase is involved in the signal transduction steps which lead to the invasiveness and migratory ability of metastasising tumour cells.

Accordingly Src linase inhibitors are of value as anti-tumour agents, in particular as selective inhibitors of the motility, dissemination and invasiveness of mammalian cancer cells leading to inhibition of metastatic tumour growth. Particularly, Src kinase inhibitors are of value as anti-invasive agents in the containment and/or treatment of solid tumour disease. Particularly, such compounds are expected to be useful in the prevention or treatment of those tumours which are sensitive to inhibition of one or more of the multiple non-receptor tyrosine kinases such as c-Src kinase that are involved in the signal transduction steps which lead to the invasiveness and migratory ability of metastasising tumour cells. Further, such compounds are expected to be useful in the prevention or treatment of those tumours which are mediated alone or in part by inhibition of the enzyme c-Src, i.e. the compounds may be used to produce a c-Src enzyme inhibitory effect in a warm-blooded animal in need of such treatment. Specifically, such compounds are expected to be useful in the prevention or treatment of solid tumour disease.

Linkage of Growth Factor Receptors to Blood Pressure Effects

A complex interaction of a number of mediators leads to the strict control of blood pressure in the normal mammal. The system is such that if the level of one mediator changes the resultant effect is compensated for by the other mediators such that normal blood pressure is maintained (Guyton et al., Annual Review of Physiology, 1972, 34, 13-46, and Quan et al., Pacing and Clinical Electrophysiology, 1997, 20, 764-774). It is important that blood pressure is tightly controlled because hypertension underlies a variety of cardiovascular diseases such as stroke, acute myocardial infarction and renal failure.

Many substances exhibit effects on blood vessels in vitro which, in isolation, would suggest effects on blood pressure in vivo. However, because of the nature of the compensation mechanisms that control blood pressure, it is often the case that anticipated in vivo effects are not obtained and thus normal blood pressure is maintained. It has been reported that various growth factor receptors may be involved as mediators in the control of blood pressure in the normal mammal.

(a) Blood Pressure Effects of VEGF Receptor Tyrosine Kinases

It has been reported that VEGF and FGP have acute effects on vascular tone. VEGF has been shown to dilate dog coronary arteries in vitro (Ku et al., Amer. J. Physiology, 1993, 265, H585-H592) and to induce hypotension in the conscious rat (Yang et al., J. Cardiovascular Pharmacology, 1996, 27, 838-844). However, in vivo the effects of these agents are only transitory. Even with a very large dose of VEGF (250 μg/kg) in conscious rats, Yang et al. observed a return to normal blood pressure within 20 minutes. At lower doses of VEGF, blood pressure returned to normal significantly faster. A similar effect was observed in anaesthetised rats with the blood pressure returning to normal within 30 minutes of the administration of 15 μg/kg bFGF (Boussairi et al., J. Cardiovascular Pharmacology, 1994, 23, 99-102). These studies also showed that tachyphylaxis (or desensitisation) quickly develops following growth factor administration. Thus, further administration of growth factor has no effect on blood pressure.

It has been reported that the vasodilation induced by both FGF and VEGF depends, at least in part, on the release of nitric oxide (Morbidelli et al., Amer. J. Physiology, 1996, 270 H411-H415 and Wu et al., Amer. J. Physiology, 1996, 271, H1087-H1093).

The complexity and confusion as to the effect of VEGF on blood pressure is illustrated by the following two patent applications that disclose contrasting effects.

A method for treating a hypertensive disorder in a pregnant woman is described in International Patent Application WO 98/28006, the method comprising administering an amount of a therapeutic substance which regulates the amount and/or activity of VEGF. Thus, according to this disclosure, a VEGF RTK inhibitor may be expected to reduce blood pressure.

However, a method for treating essential hypertension is described in International Patent Application WO 00/13703, the method comprising administering to a patient an effective amount of an angiogenic factor such as VEGF, or an agonist thereof. Thus, according to this disclosure, a VEGF RTK inhibitor may be expected to increase blood pressure.

More recently, it has been disclosed in International Patent Application WO 01/74360 that VEGF receptor tyrosine kinase inhibitors, provided that they possess suitable pharmacokinetic properties which provide reasonable bioavailability, do lead to a sustained increase in blood pressure when administered to rats, particularly when administered chronically.

(b) Blood Pressure Effects of Src Non-Receptor Tyrosine Kinase

As with the initial studies of the effect of VEGF on blood pressure, there is complexity and confusion as to the effect of Src kinase on blood pressure as illustrated by the following two groups of disclosures.

On the one hand, it has been disclosed in various papers concerning the in vitro electrophysiologic effects of tyrosine kinases including c-Src kinase that tyrosine kinase enzymatic activity can be involved in the movement of calcium ions across cellular membranes (Wijetunge et al., Biochem. Biophys. Res. Comm., 1992, 189, 1620-1623, Biochem. Biophys. Res. Comm., 1995, 217, 1039-1044 and British Journal of Pharmacology, 1998, 124, 307-316 and Hu et al., Journal of Biological Chemistry, 1998, 273, 5337-5342). However, there does not appear to have been any disclosure of the relevance of such in vitro effects of Src kinase on blood pressure control in vivo in a warm-blooded animal such as man.

In contrast, it has been disclosed in International Patent Application WO 99/61590 that Src kinase may be used to modulate the angiogenesis in tissues caused by ‘angiogenic molecules’ such as bFGF. As discussed hereinbefore, VEGF is another ‘angiogenic molecule’. In addition, it has been disclosed by Cheresh et al., in Nature Medicine, 2001, 7, 222-227, and International Patent Application WO 01/45751, that the angiogenesis factor VEGF is produced in response to ischaemic injury, for example cerebral ischaemia (stroke) in the brain. It was disclosed that VEGF alone did not cause an increase in vascular permeability leading to brain oedema and tissue damage but that Src kinase activity regulates (i.e. controls) the ability of VEGF to increase vascular permeability and that a Src kinase inhibitor could block vascular permeability. Using animal studies, it was disclosed that the administration of the Src inhibitor PP1 reduced infarct volume following cerebral ischaemia and that there was no direct effect on cerebral blood flow. It was asserted that Src kinase inhibition may be useful to prevent secondary damage following a stroke and may also ‘impact the course of other ischemic diseases such as myocardial infarction’.

If Src kinase activity does control the effectiveness of VEGF, it might be reasonable to expect that a Src kinase inhibitor, when administered chronically, would have a similar effect on blood pressure as a VEGFR tyrosine kinase inhibitor i.e. a hypertensive effect (as disclosed in International Patent Application WO 01/74360).

However, more recently, it is described in co-pending United Kingdom Patent Application No. 0307333.5 that Src kinase inhibitors do cause a decrease in blood pressure. In particular, a selective Src kinase inhibitor causes a substantial decrease in blood pressure. More particularly, a selective Src kinase inhibitor that possesses pharmacokinetic properties which provide a reasonable bioavailability when administered chronically to a warm-blooded animal causes a sustained decrease in blood pressure.

Disclosures of the Combiniation of a VEGF Receptor Kinase Inhibitor and a Src Kinase Inhibitor

It is disclosed in International Patent Applications WO 97/22596, WO 98/13354 and WO 01/32651 that the anti-angiogenic and/or vascular permeability reducing compounds defined therein may be administered as a sole therapy or in conjunction with surgery, radiotherapy or chemotherapy. The listed chemotherapy options included anti-invasion agents (for example metalloproteinase inhibitors like marimastat and inhibitors of urokinase plasminogen activator receptor function) and inhibitors of growth factor function (for example platelet derived growth factor and hepatocyte growth factor, growth factor antibodies, growth factor receptor antibodies, tyrosine kinase inhibitors and serine/threonine kinase inhibitors).

Further, it is disclosed in International Patent Applications WO 01/94341 and WO 02/16352 that a Src kinase inhibitor may be used to provide an anti-invasive treatment either as a sole therapy or in conjunction with conventional surgery or radiotherapy or chemotherapy. The several classes of chemotherapeutic agents that are listed therein include anti-angiogenic agents such as those which inhibit VEGF such as the compounds disclosed in International Patent Applications WO 97/22596, WO 97/30035, WO 97/32856 and WO 98/13354 and those that work by other mechanisms (for example linomide, inhibitors of integrin αtvβ3 function and angiostatin).

The Present Invention

The present invention relates to ways in which an anti-angiogenic and/or an anti-cancer effect, especially an anti-tumour effect, for example that based in part on the anti-angiogenic effect of a VEGF receptor tyrosine kinase inhibitor, may be produced in a warm-blooded animal such as a human being without causing the hypertension that is associated with the use of an anti-angiogenic agent.

Hypertension is a prevalent cardiovascular disorder that affects many millions of people and, despite the availability of several classes of anti-hypertensive agents, cardiovascular disease remains an important cause of patient morbidity and mortality. Accordingly, it may be useful to counter the sustained increase in blood pressure that occurs when an anti-angiogenic agent such as a VEGP receptor tyrosine kinase inhibitor is administered.

According to the present invention there is provided the use of an anti-angiogenic agent in combination with an inhibitor of the Src family of non-receptor tyrosine kinases (hereinafter a Src kinase inhibitor) in the manufacture of a medicament for use in the substantially normotensive treatment in a warm-blooded mammal such as a human being of a disease state associated with angiogenesis, the Src kinase inhibitor being administered in an amount effective to counteract substantially the hypertension induced by the anti-angiogenic agent.

According to a further feature of the present invention there is provided a method for the substantially normotensive treatment in a warm-blooded mammal such as a human being of a disease state associated with angiogenesis which comprises the administration of an effective amount of an anti-angiogenic agent in combination with a Src kinase inhibitor, said Src kinase inhibitor being administered in an amount effective to counteract substantially the hypertension induced by said anti-angiogenic agent.

It will be appreciated that disease states that have been associated with angiogenesis include cancer, diabetes, psoriasis, rheumatoid arthritis, Kaposi's sarcoma, haemangioma, lymphoedema, acute and chronic nephropathies, atheroma, arterial restenosis, autoimmune diseases, acute inflammation, excessive scar formation and adhesions, endometriosis, dysfunctional uterine bleeding and ocular diseases with retinal vessel proliferation including age-related macular degeneration. Cancer may affect any tissue and includes leukaemia, multiple myeloma and lymphoma. In particular, application of the invention is expected to slow advantageously the growth of primary and recurrent solid tumours of, for example, the colon, breast, prostate, lungs and skin. Further, application of the invention is expected to inhibit any form of cancer associated with VEGF including leukaemia, mulitple myeloma and lymphoma and also, for example, the growth of those primary and recurrent solid tumours which are associated with VEGF, especially those tumours which are significantly dependent on VEGF for their growth and spread, including for example, certain tumours of the colon, breast, prostate, lung, vulva and skin.

It is to be understood that the term “in combination with” that is used in the definition of various aspects of the present invention envisages the simultaneous, separate or sequential administration of the components of the combination. In one aspect of the invention, “in combination with” envisages simultaneous administration of the anti-angiogenic agent and the Src kinase inhibitor. In a further aspect of the invention, “in combination with” envisages sequential administration of those agents. In another aspect of the invention, “in combination with” envisages separate administration of those agents. Where the administration of those agents is sequential or separate, the delay in administering the second component should not be such as to lose the benefit of the counter-balancing effect on blood pressure that is an aim of the combination therapy of the present invention. Thus, for the avoidance of doubt, one aspect of the present invention provides the use of an anti-angiogenic agent in combination with a Src kinase inhibitor in the manufacture of a medicament for administration simultaneously, sequentially or separately for use in the substantially normotensive treatment in a warm-blooded mammal such as a human being of a disease state associated with angiogenesis, the Src kinase inhibitor being administered in an amount effective to counteract substantially the hypertension induced by the anti-angiogenic agent.

The present invention also provides the use of an anti-angiogenic agent in combination with a Src kinase inhibitor in the manufacture of a medicament for use in the treatment in a warm-blooded mammal such as a human being of a disease state associated with angiogenesis characterised in that an appropriate dose of each component of the combination is selected such that the contrasting blood pressure effects associated with the individual use of either component of the combination are substantially counter-balanced.

The present invention also provides a method for the treatment in a warm-blooded mammal such as a human being of a disease state associated with angiogenesis which comprises the administration of an effective amount of an anti-angiogenic agent in combination with an effective amount of a Src kinase inhibitor characterised in that an appropriate dose of each component of the combination is selected such that the contrasting blood pressure effects associated with the individual use of either component of the combination are substantially counter-balanced.

According to a further feature of the present invention there is provided the use of an anti-angiogenic agent in combination with a Src kinase inhibitor in the manufacture of a medicament for use in the substantially normotensive production of an anti-cancer effect in a warm-blooded mammal such as a human being, the Src kinase inhibitor being administered in an amount effective to counteract substantially the hypertension induced by the anti-angiogenic agent.

According to a further feature of the present invention there is provided a method for the substantially normotensive production of an anti-cancer effect in a warm-blooded mammal such as a human being which comprises the administration of an effective amount of an anti-angiogenic agent in combination with a Src kinase inhibitor, said Src kinase inhibitor being administered in an amount effective to counteract substantially the hypertension induced by said anti-angiogenic agent.

Cancers that are amenable to treatment with the combination of the present invention include, in particular, oesophageal cancer, myeloma, hepatocellular, pancreatic and cervical cancer, Ewings tumour, neuroblastoma, Kaposi's sarcoma, ovarian cancer, breast cancer, colorectal cancer, prostate cancer, bladder cancer, melanoma, lung cancer [including non small cell lung cancer (NSCLC) and small cell lung cancer (SCLC)], gastric cancer, head and neck cancer, brain cancer and renal cancer, and haematological cancers such as lymphoma and leukaemia.

In particular, the present invention is useful in the treatment of solid tumours i.e. it provides an anti-tumour effect.

The present invention also provides the use of an anti-angiogenic agent in combination with a Src kinase inhibitor in the manufacture of a medicament for use in the production of an anti-cancer effect in a warm-blooded mammal such as a human being characterised in that an appropriate dose of each component of the combination is selected such that the contrasting blood pressure effects associated with the individual use of either component of the combination are substantially counter-balanced.

The present invention also provides a method for the production of an anti-cancer effect in a warm-blooded mammal such as a human being which comprises the administration of an effective amount of an anti-angiogenic agent in combination with an effective amount of a Src kinase inhibitor characterised in that an appropriate dose of each component of the combination is selected such that the contrasting blood pressure effects associated with the individual use of either component of the combination are substantially counter-balanced.

The anti-cancer treatment of this aspect of the present invention may be assessed by conventional means such as the response rate, the time to disease progression and/or the survival rate. Anti-tumour effects of the present invention include, but are not limited to, inhibition of tumour growth, tumour growth delay, regression of tumour, shrinkage of tumour, increased time to regrowth of tumour on cessation of treatment and slowing of disease progression. For example, it is expected that when the combination of the present invention is administered to a warm-blooded mammal such as a human being who is in need of treatment for solid tumour disease, such a method of treatment will produce an effect on, for example, one or more of the extent of the anti-tumour effect, the response rate, the time to disease progression and the survival rate.

The combination treatment as defined herein requires that an appropriate dose of each component of the combination is selected such that the contrasting blood pressure effects associated with the individual use of either component of the combination are substantially counter-balanced. In one embodiment of the present invention, a first component of the combination is dosed at its conventional dose and the second component is dosed in an amount that substantially counter-balances the blood pressure effect associated with the individual use of the first component. Blood pressure effects are measured by conventional means. Thereby the anti-angiogenic and/or anti-cancer effect is maintained or improved as measured by one or more of the extent of the response, the response rate, the time to disease progression and survival data, in particular the duration of the response. In another embodiment of the present invention, the conventional dose of the first component of the combination may be reduced and the second component is dosed in an amount that substantially counter-balances the blood pressure effect associated with the individual use of the first component and the anti-angiogenic and/or anti-cancer effect is maintained or improved as measured by one or more of the extent of the response, the response rate, the time to disease progression and survival data, in particular the duration of the response. Thereby the anti-angiogenic and/or anti-cancer effect is maintained or improved but with fewer and/or less troublesome side-effects than those that may occur if conventional doses of each component are used.

Anti-angiogenic agents that possess pharmacokinetic properties which provide a reasonable bioavailability when administered chronically lead to an increase in diastolic blood pressure in the rat of about 10 to 30 mm Hg and in human beings of about 10 to 20 mm Hg. Src kinase inhibitors that possess pharmacokinetic properties which provide a reasonable bioavailability after a single dose lead to a decrease in diastolic blood pressure in the rat of about 10 to 25 mm Hg. It will be appreciated that the contrasting blood pressure effects associated with the individual use of either of an anti-angiogenic agent or of a Src kinase inhibitor will be substantially counter-balanced if the Src kinase inhibition reduces the hypertensive effect of the anti-angiogenic agent on diastolic blood pressure to less than about 10 mm Hg, particularly to less than about 5 mm Hg. Further, the blood pressure effects will be substantially counter-balanced if the resultant diastolic blood pressure effect of appropriate doses of a combination of the anti-angiogenic agent and the Src kinase inhibitor is in the range of about −10 to +10 mm Hg, particularly in the range of about −5 to +5 mm Hg. More particularly, the blood pressure effects will be substantially counter-balanced if a substantially normotensive effect is achieved.

Subject to that counter-balancing need, an anti-angiogenic agent as defined herein will generally be administered chronically so that a daily dose in the range, for example, 0.01 mg/kg to 50 mg/kg body weight is received, given if required in divided doses. In general lower doses will be administered when a parenteral route is employed. Thus, for example, for intravenous administration, a daily dose in the range, for example, 0.01 mg/kg to 25 mg/kg body weight will generally be used. Similarly, for administration by inhalation, a daily dose in the range, for example, 0.01 mg/kg to 25 mg/kg body weight will be used. Oral administration is however preferred, particularly in tablet form, to provide a daily dose in the range, for example, 0.01 mg/kg to 10 mg/kg body weight, conveniently 0.01 mg/kg to 5 mg/kg body weight.

Subject to that counter-balancing need, a Src kinase inhibitor as defined herein will generally be administered chronically so that a daily dose in the range, for example, 0.02 mg/kg to 75 mg/kg body weight is received, given if required in divided doses. In general lower doses will be administered when a parenteral route is employed. Thus, for example, for intravenous administration, a daily dose in the range, for example, 0.01 mg/kg to 30 mg/kg body weight will generally be used. Similarly, for administration by inhalation, a daily dose in the range, for example, 0.01 mg/kg to 25 mg/kg body weight will be used. Oral administration is however preferred, particularly in tablet form, to provide a daily dose in the range, for example, 0.02 mg/kg to 15 mg/kg body weight, conveniently 0.02 mg/kg to 5 mg/kg body weight.

According to a further feature of the present invention there is provided the use of an anti-angiogenic agent in combination with a Src kinase inhibitor in the manufacture of a medicament for use in the substantially normotensive production of an improved anti-cancer effect in a warm-blooded mammal such as a human being, the Src kinase inhibitor being administered in an amount effective to counteract substantially the hypertension induced by the anti-angiogenic agent and to improve the anti-cancer activity of the anti-angiogenic agent.

According to a further feature of the present invention there is provided a method for the substantially normotensive production of an improved anti-cancer effect in a warm-blooded mammal such as a human being which comprises the administration of an effective amount of an anti-angiogenic agent in combination with a Src kinase inhibitor, said Src kinase inhibitor being administered in an amount effective to counteract substantially the hypertension induced by said anti-angiogenic agent and to improve the anti-cancer activity of said anti-angiogenic agent.

According to this aspect of the invention, the combination is useful in providing an improved anti-cancer effect, particularly an improved anti-cancer effect comprising both an anti-angiogenic and an anti-invasive effect. According to the present invention, a combination treatment is defined as affording an improved anti-cancer effect if the effect is synergistic. For example, an improved or synergistic anti-cancer effect is one where the effect is therapeutically superior to that achievable on dosing one or other of the components of the combination treatment, as measured by, for example, the extent of the response, the response rate, the time to disease progression or the survival period. For example, the effect of the combination treatment is improved or synergistic if the effect is therapeutically superior to the effect achievable with an anti-angiogenic agent or a Src kinase inhibitor alone. Further, the effect of the combination treatment is improved or synergistic if a beneficial effect is obtained in a group of patients that does not respond (or responds poorly) to an anti-angiogenic agent or a Src kinase inhibitor alone. Further, the effect of the combination treatment is improved or synegistic if a beneficial effect is obtained but with fewer and/or less troublesome side-effects than those that may occur if conventional doses of each component are used.

The present invention also provides the use of an anti-angiogenic agent in combination with a Src kinase inhibitor in the manufacture of a medicament for use in the production of an anti-cancer effect in a warm-blooded mammal such as a human being characterised in that:

(i) an improved anti-cancer effect is obtained; and

(ii) an appropriate dose of each component of the combination is selected such that the contrasting blood pressure effects associated with the individual use of either component of the combination are substantially counter-balanced.

The present invention also provides a method for the production of an anti-cancer effect in a warm-blooded mammal such as a human being which comprises the administration of an effective amount of an anti-angiogenic agent in combination with an effective amount of a Src kinase inhibitor characterised in that :

(i) an improved anti-cancer effect is obtained; and

(ii) an appropriate dose of each component of the combination is selected such that the contrasting blood pressure effects associated with the individual use of either component of the combination are substantially counter-balanced.

According to a further feature of the present invention there is provided the use of an anti-angiogenic agent in combination with a Src kinase inhibitor in the manufacture of a medicament for use in the substantially normotensive production in a warm-blooded mammal such as a human being of an improved anti-cancer effect comprising both an anti-angiogenic and an anti-invasive effect, the Src kinase inhibitor being administered in an amount effective to counteract substantially the hypertension induced by the anti-angiogenic agent and to improve the anti-cancer activity of the anti-angiogenic agent.

According to a further feature of the present invention there is provided a method for the substantially normotensive production in a warm-blooded mammal such as a human being of an improved anti-cancer effect comprising both an anti-angiogenic and an anti-invasive effect which comprises the administration of an effective amount of an anti-angiogenic agent in combination with a Src kinase inhibitor, said Src kinase inhibitor being administered in an amount effective to counteract substantially the hypertension induced by said anti-angiogenic agent and to improve the anti-cancer activity of said anti-angiogenic agent.

The present invention also provides the use of an anti-angiogenic agent in combination with a Src kinase inhibitor in the manufacture of a medicament for use in the production of an anti-cancer effect in a warm-blooded mammal such as a human being characterised in that:

(i) an improved anti-cancer effect is obtained comprising both an anti-angiogenic and an anti-invasive effect; and

(ii) an appropriate dose of each component of the combination is selected such that the contrasting blood pressure effects associated with the individual use of either component of the combination are substantially counter-balanced.

The present invention also provides a method for the production of an anti-cancer effect in a warm-blooded mammal such as a human being which comprises the administration of an effective amount of an anti-angiogenic agent in combination with an effective amount of a Src kinase inhibitor characterised in that:

(i) an improved anti-cancer effect is obtained comprising both an anti-angiogenic and an anti-invasive effect; and

(ii) an appropriate dose of each component of the combination is selected such that the contrasting blood pressure effects associated with the individual use of either component of the combination are substantially counter-balanced.

According to a further feature of the present invention there is provided the use of an anti-angiogenic agent in combination with a Src kinase inhibitor in the manufacture of a medicament for use in the substantially normotensive production in a warm-blooded mammal such as a human being of an improved anti-tumour effect, the Src kinase inhibitor being administered in an amount effective to counteract substantially the hypertension induced by the anti-angiogenic agent and to improve the anti-tumour activity of the anti-angiogenic agent.

According to a further feature of the present invention there is provided a method for the substantially normotensive production of an improved anti-tumour effect in a warm-blooded mammal such as a human being which comprises the administration of an effective amount of an anti-angiogenic agent in combination with a Src kinase inhibitor, said Src kinase inhibitor being administered in an amount effective to counteract substantially the hypertension induced by said anti-angiogenic agent and to improve the anti-tumour activity of said anti-angiogenic agent.

The present invention also provides the use of an anti-angiogenic agent in combination with a Src kinase inhibitor in the manufacture of a medicament for use in the prevention or treatment of solid tumour disease in a warm-blooded mammal such as a human being characterised in that:

(i) an improved anti-tumour effect is obtained; and

(ii) an appropriate dose of each component of the combination is selected such that the contrasting blood pressure effects associated with the individual use of either component of the combination are substantially counter-balanced.

The present invention also provides a method for the prevention or treatment of solid tumour disease in a warm-blooded mammal such as a human being which comprises the administration of an effective amount of an anti-angiogenic agent in combination with an effective amount of a Src kinase inhibitor characterised in that

(i) an improved anti-tumour effect is obtained; and

(ii) an appropriate dose of each component of the combination is selected such that the contrasting blood pressure effects associated with the individual use of either component of the combination are substantially counter-balanced.

According to a further feature of the present invention there is provided the use of an anti-angiogenic agent in combination with a Src kinase inhibitor in the manufacture of a medicament for use in the substantially normotensive production in a warm-blooded mammal such as a human being of an improved anti-tumour effect comprising both an anti-angiogenic and an anti-invasive effect, the Src kinase inhibitor being administered in an amount effective to counteract substantially the hypertension induced by the anti-angiogenic agent and to improve the anti-tumour activity of the anti-angiogenic agent.

According to a further feature of the present invention there is provided a method for the substantially normotensive production in a warm-blooded mammal such as a human being of an improved anti-tumour effect comprising both an anti-angiogenic and an anti-invasive effect which comprises the administration of an effective amount of an anti-angiogenic agent in combination with a Src kinase inhibitor, said Src kinase inhibitor being administered in an amount effective to counteract substantially the hypertension induced by said anti-angiogenic agent and to improve the anti-tumour activity of said anti-angiogenic agent.

The present invention also provides the use of an anti-angiogenic agent in combination with a Src kinase inhibitor in the manufacture of a medicament for use in the prevention or treatment of solid tumour disease in a warm-blooded mammal such as a human being characterised in that:

(i) an improved anti-tumour effect is obtained comprising both an anti-angiogenic and an anti-invasive effect; and

(ii) an appropriate dose of each component of the combination is selected such that the contrasting blood pressure effects associated with the individual use of either component of the combination are substantially counter-balanced.

The present invention also provides a method for the prevention or treatment of solid tumour disease in a warm-blooded mammal such as a human being which comprises the administration of an effective amount of an anti-angiogenic agent in combination with an effective amount of a Src kinase inhibitor characterised in that

(i) an improved anti-tumour effect is obtained comprising both an anti-angiogenic and an anti-invasive effect; and

(ii) an appropriate dose of each component of the combination is selected such that the contrasting blood pressure effects associated with the individual use of either component of the combination are substantially counter-balanced.

According to a further feature of the present invention there is provided the use of an inhibitor of VEGF receptor tyrosine kinases in combination with a Src kinase inhibitor in the manufacture of a medicament for use in the substantially normotensive production in a warm- blooded mammal such as a human being of an improved anti-tumour effect, the Src kinase inhibitor being administered in an amount effective to counteract substantially the hypertension induced by the inhibitor of VEGF receptor tyrosine kinases and to improve the anti-tumour activity of the inhibitor of VEGF receptor tyrosine kinases.

According to a further feature of the present invention there is provided a method for the substantially normotensive production of an improved anti-tumour effect in a warm-blooded mammal such as a human being which comprises the administration of an effective amount of an inhibitor of VEGF receptor tyrosine kinases in combination with a Src kinase inhibitor, said Src kinase inhibitor being administered in an amount effective to counteract substantially the hypertension induced by said inhibitor of VEGP receptor tyrosine kinases and to improve the anti-tumour activity of said inhibitor of VEGF receptor tyrosine kinases.

The present invention also provides the use of an inhibitor of VEGF receptor tyrosine kinases in combination with a Src kinase inhibitor in the manufacture of a medicament for use in the prevention or treatment of solid tumour disease in a warm-blooded mammal such as a human being characterised in that:

(i) an improved anti-tumour effect is obtained; and

(ii) an appropriate dose of each component of the combination is selected such that the contrasting blood pressure effects associated with the individual use of either component of the combination are substantially counter-balanced.

The present invention also provides a method for the prevention or treatment of solid tumour disease in a warm-blooded mammal such as a human being which comprises the administration of an effective amount of an inhibitor of VEGF receptor tyrosine kinases in combination with an effective amount of a Src kinase inhibitor characterised in that

(i) an improved anti-tumour effect is obtained; and

(ii) an appropriate dose of each component of the combination is selected such that the contrasting blood pressure effects associated with the individual use of either component of the combination are substantially counter-balanced.

According to a further feature of the present invention there is provided the use of an inhibitor of VEGF receptor tyrosine kinases in combination with a Src kinase inhibitor in the manufacture of a medicament for use in the substantially normotensive production in a warm-blooded mammal such as a human being of an improved anti-tumour effect comprising both an anti-angiogenic and an anti-invasive effect, the Src kinase inhibitor being administered in an amount effective to counteract substantially the hypertension induced by the inhibitor of VEGF receptor tyrosine kinases and to improve the anti-tumour activity of the inhibitor of VEGF receptor tyrosine kinases.

According to a further feature of the present invention there is provided a method for the substantially normotensive production in a warm-blooded mammal such as a human being of an improved anti-tumour effect comprising both an anti-angiogenic and an anti-invasive effect which comprises the administration of an effective amount of an inhibitor of VEGF receptor tyrosine kinases in combination with a Src kinase inhibitor, said Src kinase inhibitor being administered in an amount effective to counteract substantially the hypertension induced by said inhibitor of VEGF receptor tyrosine kinases and to improve the anti-tumour activity of said inhibitor of VEGF receptor tyrosine kinases.

The present invention also provides the use of an inhibitor of VEGF receptor tyrosine kinases in combination with a Src kinase inhibitor in the manufacture of a medicament for use in the prevention or treatment of solid tumour disease in a warm-blooded mammal such as a human being characterised in that:

(i) an improved anti-tumour effect is obtained comprising both an anti-angiogenic and an anti-invasive effect; and

(ii) an appropriate dose of each component of the combination is selected such that the contrasting blood pressure effects associated with the individual use of either component of the combination are substantially counter-balanced.

The present invention also provides a method for the prevention or treatment of solid tumour disease in a warm-blooded mammal such as a human being which comprises the administration of an effective amount of an inhibitor of VEGF receptor tyrosine kinases in combination with an effective amount of a Src kinase inhibitor characterised in that:

(i) an improved anti-tumour effect is obtained comprising both an anti-angiogenic and an anti-invasive effect; and

(ii) an appropriate dose of each component of the combination is selected such that the contrasting blood pressure effects associated with the individual use of either component of the combination are substantially counter-balanced.

According to a further feature of the present invention there is provided the use of an inhibitor of VEGF receptor tyrosine kinases in combination with a Src kinase inhibitor in the manufacture of a medicament for use in a warm-blooded mammal such as a human being in the substantially normotensive prevention or treatment of those tumours which are sensitive to inhibition of one or both of VEGF receptor tyrosine kinase and Src kinase, the Src kinase inhibitor being administered in an amount effective to counteract substantially the hypertension induced by the inhibitor of VEGF receptor tyrosine kinases.

According to a further feature of the present invention there is provided a method for the substantially normotensive prevention or treatment of those tumours which are sensitive to inhibition of one or both of VEGF receptor tyrosine kinase and Src kinase which comprises the administration to a warm-blooded mammal such as a human being of an effective amount of an inhibitor of VEGF receptor tyrosine kinases in combination with a Src kinase inhibitor, said Src kinase inhibitor being administered in an amount effective to counteract substantially the hypertension induced by said inhibitor of VEGF receptor tyrosine kinases.

The present invention also provides the use of an inhibitor of VEGF receptor tyrosine kinases in a combination with a Src kinase inhibitor in the manufacture of a medicament for use in a warm-blooded mammal such as a human being in the prevention or treatment of those tumours which are sensitive to inhibition of one or both of VEGF receptor tyrosine kinase and Src kinase characterised in that an appropriate dose of each component of the combination is selected such that the contrasting blood pressure effects associated with the individual use of either component of the combination are substantially counter-balanced.

The present invention also provides a method for the prevention or treatment of those tumours which are sensitive to inhibition of one or both of VEGF receptor tyrosine kinase and Src kinase which comprises the administration to a warm-blooded mammal such as a human being of an effective amount of an inhibitor of VEGF receptor tyrosine kinases in combination with an effective amount of a Src kinase inhibitor characterised in that an appropriate dose of each component of the combination is selected such that the contrasting blood pressure effects associated with the individual use of either component of the combination are substantially counter-balanced.

A suitable anti-angiogenic agent for use in any aspect of the present invention is any agent which inhibits the growth and maintenance of new blood vessels by inhibiting VEGF signalling. Suitable anti-angiogenic agents include:

(i) inhibitors of one or more VEGF receptor tyrosine kinases;

(ii) VEGF antibodies such as bevacizumab (Avastin™, Genentech) and VEGF receptor antibodies such as IMC-1C11 (ImClone Systems); and

(iii) inhibitors of VEGF expression such as RPI 4610 (Angiozyme™, Chiron Corporation/Ribozyme Pharmaceuticals).

A suitable anti-angiogenic agent is also any agent which inhibits the growth and maintenance of new blood vessels by vascular targeting. Suitable vascular targeting agents include Combretastatin A4 phosphate (Oxigene, Bristol Myers Squibb, U.S. Pat. No. 4,996,237); AVE-8062; Exherin™ (Adherex); 5,6-dimethylxanthenone-4-acetic acid (DMAA); and the vascular damaging agents described in International Patent Applications WO 99/02166 and WO 00/40529. A preferred vascular damaging agent is N-acetylcolchinol-O-phosphate Example 1 of International Patent Application WO 99/02166) which is also known as ZD6126 (AstraZeneca).

Conveniently, the anti-angiogenic agent is an inhibitor of one or more VEGF receptor tyrosine kinases. Such compounds include ZD6474 (AstraZeneca, Example 2 of International Patent Application WO 01/32651), vatalanib™ (PTK787/ZK 222584; Novartis/Schering, International Patent Application WO 98/35958), SU11248 (Pharmacia, International Patent Application WO 01/60814), CP-547632 (Pfizer, International Patent Application WO 99/62890) and CEP-7055 (Cephalon).

A suitable anti-angiogenic agent is an inhibitor of the VEGF receptor tyrosine kinase enzymes that, in general, possesses one or more of:

(i) IC₅₀ values against Flt-1 and/or KDR in the range, for example, 0.001 to 5μM, preferably in the range, for example, 0.001 to 0.5 μM;

(ii) greater inhibitory potency against VEGF receptor kinases than against Src kinase; and

(iii) pharmacokinetic properties which provide a reasonable bioavailability when administered to a warm-blooded animal, especially when administered chronically.

The activity of a compound against VEGF receptor tyrosine kinases such as Flt-1 and KDR may be assessed using appropriate conventional assays such as those described in, for example, International Patent Application WO 98/13354.

Compounds which are inhibitors of VEGF receptor tyrosine kinases are described in, for example, International Patent Applications WO 97/22596, WO 97/30035, WO 97/32856, WO 97/34876, WO 97/42187, WO 98/13354, WO 98/13350, WO 99/10349, WO 00/21955, WO 00/47212, WO 01/32651, WO 01/66099, WO 01/77085, WO 02/12226, WO 02/12227, WO 02/12228 and WO 02/16348 and in WO 03/064413 (arising from co-pending International Patent Application No. PCT/GB03/00343).

Selective inhibitors of the VEGF receptor tyrosine kinase enzymes possess greater inhibitory potency against VEGF receptor kinases than against other tyrosine kinase enzymes. Suitable selective VEGF receptor tyrosine kinase inhibitors for use in the present invention possess potent inhibitory activity against VEGF receptor tyrosine kinases such as Flt-1 and KDR that have been shown to bind VEGF with high affinity whilst possessing less potent inhibitory activity against other tyrosine kinase enzymes such as other receptor tyrosine kinases or against non-receptor tyrosine kinases, in particular against the Src family of non-receptor tyrosine kinases, for example c-Src and/or c-Yes. Given the above-mentioned anti-hypertensive effect of Src kinase inhibition, it will be appreciated that compounds exhibiting such VEGP receptor selectivity provide a greater degree of hypertension than those which possess significant Src kinase inhibitory activity.

In general, a VEGF receptor tyrosine kinase inhibitor for use in the present invention possesses a KDR IC₅₀ in the range, for example, 0.001-1 μM and a Src kinase IC₅₀ in the range, for example, 0.01-100 μM. The selectivity of the VEGF receptor tyrosine kinase inhibition of a compound may be assessed by dividing the Src kinase IC₅₀ by the KDR IC₅₀ to provide a ratio. A compound possesses substantially better potency against VEGF receptor tyrosine kinases than against Src kinase when the ratio of Src kinase IC₅₀ to KDR IC₅₀ is:

(i) in general, in the range, for example, of about 2 to 1,000;

(ii) particularly, in the range, for example, of about 10 to 1,000; and

(iii) preferably, in the range, for example, of about 50 to 1,000.

Suitable compounds which possess such selective VEGF receptor tyrosine kinase inhibitory properties are described in, for example, International Patent Applications WO 97/22596, WO 97/30035, WO 98/13354, WO 00/47212, WO 01/32651 and WO 01/77085, and in WO 03/064413 (arising from co-pending International Patent Application No. PCT/GB03/00343).

Particular selective VEGF receptor tyrosine kinase inhibitors are described in, for example, International Patent Applications WO 00/47212 and WO 01/32651 and in WO 03/064413 (arising from co-pending International Patent Application No. PCT/GB03/00343).

A suitable Src kinase inhibitor for use in any aspect of the present invention is a compound that possesses inhibitory activity against one or more of the Src family of non-receptor tyrosine kinases, for example a suitable Src kinase inhibitor possesses one or more of:

(i) an IC₅₀ value against Src kinase in the range, for example, 0.001 to 5 μM, preferably in the range, for example, 0.001 to 0.5 μM;

(ii) greater inhibitory potency against Src kinase than against VEGF receptor kinases; and

(iii) pharmacokinetic properties which provide a reasonable bioavailability when administered to a warm-blooded animal, especially when administered chronically.

The potency of a compound as a Src kinase inhibitor may be assessed using a conventional Elisa assay such as that described in, for example, International Patent Application WO 01/94341.

Compounds which possess Src kinase inhibitory properties are described in, for example, International Patent Applications WO 01/94341, WO 02/16352, WO 02/30924, WO 02/30926, WO 02/34744, WO 02/085895, WO 02/092577, WO 02/092578, WO 02/092579 and WO 03/008409 and in co-pending International Application PCT/GB03/04703 (arising from European Patent Application No. 02292736.2).

It is disclosed in Journal Medicinal Chemistry, 2001, 44, 822-833 and 3965-3977 that certain 4-anilino-3-cyanoquinoline derivatives are useful for the inhibition of Src-dependent cell proliferation. The 4-anilino-3-cyanoquinoline Src inhibitor known as SKI 606 is described in Cancer Research, 2003, 63, 375.

Other compounds which possess Src kinase inhibitory properties are described in, for example, International Patent Applications WO 96/10028, WO 97/07131, WO 97/08193, WO 97/16452, WO 97/28161, WO 97/32879 and WO 97/49706.

Other compounds which possess Src kinase inhibitory properties are described in, for example, International Patent Application WO 03/013540 [particularly the compounds disclosed therein by way of Formulae I to VIII and compounds based on Formulae VII and VIII but wherein the 2,6-dimethylphenyl group is replaced by a 2,6-dichlorophenyl or a 2-chloro-6-methylphenyl group.

Other compounds which possess Src kinase inhibitory properties are described in, for example, J Bone Mineral Research, 1999, 14 (Suppl. 1), S487, Molecular Cell, 1999, 3, 639-647, Journal Medicinal Chemistry, 1997, 40, 2296-2303, Journal Medicinal Chemistry, 1998, 41, 3276-3292 and Bioorganic & Medicinal Chemistr Letters, 2002, 12, 1361 and 3153.

Particular Src kinase inhibitors include:
(i) 4-amino-5-(3-methoxyphenyl)-7-{4-[2-(2-methoxyethylamino)ethoxy]phenyl}-pyrrolo[2,3-d]pyrimidine and 4-amino-5-(3-methoxyphenyl)-7-(4-{2-[di-(2-methoxyethyl)amino]ethoxy}phenyl)pyrrolo[2,3-d]pyrimidine which are obtainable by methods described in International Patent Application WO 96/10028;
(ii) 4-amino-7-tert-butyl-5-(4-tolyl)pyrazolo[3,4-d]pyrimidine which is also known as PP1 and is described in Molecular Cell, 1999, 3, 639-648;
(iii) 2-(2,6-dichloroanilino)-6,7-dimethyl-1,8-dihydroimidazo[4,5-h]isoquinolin-9-one and 2-(2,6-dichloroanilino)-7-[(E)-3-diethylaminoprop-1-enyl]-6-methyl-1,8-dihydroimidazo[4,5-h]isoquinolin-9-one which are obtainable by methods described in Journal Medicinal Chemistry, 2002, 45, 3394;
(iv) 1-[6-(2,6-dichlorophenyl)-2-(4-diethylaminobutyl)pyrido[2,3-d]pryidin-7-yl]-3-ethylurea which is obtainable by methods described in Journal Medicinal Chemistry, 1997, 30 40, 2296-2303 and Journal Medicinal Chemistry, 2001, 44, 1915;
(v) 6-(2,6-dichlorophenyl)-2-[4-(2-diethylaminoethoxy)anilino]-8-methyl-8H-pyrido[2,3-d]pyrimidin-7-one which is also known as PD166285 and is described in J. Pharmacol. Exp. Ther., 1997, 283, 1433-1444;
(vi) the compound known as PD162531 which is described in Mol. Biol. Cell, 2000, 11, 51-64;
(vii) the compound known as PD166326 which is described in Biochem. Pharmacol., 2000, 60, 885-898; and
(viii) the compound known as PD173955 which is described in Cancer Research, 1999, 59, 6145-6152.

Other compounds which may possess Src kinase inhibitory properties are described in, for example, International Patent Applications WO 02/079192, WO 03/000188, WO 03/000266, WO 03/000705, WO 02/083668, WO 02/092573, WO 03/004492, WO 00/49018, WO 03/013541, WO 01/00207, WO 01/00213 and WO 01/00214.

Selective Src kinase inhibitors possess greater inhibitory potency against Src kinase than against VEGF receptor kinases. Suitable selective Src kinase inhibitors for use in the present invention possess potent inhibitory activity against the Src family of non-receptor tyrosine kinases, for example by inhibition of c-Src and/or c-Yes, whilst possessing less potent inhibitory activity against other tyrosine kinase enzymes such as the receptor tyrosine kinases, in particular against VEGF receptor tyrosine kinases such as Flt-1 and KDR that have been shown to bind VEGF with high affinity. Compounds exhibiting such Src selectivity provide a greater degree of hypotension than those which possess significant VEGF receptor tyrosine kinase inhibitory activity.

In general, a Src kinase inhibitor for use in the present invention possesses a Src kinase IC₅₀ in the range, for example, 0.001-1 μM and a KDR IC₅₀ in the range, for example, 0.1-100 μM. The selectivity of the Src kinase activity of a compound may be assessed by dividing the KDR IC₅₀ by the Src kinase IC₅₀ to provide a ratio. When it is stated that the Src kinase inhibitor possesses substantially better potency against Src kinase than against VEGF receptor tyrosine kinases, this means that the ratio of KDR IC₅₀ to Src kinase IC₅₀ is:
(i) in general, in the range, for example, of about 5 to 10,000;
(ii) particularly, in the range, for example, of about 25 to 10,000; and
(iii) preferably, in the range, for example, of about 100 to 10,000.

Suitable compounds which possess such selective Src kinase inhibitory properties are described in, for example, International Patent Applications WO 01/94341, WO 02/16352, WO 02/30924, WO 02/30926, WO 02/34744, WO 02/085895, WO 02/092577, WO 02/092578, WO 02/092579 and WO 03/008409 and in co-pending International Application PCT/GB03/04703 (arising from European Patent Application No. 02292736.2).

Particular selective Src kinase inhibitors are described in, for example, International Patent Applications WO 01/94341, WO 02/16352, WO 02/085895, WO 02/092577, WO 02/092578 and WO 02/092579 and in co-pending International Application PCT/GB03/04703 (arising from European Patent Application No. 02292736.2).

Further particular inhibitors of VEGF receptor tyrosine kinases and Src kinase inhibitors that may be used in the present invention include those compounds that possess appropriate pharmacokinetic properties after administration to a warm-blooded mammal such as a rat, dog or human being, particularly after oral administration. Such compounds provide suitable blood levels and a reasonable bioavailability when administered acutely, particularly when administered chronically. In general, the VEGF receptor tyrosine kinase inhibitor and the Src kinase inhibitor as defined hereinbefore will be administered chronically over a number of days to allow assessment of the anti-angiogenic and/or anti-cancer effect of the combination, particularly of the effect on solid tumour disease, and of any effect on the patient's blood pressure. In general, oral administration is preferred, particularly using tablet forms.

In general, each of an inhibitor of VEGF receptor tyrosine kinases and a Src kinase inhibitor that possesses suitable pharmacokinetic properties when administered to a warm-blooded mammal such as a human being possesses one or more of the following pharmacokinetic parameters:
(i) Compound Clearance of less than about 75% of hepatic blood flow (hepatic blood flow in the human is about 25 ml/min/kg, in the dog is about 35 ml/min/kg and in the rat is about 75 ml/min/kg);

(ii) a Volume of Distribution of less than about 30 L/kg;

(iii) a bioavailability of more than about 20%; and

(iv) an elimination half life in the range, for example, of about 0.2 to 15 hours.

In general, each of a particular VEGF receptor tyrosine kinase inhibitor and a particular Src kinase inhibitor that possesses suitable pharmacokinetic properties when administered to a warm-blooded mammal such as a human being possesses one or more of the following pharmacokinetic parameters:

(i) Compound Clearance of less than about 50% of hepatic blood flow;

(ii) a Volume of Distribution of less than about 20 L/kg;

(iii) a bioavailability of more than about 30%; and

(iv) an elimination half life in the range, for example, of about 0.5 to 10 hours.

In general, each of a more particular VEGF receptor tyrosine kinase inhibitor and a more particular Src kinase inhibitor that possesses suitable pharmacokinetic properties when administered to a warm-blooded mammal such as a human being possesses one or more of the following pharmacokinetic parameters:

(i) Compound Clearance of less than about 40% of hepatic blood flow;

(ii) a Volume of Distribution of less than about 10 L/kg;

(iii) a bioavailability of more than about 40%; and

(iv) an elimination half life in the range, for example, of about 1 to 7.5 hours.

Particular selective VEGF receptor tyrosine kinase inhibitors that may be used for chronic administration in the present invention are described in, for example, International Patent Applications WO 00/47212 and WO 01/32651 and in WO 03/064413 (arising from co-pending International Patent Application No. PCT/GB03/00343).

Particular VEGF receptor tyrosine kinase inhibitors include the following compounds from International Patent Application No. WO 00/47212:
6-methoxy-4-(2-methylindol-5-yloxy)-7-(3-(pyrrolidin-1-yl)propoxy)quinazoline,
4-(4-fluoroindol-5-yloxy)-6-methoxy-7-(1 -methylpiperidin-4-ylmethoxy)quinazoline,
4-(4-fluoroindol-5-yloxy)-6-methoxy-7-(3-(4-methylpiperazin-1-yl)propoxy)quinazoline,
4-(6-fluoroindol-5-yloxy)-6-methoxy-7-(3-(pyrrolidin-1-yl)propoxy)quinazoline,
4-(4-fluoroindol-5-yloxy)-6-methoxy-7-(3-(pyrrolidin-1-yl)propoxy)quinazoline,
4-(4-fluoroindol-5-yloxy)-6-methoxy-7-(3-piperidinopropoxy)quinazoline,
4-(4-fluoro-2-methylindol-5-yloxy)-6-methoxy-7-(3-(pyrrolidin-1-yl)propoxy)quinazoline,
4-(4-fluoro-2-methylindol-5-yloxy)-6-methoxy-7-(3-piperidinopropoxy)quinazoline,
4-(4-fluoro-2-methylindol-5-yloxy)-6-methoxy-7-((1-methylpiperidin-4-yl)methoxy)quinazoline,
4-(4-fluoro-2-methylindol-5-yloxy)-6-methoxy-7-(3-(4-methylpiperazin-1-yl)propoxy)quinazoline,
4-(4-fluoroindol-5-yloxy)-6-methoxy-7-(2-(1-methylpiperidin-4-yl)ethoxy)quinazoline,
(2R)-7-(2-hydroxy-3-(pyrrolidin-1-yl)propoxy)-4-(4-fluoro-2-methylindol-5-yloxy)-6-methoxyquinazoline, and
4-(4-fluoro-2-methylindol-5-yloxy)-6-methoxy-7-(2-(1-methylpiperidin-4-yl)ethoxy)quinazoline;
and pharmaceutically-acceptable salts thereof.

Further particular VEGF receptor tyrosine kinase inhibitors include the following compounds from International Patent Application No. WO 01/3265:
4-(4-chloro-2-fluoroanilino)-6-methoxy-7-(1-methylpiperidin-4-ylmethoxy)quinazoline,
4-(2-fluoro-4-methylanilino)-6-methoxy-7-(1-methylpiperidin-4-ylmethoxy)quinazoline,
4-(4-bromo-2-fluoroanilino)-6-methoxy-7-(1-methylpiperidin-4-ylmethoxy)quinazoline,
4-(4-chloro-2,6-difluoroanilino)-6-methoxy-7-(1-methylpiperidin-4-ylmethoxy)quinazoline,
4-(4-bromo-2,6-difluoroanilino)-6-methoxy-7-(1-methylpiperidin-4-ylmethoxy)quinazoline,
4-(4-chloro-2-fluoroanilino)-6-methoxy-7-(piperidin-4-ylmethoxy)quinazoline,
4-(2-fluoro-4-methylanilino)-6-methoxy-7-(piperidin-4-ylmethoxy)quinazoline,
4-(4-bromo-2-fluoroanilino)-6-methoxy-7-(piperidin-4-ylmethoxy)quinazoline,
4-(4-chloro-2,6-difluoroanilino)-6-methoxy-7-(piperidin-4-ylmethoxy)quinazoline, and
4-(4-bromo-2,6-difluoroanilino)-6-methoxy-7-(piperidin-4-ylmethoxy)quinazoline;
and pharmaceutically-acceptable salts thereof.

Further particular VEGP receptor tyrosine kinase inhibitors include the following compounds from WO 03/064413 (arising from co-pending International Patent Application No. PCI/GB03/00343):
6-(3-(4-acetylpiperazin-1-yl)propoxy)-4-(4-fluoro-2-methylindol-5-yloxy)-7-methoxyquinazoline,
7-(3-(4-acetylpiperazin-1-yl)propoxy)-4-(7-azaindol-5-yloxy)-6-methoxyquinazoline,
4-(7-azaindol-5-yloxy)-6-methoxy-7-(3-(4-methylsulphonylpiperazin-1-yl)propoxy)quinazoline,
4-(7-azaindol-5-yloxy)-6-methoxy-7-[2-(N-Methyl-N-prop-2-yn-1-ylamino)ethoxy]quinazoline,
4-(4-fluoro-2-methylindol-5-yloxy)-7-methoxy-6-(3-(4-methylsulphonylpiperazin-1-yl)propoxy)quinazoline,
4-(4-fluoro-2-methylindol-5-yloxy)-6-methoxy-7-(3-(4-methylsulphonylpiperazin-1-yl)propoxy)quinazoline,
6-(3-(4-acetylpiperazin-1-yl)propoxy)-4-(4-fluoroindol-5-yloxy)-7-methoxyquinazoline,
7-[(1-acetylpiperidin-4-yl)methoxy]-4-[(4-fluoro-2-methyl-1H-indol-5-yl)oxy]-6-methoxyquinazoline,
7-[(2S)-1-acetylpyrrolidin-2-ylmethioxy]-4-[(4-fluoro-2-methyl-1H-indol-5-yl)oxy]-6-methoxyquinazoline,
7-[(2R)-1-acetylpyrrolidin-2-ylmethoxy]-4-[(4-fluoro-2-methyl-1H-indol-5-yl)oxy]-6-methoxyquinazoline,
4-[(4-fluoro-2-methyl-1H-indol-5-yl)oxy]-6-methoxy-7-[1-(2,2,2-trifluoroethyl)piperidin-4-ylmethoxy]quinazoline,
4-[(4-fluoro-2-methyl-1H-indol-5-yl)oxy]-6-methoxy-7-{3-[4-(2,2,2-trifluoroethyl)piperazin-1-yl]propoxy}quinazoline,
4-[(4-fluoro-2-methyl-1H-indol-5-yl)oxy]-6-methoxy-7-{3-[4-(2,2,2-trifluorethyl)piperazin-1-yl]ethoxy}quinazoline,
7-{2-[4-(2-fluoroethyl)piperazin-1-yl]ethoxy}-4-[(4-fluoro-2-methyl-1H-indol-5-yl)oxy]-6-methoxyquinazoline,
7-{2-[2-(4-acetylpiperazin-1-yl)ethoxy]ethoxy}-4-[(4-fluoro-2-methyl-1H-indol-5-yl)oxy]-6-methoxyquinazoline,
4-[(4-fluoro-2-methyl-1H-indol-5-yl)oxy]-7-[(1-isobutyrylpipezidin-4-yl)methoxy]-6-methoxyquinazoline,
4-[(4-fluoro-2-methyl-1H-indol-5-yl)oxy]-7-{[(2R)-1-isobutyrylpyrrolidin-2-yl]methoxy}-6-methoxyquinazoline,
4-[(4-fluoro-2-methyl-1H-indol-5-yl)oxy]-6-methoxy-7-{[1-(methylsulfonyl)piperidin-4-yl]methoxy}quinazoline,
4-[(4-fluoro-2-methyl-1H-indol-5-yl)oxy]-6-methoxy-7-{[(2S)-1-(methylsulfonyl)pyrrolidin-2-yl]methoxy}quinazoline,
4-[(4-fluoro-2-methyl-1H-indol-5-yl)oxy]-6-methoxy-7-[{(2R)-1-(methylsulfonyl)pyrrolidin-2-yl]methoxy}quinazoline,
7-[3-(4-allylpiperazin-1-yl)propoxy]-4-(7-azaindol-5-yloxy)-6-methoxyquinazoline,
4-[(4-fluoro-2-methylindol-5-yl)oxy]-6-methoxy-7-{3-[4-(2-propynyl)piperazin-1-yl]propoxy}quinazoline,
7-{3-[4-(2-fluoroethyl)piperazin-1-yl]propoxy}-4-[(4-fluoro-2-methyl-1H-indol-5-yl)oxy]-6-methoxyquinazoline,
7-[3-(4-acetylpiperazin-1-yl)propoxy]-4-(1H-indol-5-yloxy)-6-methoxyquinazoline,
7-[(2S)-1-carbamoylpyrrolidin-2-ylmethoxy]-4-[(4-fluoro-2-methyl-1H-indol-5-yl)oxy]-6-methoxyquinazoline,
7-{3-[4-carbamoylpiperazin-1-yl]propoxy}-4-[(4-fluoro-2-methyl-1H-indol-5-yl)oxy]-6-methoxyquinazoline,
7-{3-[2,5-dioxo-4-(1-hydroxy-1-methylethyl)imidazolidin-1-yl]propoxy}-4-[(4-fluoro-2-methyl-1H-indol-5-yloxy]-6-methoxyquinazoline,
6-[(1-acetylpiperidin-4-yl)oxy]-4-[(4-fluoro-1H-indol-5-yl)oxy]-7-methoxyquinazoline,
4-[(4-fluoro-1H-indol-5-yl)oxy]-7-methoxy-6-{[1-(methylsulphonyl)piperidin-4-yl]oxy}quinazoline,
4-[(4-fluoro-2-methyl-1H-indol-5-yl)oxy]-6-methoxy-7-{2-[N-methyl-N-(2-propynyl)amino]ethoxy}quinazoline,
7-[3-(4-acetylpiperazin-1-yl)propoxy]-6-methoxy-4-[(2-methyl-1H-indol-5-yl)oxy]quinazoline,
7-[3-(4-acetylpiperazin-1-yl)propoxy]-4-[(4-fluoro-1H-indol-5-yl)oxy]-6-methoxyquinazoline,
7-[3-(4-carbamoylmethylpiperazin-1-yl)propoxy]-4-[(4-fluoro-2-methyl-1H-indol-5-yl)oxy]-6-methoxyquinazoline,
7-{3-[4-(2-fluoroethyl)piperazin-1-yl]propoxy}-6-methoxy-4-[(2-methyl-1H-indol-5-yl)oxy]quinazoline,
4-[(4-fluoro-2-methyl-1H-indol-5-yl)oxy]-7-{(2R)-2-hydroxy-3-[4-prop-2-yn-1-ylpiperazin-1-yl]propoxy}-6-methoxyquinazoline,
7-{(2R)-3-[(1,4-dioxa-8-azaspiro[4.5]dec-8-yl)]-2-hydroxypropoxy}-4-[(4-fluoro-2-methyl-1H-indol-5-yl)oxy]-6-methoxyquinazoline,
7-{(2R)-3-[4-acetylpiperazin-1-yl]-2-hydroxypropoxy}4-[(4-fluoro-2-methyl-1H-indol-5-yl)oxy]-6-methoxyquinazoline,
7-(3-(4-acetylpiperazin-1-yl)propoxy)-4-(4-fluoro-2-methylindol-5-yloxy)-6-methoxyquinazoline, and
7-[2-(4-acetylpiperazin-1-yl)ethoxy]-4-[(4-fluoro-2-methyl-1H-indol-5-yl)oxy]-6-methoxyquinazoline,
and pharmaceutically-acceptable salts thereof.

More particular selective VEGF receptor tyrosine kinase inhibitors include:
4-(4-fluoroindol-5-yloxy)-6-methoxy-7-(1-methylpiperidin-4-ylmethoxy)quinazoline,
4-(4-fluoroindol-5-yloxy)-6-methoxy-7-(3-(4-methylpiperazin-1-yl)propoxy)quinazoline,
4-(4-fluoroindol-5-yloxy)-6-methoxy-7-(3-(pyrrolidin-1-yl)propoxy)quinazoline,
4-(4-fluoroindol-5-yloxy)-6-methoxy-7-(3-piperidinopropoxy)quinazoline,
4-(4-fluoro-2-methylindol-5-yloxy)-6-methoxy-7-(3-(pyrrolidin-1-yl)propoxy)quinazoline,
4-(4-fluoro-2-methylindol-5-yloxy)-6-methoxy-7-(3-piperidinopropoxy)quinazoline,
4-(4-fluoro-2-methylindol-5-yloxy)-6-methoxy-7-((1-methylpiperidin-4-yl)methoxy)quinazoline,
4-(4-fluoro-2-methylindol-5-yloxy)-6-methoxy-7-(3-(4-methylpiperazin-1-yl)propoxy)quinazoline,
4-(4-fluoroindol-5-yloxy)-6-methoxy-7-(2-(1-methylpiperidin-4-yl)ethoxy)quinazoline,
4-(4-fluoro-2-methylindol-5-yloxy)-6-methoxy-7-(2-(1-methylpiperidin-4-yl)ethoxy)quinazoline,
4-(4-chloro-2-fluoroanilino)-6-methoxy-7-(1-methylpiperidin-4-ylmethoxy)quinazoline,
4-(2-fluoro-4-methylanilino)-6-methoxy-7-(1-methylpiperidin-4-ylmethoxy)quinazoline,
4-(4-bromo-2-fluoroanilino)-6-methoxy-7-(N-methylpiperidin-4-ylmethoxy)quinazoline,
4-(4-chloro-2-fluoroanilino)-6-methoxy-7-(piperidin-4-ylmethoxy)quinazoline,
4-(2-fluoro-4-methylanilino)-6-methoxy-7-(piperidin-4-ylmethoxy)quinazoline,
4-(4-bromo-2-fluoroanilino)-6-methoxy-7-(piperidin-4-ylmethoxy)quinazoline,
4-(4-fluoro-2-methylindol-5-yloxy)-6-methoxy-7-(3-(4-methylsulphonylpiperazin-1-yl)propoxy)quinazoline,
4-[(4-fluoro-2-methyl-1H-indol-5-yl)oxy]-6-methoxy-7-{3-[4-(2,2,2-trifluoroethyl)piperazin-1-yl]propoxy}quinazoline,
7-{2-[4-(2-fluoroethyl)piperazin-1-yl]ethoxy}-4-[(4-fluoro-2-methyl-1H-indol-5-yl)oxy]-6-methoxyquinazoline,
4-[(4-fluoro-2-methylindol-5-yl)oxy]-6-methoxy-7-{3-[4-(2-propynyl)piperazin-1-yl]propoxy}quinazoline,
7-{3-[4-(2-fluoroethyl)piperazin-1-yl]propoxy}-4-[(4-fluoro-2-methyl-1H-indol-5-yl)oxy]-6-methoxyquinazoline,
7-[3-(4-acetylpiperazin-1-yl)propoxy]-4-[(4-fluoro-1H-indol-5-yl)oxy]-6-methoxyquinazoline,
7-(3-(4-acetylpiperazin-1-yl)propoxy)-4-(4-fluoro-2-methylindol-5-yloxy)-6-methoxyquinazoline, and
7-[2-(4-acetylpiperazin-1-yl)ethoxy]-4-[(4-fluoro-2-methyl-1H-indol-5-yl)oxy]-6-methoxyquinazoline,
or pharmaceutically-acceptable acid-addition salts thereof.

Preferred selective VEGF receptor tyrosine kinase inhibitors include:
4-(4-fluoro-2-methylindol-5-yloxy)-6-methoxy-7-(3-(pyrrolidin-1-yl)propoxy}quinazoline,
4-(4-fluoro-2-methylindol-5-yloxy)-6-methoxy-7-(3-piperidinopropoxy)quinazoline,
4-(4-fluoro-2-methylindol-5-yloxy)-6-methoxy-7-((1-methylpiperidin-4-yl)methoxy)quinazoline,
4-(4-fluoro-2-methylindol-5-yloxy)-6-methoxy-7-(3-(4-methylpiperazin-1-yl)propoxy)quinazoline,
4-(4-fluoro-2-methylindol-5-yloxy)-6-methoxy-7-(2-(1-methylpiperidin-4-yl)ethoxy)quinazoline,
4-(4-chloro-2-fluoroanilino)-6-methoxy-7-(1-methylpiperidin-4-ylmethoxy)quinazoline,
4-(4-bromo-2-fluoroanilino)-6-methoxy-7-(1-methylpiperidin-4-ylmethoxy)quinazoline,
4-(4-chloro-2-fluoroanilino)-6-methoxy-7-(piperidin-4-ylmethoxy)quinazoline,
4-(4-bromo-2-fluoroanilino)-6-methoxy-7-(piperidin-4-ylmethoxy)quinazoline,
4-[(4-fluoro-2-methyl-1H-indol-5-yl)oxy]-6-methoxy-7-{3-[4-(2,2,2-trifluoroethyl)piperazin-1-yl]propoxy)quinazoline,
7-{2-[4-(2-fluoroethyl)piperazin-1-yl]ethoxy}-4-[(4-fluoro-2-methyl-1H-indol-5-yl)oxy]-6-methoxyquinazoline,
4-[(4-fluoro-2-methylindol-5-yl)oxy]-6-methoxy-7-{3-[4-(2-propynyl)piperazin-1-yl]propoxy}quinazoline,
7-{3-[4-(2-fluoroethyl)piperazin-1-yl]propoxy}-4-f(4-fluoro-2-methyl-1H-indol-6-methoxyquinazoline,
7-(3-(4-acetylpiperazin-1-yl)propoxy)-4-(4-fluoro-2-methylindol-5-yloxy)-6-methoxyquinazoline, and
7-[2-(4-acetylpiperazin-1-yl)ethoxy]-4-[(4-fluoro-2-methyl-1H-indol-5-yl)oxy]-6-methoxyquinazoline;
or a pharmaceutically-acceptable acid-addition salt thereof.

A particular preferred VEGF receptor tyrosine kinase inhibitor for use in the invention is 4-(4-fluoro-2-methylindol-5-yloxy)-6-methoxy-7-(3-(pyrrolidin-1-yl)propoxy)quinazoline, or a pharmaceutically-acceptable acid-addition salt thereof.

A further particular preferred VEGF receptor tyrosine kinase inhibitor for use in the invention is 4-(4-fluoro-2-methylindol-5-yloxy)-6-methoxy-7-(3-piperidinopropoxy)quinazoline, or a pharmaceutically-acceptable acid-addition salt thereof.

A further particular preferred VEGF receptor tyrosine kinase inhibitor for use in the invention is 4-(4-fluoro-2-methylindol-5-yloxy)-6-methoxy-7-(3-(4-methylpiperazin-1-yl)propoxy)quinazoline, or a pharmaceutically-acceptable acid-addition salt thereof.

A further particular preferred VEGF receptor tyrosine kinase inhibitor for use in the invention is 4-(4-fluoro-2-methylindol-5-yloxy)-6-methoxy-7-(2-(1-methylpiperidin-4-yl)ethoxy)quinazoline, or a pharmaceutically-acceptable acid-addition salt thereof.

A further particular preferred VEGF receptor tyrosine kinase inhibitor for use in the invention is 4-(4-chloro-2-fluoroanilino)-6-methoxy-7-(1-methylpiperidin-4-ylmethoxy)quinazoline, or a pharmaceutically-acceptable acid-addition salt thereof.

A further particular preferred VEGF receptor tyrosine kinase inhibitor for use in the invention is 4-(4-bromo-2-fluoroanilino)-6-methoxy-7-(1-methylpiperidin-4-ylmethoxy)quinazoline, or a pharmaceutically-acceptable acid-addition salt thereof.

A further particular preferred VEGF receptor tyrosine kinase inhibitor for use in the invention is 4-(4-bromo-2-fluoroanilino)-6-methoxy-7-(piperidin-4-ylmethoxy)quinazoline, or a pharmaceutically-acceptable acid-addition salt thereof.

A further particular preferred VEGF receptor tyrosine kinase inhibitor for use in the invention is 4-[(4-fluoro-2-methylindol-5-yl)oxy]-6-methoxy-7-{3-[4-(2-propynyl)piperazin-1-yl]propoxy}quinazoline, or a pharmaceutically-acceptable acid-addition salt thereof.

A further particular preferred VEGF receptor tyrosine kinase inhibitor for use in the invention is 7-(3-(4-acetylpiperazin-1-yl)propoxy)4-(4-fluoro-2-methylindol-5-yloxy)-6-methoxyquinazoline, or a pharmaceutically-acceptable acid-addition salt thereof.

A further particular preferred VEGF receptor tyrosine kinase inhibitor for use in the invention is 7-[2-(4-acetylpiperazin-1-yl)ethoxy]-4-[(4-fluoro-2-methyl-1H-indol-5-yl)yl)oxy]-6-methoxyquinazoline, or a pharmaceutically-acceptable acid-addition salt thereof.

Particular selective Src kinase inhibitors that may be used for chronic administration in the present invention are described in, for example, International Patent Applications WO 01/94341, WO 02/16352, WO 02/085895, WO 02/092577, WO 02/092578 and WO 02/092579 and in co-pending International Application PCT/GB03/04703 (arising from European Patent Application No. 02292736.2).

Particular Src kinase inhibitors include the following compounds from International Patent Application WO 01/94341:
4-(2-chloro-5-methoxyanilino)-5,7-di-(3-morpholinopropoxy)quinazoline,
4-(2-bromo-5-methoxyanilino)-7-methoxy-5-(N-Methylpiperidin-4-yloxy)quinazoline,
4-(2-chloro-5-methoxyanilino)-7-methoxy-5-N-methylpiperidin-4-yloxy)quinazoline,
4-(2-chloro-5-methoxyanilino)-7-[3-(4-methylpiperazin-1-yl)propoxy]-5-tetrahydropyran-4-yloxyquinazoline,
4-(2-chloro-5-methoxyanilino)-7-(3-morpholinopropoxy)-5-tetrahydropyran-4-yloxyquinazoline,
4-(2-chloro-5-methoxyanilino)-7-[2-hydroxy-3-(4-methylpiperazin-1-yl)propoxy]-5-tetrahydropyran-4-yloxyquinazoline,
4-(2-chloro-5-methoxyanilino)-7-(2-hydroxy-3-morpholinopropoxy)-5-tetrahydropyran-4-yloxyquinazoline,
4-(2-chloro-5-methoxyanilino)-7-[3-(4-methylpiperazin-1-yl)propoxy]-5-tetrahydrofuran-3-yloxyquinazoline,
4-(2-chloro-5-methoxyanilino)-7-(3-morpholinopropoxy)-5-tetrahydrofuran-3-yloxyquinazoline,
4-(5-chloronaphth-1-ylamino)-7-methoxy-5-(N-Methylpiperidin-4-yloxy)quinazoline,
4-(3-chlorobenzofuran-7-ylamino)-7-methoxy-5-(N-Methylpiperidin-4-yloxy)quinazoline,
7-benzyloxy-4-(2-bromo-5-methoxyanilino)-5-piperidin-4-yloxyquinazoline,
4-(2-bromo-5-methoxyanilino)-7-(3-methylsulphonylpropoxy)-5-piperidin-4-yloxyquinazoline,
4-(2-bromo-5-methoxyanilino)-7-methoxy-5-piperidin-4-ylmethoxyquinazoline,
4-(2,4-dichloro-5-methoxyanilino)-7-methoxy-5-(N-Methylpiperidin-4-yloxy)quinazoline,
4-(2,5-dimethoxyanilino)-7-methoxy-5-(N-Methylpiperidin-4-yloxy)quinazoline,
4-(2,4-dichloro-5-methoxyanilino)-7-(2-pyrrolidin-1-ylethoxy)-5-tetrahydropyran-4-yloxyquinazoline,
4-(2,4-dichloro-5-methoxyanilino)-7-(2-piperidinoethoxy)-5-tetrahydropyran-4-yloxyquinazoline,
4-(2,4-dichloro-5-methoxyanilino)-7-(2-morpholinoethoxy)-5-tetrahydropyran-4-yloxyquinazoline,
4-(2,4-dichloro-5-methoxyanilino)-7-[2-(4-methylpiperazin-1-yl)ethoxy]-5-tetrahydropyran-4-yloxyquinazoline,
4-(2-bromo-5-methoxyanilino)-7-(2-pyrrolidin-1-ylethoxy)-5-tetrahydropyran-4-yloxyquinazoline,
4-(2-bromo-5-methoxyanilino)-7-(2-piperidinoethoxy)-5-tetrahydropyran-4-yloxyquinazoline,
4-(2-bromo-5-methoxyanilino)-7-[2-(4-methylpiperazin-1-yl)ethoxy]-5-tetrahydropyran-4-yloxyquinazoline,
4-(2-bromo-5-methoxyanilino)-7-(4-pyridyloxyethoxy)-5-tetrahydropyran-4-yloxyquinazoline,
4-(2-bromo-5-methoxyanilino)-7-{2-[(2S)-2-(N,N-dimethylcarbamoyl)pyrrolidin-1-yl]ethoxy}-5-tetrahydropyran-4-yloxyquinazoline,
4-(2-bromo-5-methoxyanilino)-7-{2-[(2S)-2-(N-Methylcarbamoyl)pyrrolidin-1-yl]ethoxy}-5-tetrahydropyran-4-yloxyquinazoline,
4-(2-bromo-5-methoxyanilino)-7-(4-pyridylmethoxy)-5-tetrahydropyran-4-yloxyquinazoline,
4-(5-methoxy-2-pyrrolidin-1-ylanilino)-7-[3-(4-methylpiperazin-1-yl)propoxy]-5-tetrahydropyran-4-yloxyquinazoline,
4-(2-bromo-5-methoxyanilino)-5-cyclopentyloxy-7-(2-pyrrolidin-1-ylethoxy)quinazoline,
4-(6-chloro-2,3-methylenedioxyanilino)-5-cyclopentyloxy-7-(2-pyrrolidin-1-ylethoxy)quinazoline,
4-(6-chloro-2,3-methylenedioxyanilino)-5-piperidin-4-yloxyquinazoline,
4-(6-chloro-2,3-methylenedioxyanilino)-7-methoxy-5-piperidin-4-yloxyquinazoline,
4-(6-chloro-2,3-methylenedioxyanilino)-7-methoxy-5-(N-methylpiperidin-4-yloxy)quinazoline,
4-(6-chloro-2,3-methylenedioxyanilino)-7-methoxy-5-piperidin-4-ylmethoxyquinazoline,
4-(6-chloro-2,3-methylenedioxyanilino)-7-(2-pyrrolidin-1-ylethoxy)-5-tetrahydropyran-4-yloxyquinazoline,
4-(6-chloro-2,3-methylenedioxyanilino)-7-(3-pyrrolidin-l1-ylpropoxy)-5-tetrahydropyran-4-yloxyquinazoline,
4-(6-chloro-2,3-methylenedioxyanilino)-7-[3-(4-methylpiperazin-1-yl)propoxy]-5-tetrahydropyran-4-yloxyquinazoline,
4-(6-chloro-2,3-methylenedioxyanilino)-7-[2-(4-methylpiperazin-1-yl)ethoxy]-5-tetrahydropyran-4-yloxyquinazoline,
4-(6-chloro-2,3-methylenedioxyanilino)-7-(2-piperidinoethoxy)-5-tetrahydropyran-4-yloxyquinazoline,
4-(6-chloro-2,3-methylenedioxyanilino)-7-[2-(4-pyridyloxy)ethoxy]-5-tetrahydropyran-4-yloxyquinazoline,
4-(6-chloro-2,3-methylenedioxyanilino)-7-piperidin-4-ylmethoxy-5-tetrahydropyran-4-yloxyquinazoline and
4-(6-chloro-2,3-methylenedioxyanilino)-7-(N-Methylpiperidin-4-ylmethoxy)-5-tetrahydropyran-4-yloxyquinazoline;
or a pharmaceutically-acceptable acid-addition salt thereof.

Further particular Src kinase inhibitors include the following compounds from International Patent Application WO 02/16352:
6-methoxy-4-(2,3-methylenedioxyanilino)-7-(3-morpholinopropoxy)quinazoline,
6-methoxy-4-(2,3-methylenedioxyanilino)-7-[3-(1,1-dioxotetrahydro-4H-1,4-thiazin-4-yl)propoxy]quinazoline,
6-methoxy-4-(2,3-methylenedioxyanilino)-7-(3-pyrrolidin-1-ylpropoxy)quinazoline,
6-methoxy-4-(2,3-methylenedioxyanilino)-7-[2-(4-methylpiperazin-1-yl)ethoxy]quinazoline,
6-methoxy-4-(2,3-methylenedioxyanilino)-7-[3-(4-methylpiperazin-1-yl)propoxy]quinazoline,
6-methoxy4-(2,3-methylenedioxyanilino)-7-(3-piperidinopropoxy)quinazoline,
6-methoxy-4-(2,3-methylenedioxyanilino)-7-(N-Methylpiperidin-4-ylmethoxy)quinazoline,
7-(2-hydroxy-3-pyrrolidin-1-ylpropoxy)-6-methoxy-4-(2,3-methylenedioxyanilino)-quinazoline,
7-[2-hydroxy-3-(N-isopropyl-N-methylamino)propoxy]-6-methoxy-4-(2,3-methylenedioxyanilino)quinazoline,
7-[3-(4-cyanomethylpiperazin-1-yl)-2-hydroxypropoxy]-6-methoxy-4-(2,3-methylenedioxyanilino)quinazoline,
6-methoxy-4-(2,3-methylenedioxyanilino)-7-{2-[2-(4-methylpiperazin-1-yl)ethoxy]ethoxy}quinazoline,
4-(6-chloro-2,3-methylenedioxyanilino)-7-[3-(4-cyanomethylpiperazin-1-yl)propoxy]-6-methoxyquinazoline,
4-(6-chloro-2,3-methylenedioxyanilino)-6-methoxy-7-(3-pyrrolidin-1-ylpropoxy)quinazoline,
4-(6-chloro-2,3-methylenedioxyanilino)-6-methoxy-7-(3-piperidinopropoxy)quinazoline,
4-(6-bromo-2,3-methylenedioxyanilino)-6-methoxy-7-(3-piperidinopropoxy)quinazoline,
6-methoxy-4-(2,3-methylenedioxyanilino)-7-[2-(-methylpiperidin-4-yl)ethoxy]quinazoline,
6-methoxy-4-(2,3-methylenedioxyanilino)-7-[2-(4-pyridyloxy)ethoxy]quinazoline,
6-methoxy-4-(2,3-methylenedioxyanilino)-7-(3-pyridylmethoxy)quinazoline,
4-(6-chloro-2,3-methylenedioxyanilino)-7-(2-cyanopyrid-4-ylmethoxy)-6-methoxyquinazoline and
4-(6-chloro-2,3-methylenedioxyanilino)-6-methoxy-7-(N-Methylpiperidin-4-ylmethoxy)quinazoline;
or a pharmaceutically-acceptable acid-addition salt thereof.

Further particular Src kinase inhibitors include the following compounds from International Patent Application WO 02/085895:
6-methoxy-4-(2,3-methylenedioxyphenoxy)-7-(3-pyrrolidin-1-ylpropoxy)quinazoline,
4-(6-chloro-2,3-methylenedioxyphenoxy)-6-methoxy-7-(3-pyrrolidin-1-ylpropoxy)quinazoline,
4-(6-bromo-2,3-methylenedioxyphenoxy)-6-methoxy-7-(3-pyrrolidin-1-ylpropoxy)quinazoline,
6-methoxy-4-(2,3-methylenedioxyphenoxy)-7-(3-morpholinopropoxy)quinazoline,
4-(6-chloro-2,3-methylenedioxyphenoxy)-6-methoxy-7-(3-morpholinopropoxy)quinazoline,
4-(6-bromo-2,3-methylenedioxyphenoxy)-6-methoxy-7-(3-morpholinopropoxy)quinazoline,
6-methoxy-4-(2,3-methylenedioxyphenoxy)-7-[3-(4-methylpiperazin-1-yl)propoxy]quinazoline,
4-(6-chloro-2,3-methylenedioxyphenoxy)-6-methoxy-7-[3-(4-methylpiperazin-1-yl)propoxy]quinazoline,
4-(6-bromo-2,3-methylenedioxyphenoxy)-6-methoxy-7-[3-(4-methylpiperazin-1-yl)propoxy]quinazoline,
6-methoxy-4-(2,3-methylenedioxyphenoxy)-7-(3-methylsulphonylpropoxy)quinazoline,
4-(6-chloro-2,3-methylenedioxyphenoxy)-6-methoxy-7-(3-methylsulphonylpropoxy)quinazoline and
4-(6-bromo-2,3-methylenedioxyphenoxy)-6-methoxy-7-(3-methylsulphonylpropoxy)quinazoline;
or a pharmaceutically-acceptable acid-addition salt thereof.

Further particular Src kinase inhibitors include the following compounds from International Patent Application WO 02/092577:
4-(2-chloro-5-methoxyanilino)-6-methoxy-7-(N-Methylpiperidin-4-ylmethoxy)quinazoline,
4-(2-chloro-5-methoxyanilino)-6-methoxy-7-piperidin-4-ylmethoxyquinazoline and
4-(2-bromo-5-methoxyanilino)-6-methoxy-7-[2-(N-methylpiperidin-4-yl)ethoxy]quinazoline;
or a pharmaceutically-acceptable acid-addition salt thereof.

Further particular Src kinase inhibitors include the following compounds from International Patent Application WO 02/092578:
4-(2,4-dichloro-5-methoxyanilino)-6-methoxy-7-(N-Methylpiperidin-4-ylmethoxy)quinazoline,
4-(2,4-dichloro-5-methoxyanilino)-6-methoxy-7-piperidin-4-ylmethoxyquinazoline,
4-(2,4-dichloro-5-methoxyanilino)-6-methoxy-7-[2-(N-Methylpiperidin-4-yl)ethoxy]quinazoline and
4-(2,4-dichloro-5-methoxyanilino)-6-methoxy-7-(2-piperidin-4-ylethoxy)quinazoline;
or a pharmaceutically-acceptable acid-addition salt thereof.

Further particular Src kinase inhibitors include the following compounds from International Patent Application WO 02/092579:
4-(2-chloro-5-methoxyanilino)-6-methoxy-7-[3-(4-methylpiperazin-1-yl)propoxy]quinazoline,
4-(2-chloro-5-methoxyanilino)-6-methoxy-7-(2-piperidinoethoxy)quinazoline and
4-(2-chloro-5-methoxyanilino)-6-methoxy-7-(2-morpholinoethoxy)quinazoline and
4-(2-bromo-5-methoxyanilino)-6-methoxy-7-[3-(4-methylpiperazin-1-yl)propoxy]quinazoline
or a pharmaceutically-acceptable acid-addition salt thereof.

Further particular Src kinase inhibitors include the following compounds from co-pending International Application PCT/GB03/04703 (arising from European Patent Application No. 02292736.2):
4-(5-chloro-2,3-methylenedioxypyrd-4-ylamino)-6-methoxy-7-[3-(4-prop-2-ynylpiperazin-1-yl)propoxy]quinazoline,
4-(5-chloro-2,3-methylenedioxypyrid-4-ylamino)-7-[3-(4-isobutyrylpiperazin-1-yl)propoxy]-6-methoxyquinazoline,
4-(5-chloro-2,3-methylenedioxypyrid-4-ylamino)-6-methoxy-7-{3-[4-(2,2,2-trifluoroethyl)piperazin-1-yl]propoxy}quinazoline,
4-(5-chloro-2,3-methylenedioxypyrid-4-ylamino)-6-methoxy-7-[2-(4-prop-2-ynylpiperazin-1-yl)ethoxy]quinazoline,
7-[2-(4-acetylpiperazin-1-yl)ethoxy]4-(5-chloro-2,3-methylenedioxypyrid-4-ylamino)-5-tetrahydropyran-4-yloxyquinazoline,
4-(5-chloro-2,3-methylenedioxypyrid-4-ylamino)-7-{2-[(3RS,4SR)-3,4-methylenedioxypyrrolidin-1-yl)ethoxy]-5-tetrahydropyran-4-yloxyquinazoline,
7-[2-(4-acetylpiperazin-1-yl)ethoxy]-4-(5-chloro-2,3-methylenedioxypyrid-4-ylamino)-5-isopropoxyquinazoline and
4-(5-chloro-2,3-methylenedioxypyrid-4-ylamino)-7-{2-[(3RS,4SR)-3,4-methylenedioxypyrrolidin-1-yl)ethoxy]-5-isopropoxyquinazoline;
or a pharmaceutically-acceptable acid-addition salt thereof.

More particular selective Src kinase inhibitors include the following compounds:
4-(2,4-dichloro-5-methoxyanilino)-7-(2-piperidinoethoxy)-5-tetrahydropyran-4-yloxyquinazoline,
4-(2,4-dichloro-5-methoxyanilino)-7-(2-morpholinoethoxy)-5-tetrahydropyran-4-yloxyquinazoline,
4-(2,4-dichloro-5-methoxyanilino)-7-[2-(4-methylpiperazin-1-yl)ethoxy]-5-tetrahydropyran-4-yloxyquinazoline,
4-(2-bromo-5-methoxyanilino)-7-(2-pyrrolidin-1-ylethoxy)-5-tetrahydropyran-4-yloxyquinazoline,
4-(6-chloro-2,3-methylenedioxyanilino)-7-(2-pyrrolidin-1-ylethoxy)-5-tetrahydropyran-4-yloxyquinazoline,
4-(6-chloro-2,3-methylenedioxyanilino)-7-(3-pyrrolidin-1-ylpropoxy)-5-tetrahydropyran-4-yloxyquinazoline,
4-(6-chloro-2,3-methylenedioxyanilino)-7-[3-(4-methylpiperazin-1-yl)propoxy]-5-tetrahydropyran-4-yloxyquinazoline,
4-(6-chloro-2,3-methylenedioxyanilino)-7-[2-(4-methylpiperazin-1-yl)ethoxy]-5-tetrahydropyran-4-yloxyquinazoline,
4-(6-chloro-2,3-methylenedioxyanilino)-7-(2-piperidinoethoxy)-5-tetrahydropyran-4-yloxyquinazoline,
7-[2-(4-acetylpiperazin-1-yl)ethoxy]-4-(6-chloro-2,3-methylenedioxyanilino)-5-isopropoxyquinazoline,
6-methoxy-4-(2,3-methylenedioxyanilino)-7-(3-morpholinopropoxy)quinazoline,
6-methoxy-4-(2,3-methylenedioxyanilino)-7-[3-(1,1-dioxotetrahydro-4H-1,4-thiazin-4-yl)propoxy]quinazoline,
6-methoxy-4-(2,3-methylenedioxyanilino)-7-(3-pyrrolidin-1-ylpropoxy)quinazoline,
6-methoxy-4-(2,3-methylenedioxyanilino)-7-[2-(4-methylpiperazin-1-yl)ethoxy]quinazoline,
6-methoxy-4-(2,3-methylenedioxyanilino)-7-[3-(4-methylpiperazin-1-yl)propoxy]quinazoline,
6-methoxy-4-(2,3-methylenedioxyanilino)-7-(3-piperidinopropoxy)quinazoline,
4-(6-chloro-2,3-methylenedioxyanilino)-7-[3-(4-isobutyrylpiperazin-1-yl)propoxy]-6-methoxyquinazoline,
4-(2-chloro-5-methoxyanilino)-6-methoxy-7-(N-Methylpiperidin-4-ylmethoxy)quinazoline,
4-(2-chloro-5-methoxyanilino)-6-methoxy-7-piperidin-4-ylmethoxyquinazoline,
4-(2-bromo-5-methoxyanilino)-6-methoxy-7-[2-(N-methylpiperidin-4-yl)ethoxy]quinazoline,
4-(2,4-dichloro-5-methoxyanilino)-6-methoxy-7-(N-Methylpiperidin-4-ylmethoxy)quinazoline,
4-(2,4-dichloro-5-methoxyanilino)-6-methoxy-7-piperidin-4-ylmethoxyquinazoline,
4-(2,4-dichloro-5-methoxyanilino)-6-methoxy-7-[2-(N-methylpiperidin-4-yl)ethoxy]quinazoline,
4-(5-chloro-2,3-methylenedioxypyrid-4-ylamino)-6-methoxy-7-[3-(4-prop-2-ynylpiperazin-1-yl)propoxy]quinazoline,
7-[2-(4-acetylpiperazin-1-yl)ethoxy]-4-(5-chloro-2,3-methylenedioxypyrid-4-ylamino)-5-tetrahydropyran-4-yloxyquinazoline,
4-(5-chloro-2,3-methylenedioxypyrid-4-ylamino)-7-{2-[(3RS,4SR)-3,4-methylenedioxypyrrolidin-1-yl)ethoxy]-5-tetrahydropyran-4-yloxyquinazoline,
7-[2-(4-acetylpiperazin-1-yl)ethoxy]4-(5-chloro-2,3-methylenedioxypyrid-4-ylamino)-5-isopropoxyquinazoline and
4-(5-chloro-2,3-methylenedioxypyrid-4-ylamino)-7-{2-[(3RS,4SR)-3,4-methylenedioxypyrrolidin-1-yl)ethoxy]-5-isopropoxyquinazoline;
or a pharmaceutically-acceptable acid-addition salt thereof.

Preferred selective Src kinase inhibitors include the following compounds:
4-(2,4-dichloro-5-methoxyanilino)-7-(2-piperidinoethoxy)-5-tetrahydropyran-4-yloxyquinazoline,
4-(2,4-dichloro-5-methoxyanilino)-7-(2-morpholinoethoxy)-5-tetrahydropyran-4-yloxyquinazoline,
4-(6-chloro-2,3-methylenedioxyanilino)-7-(2-pyrrolidin-1-ylethoxy)-5-tetrahydropyran-4-yloxyquinazoline,
4-(6-chloro-2,3-methylenedioxyanilino)-7-(3-pyrrolidin-1-ylpropoxy)-5-tetrahydropyran-4-yloxyquinazoline,
4-(6-chloro-2,3-methylenedioxyanilino)-7-[2-(4-methylpiperazin-1-yl)ethoxy]-5-tetrahydropyran-4-yloxyquinazoline,
4-(6-chloro-2,3-methylenedioxyanilino)-7-(2-piperidinoethoxy)-5-tetrahydropyran-4-yloxyquinazoline,
7-[2-(4-acetylpiperazin-1-yl)ethoxy]-4-(6-chloro-2,3-methylenedioxyanilino)-5-isopropoxyquinazoline,
6-methoxy4-(2,3-methylenedioxyanilino)-7-(3-morpholinopropoxy)quinazoline,
6-methoxy4(2,3-methylenedioxyanilino)-7-(3-pyrrolidin-1-ylpropoxy)quinazoline,
6-methoxy-4-(2,3-methylenedioxyanilino)-7-[3-(4-methylpiperazin-1-yl)propoxy]quinazoline,
6-methoxy-4-(2,3-methylenedioxyanilino)-7-(3-piperidinopropoxy)quinazoline,
4-(6-chloro-2,3-methylenedioxyanilino)-7-[3-(4-isobutyrylpiperazin-1-yl)propoxy]-6-methoxyquinazoline,
4-(2-chloro-5-methoxyanilino)-6-methoxy-7-(N-Methylpiperidin-4-ylmethoxy)quinazoline,
4-(2-chloro-5-methoxyanilino)-6-methoxy-7-piperidin-4-ylmethoxyquinazoline,
4-(2,4-dichloro-5-methoxyanilino)-6-methoxy-7-N-methylpiperidin-4-ylmethoxy)quinazoline,
4-(2,4-dichloro-5-methoxyanilino)-6-methoxy-7-piperidin-4-ylmethoxyquinazoline,
7-[2-(4-acetylpiperazin-1-yl)ethoxy]-4-(5-chloro-2,3-methylenedioxypyrid-4-ylamino)-5-tetrahydropyran-4-yloxyquinazoline,
4-(5-chloro-2,3-methylenedioxypyrid-4-ylamino)-7-{2-[(3RS,4SR)-3,4-methylenedioxypyrrolidin-1-yl)ethoxy]-5-tetrahydropyran-4-yloxyquinazoline,
7-[2-(4-acetylpiperazin-1-yl)ethoxy]-4-(5-chloro-2,3-methylenedioxypyrid-4-ylamino)-5-isopropoxyquinazoline and
4-(5-chloro-2,3-methylenedioxypyrid-4-ylamino)-7-{2-[(3RS,4SR)-3,4-methylenedioxypyrrolidin-1-yl)ethoxy]-5-isopropoxyquinazoline;
or a pharmaceutically-acceptable acid-addition salt thereof.

A particular preferred Src kinase inhibitor for use in the invention is 4-(6-chloro-2,3-methylenedioxyanilino)-7-(2-pyrrolidin-1-ylethoxy)-5-tetrahydropyran-4-yloxyquinazoline, or a pharmaceutically-acceptable acid-addition salt thereof.

A further particular preferred Src kinase inhibitor for use in the invention is 4-(6-chloro-2,3-methylenedioxyanilino)-7-(3-pyrrolidin-1-ylpropoxy)-5-tetrahydropyran-4-yloxyquinazoline, or a pharmaceutically-acceptable acid-addition salt thereof.

A further particular preferred Src kinase inhibitor for use in the invention is 4-(6-chloro-2,3-methylenedioxyanilino)-7-[2-(4-methylpiperazin-1-yl)ethoxy]-5-tetrahydropyran-4-yloxyquinazoline, or a pharmaceutically-acceptable acid-addition salt thereof.

A further particular preferred Src kinase inhibitor for use in the invention is 4-(6-chloro-2,3-methylenedioxyanilino)-7-(2-piperidinoethoxy)-5-tetrahydropyran-4-yloxyquinazoline, or a pharmaceutically-acceptable acid-addition salt thereof.

A further particular preferred Src kinase inhibitor for use in the invention is 7-[2-(4-acetylpiperazin-1-yl)ethoxy]-4-(6-chloro-2,3-methylenedioxyanilino)-5-isopropoxyquinazoline, or a pharmaceutically-acceptable acid-addition salt thereof.

A further particular preferred Src kinase inhibitor for use in the invention is 6-methoxy-4-(2,3-methylenedioxyanilino)-7-(3-morpholinopropoxy)quinazoline, or a pharmaceutically-acceptable acid-addition salt thereof.

A further particular preferred Src kinase inhibitor for use in the invention is 6-methoxy-4-(2,3-methylenedioxyanilino)-7-(3-piperidinopropoxy)quinazoline, or a pharmaceutically-acceptable acid-addition salt thereof.

A further particular preferred Src kinase inhibitor for use in the invention is 4-(6-chloro-2,3-methylenedioxyanilino)-7-[3-(4-isobutyrylpiperazin-1-yl)propoxyl-6-methoxyquinazoline, or a pharmaceutically-acceptable acid-addition salt thereof.

A further particular preferred Src kinase inhibitor for use in the invention is 4-(2-chloro-5-methoxyanilino)-6-methoxy-7-N-methylpiperidin-4-ylmethoxy)quinazoline, or a pharmaceutically-acceptable acid-addition salt thereof.

A further particular preferred Src kinase inhibitor for use in the invention is 7-[2-(4-acetylpiperazin-1-yl)ethoxy]-4-(5-chloro-2,3-methylenedioxypyrid-4-ylamino)-5-tetrahydropyran-4-yloxyquinazoline, or a pharmaceutically-acceptable acid-addition salt thereof.

A further particular preferred Src kinase inhibitor for use in the invention is 4-(5-chloro-2,3-methylenedioxypyrid-4-ylamino)-7-{2-[(3RS,4SR)-3,4-methylenedioxypyrrolidin-1-yl)ethoxy]-5-tetrahydropyran-4-yloxyquinazoline, or a pharmaceutically-acceptable acid-addition salt thereof.

A further particular preferred Src kinase inhibitor for use in the invention is 7-[2-(4-acetylpiperazin-1-yl)ethoxy]-4-(5-chloro-2,3-methylenedioxypyrid-4-ylamino)-5-isopropoxyquinazoline, or a pharmaceutically-acceptable acid-addition salt thereof.

A further particular preferred Src kinase inhibitor for use in the invention is 4-(5-chloro-2,3-methylenedioxypyrid-4-ylamino)-7-{2-[(3RS,4SR)-3,4-methylenedioxypyrrolidin-1-yl)ethoxy]-5-isopropoxyquinazoline, or a pharmaceutically-acceptable acid-addition salt thereof.

A suitable pharmaceutically-acceptable salt of those VEGF receptor tyrosine kinase inhibitors as defined hereinbefore or those Src kinase inhibitors as defined hereinbefore that are sufficiently basic is, for example, a pharmaceutically-acceptable acid-addition salt, for example an acid-addition salt with an inorganic or organic acid such as hydrochloric, hydrobromic, sulphuric, trifluoroacetic, citric or maleic acid. A suitable pharmaceutically-acceptable salt of those VEGF receptor tyrosine kinase inhibitors as defined hereinbefore or those Src kinase inhibitors as defined hereinbefore that are sufficiently acidic is, for example, a pharmaceutically-acceptable alkali or alkaline earth metal salt such as a calcium or magnesium salt, or an ammonium salt, or a salt with an organic base such as methylamine, dimethylamine, trimethylamine, piperidine, morpholine or tris-(2-hydroxyethyl)amine.

In order to use a VEGF receptor tyrosine kinase inhibitor as defined hereinbefore or a Src kinase inhibitor as defined hereinbefore according to the present invention, the compounds may be administered using suitable pharmaceutical compositions. For example, a composition may be in a form suitable for oral administration, for example as a tablet or capsule, for parenteral injection (including intravenous, subcutaneous, intramuscular, intravascular or infusion) for example as a sterile solution, suspension or emulsion, for topical administration for example as an ointment or cream, for rectal administration for example as a suppository or the route of administration may be by direct injection into the tumour or by regional delivery or by local delivery. In other embodiments of the present invention the components may be delivered endoscopically, intratracheally, intralesionally, percutaneously, intravenously, subcutaneously or intraperitoneally. In general the compositions described herein may be prepared in a conventional manner using conventional excipients or carriers that are well known in the art.

Suitable pharmaceutically-acceptable excipients or carriers for a tablet formulation include, for example, inert excipients such as lactose, sodium carbonate, calcium phosphate or calcium carbonate, granulating and disintegrating agents such as corn starch or alginic acid, binding agents such as gelatin or starch, lubricating agents such as magnesium stearate, stearic acid or talc, preservative agents such as ethyl or propyl 4-hydroxybenzoate, and anti-oxidants such as ascorbic acid. Tablet formulations may be uncoated or coated either to modify their disintegration and the subsequent absorption of the active ingredient within the gastrointestinal tract or to improve their stability and/or appearance, in either case using conventional coating agents and procedures well known in the art.

Compositions for oral use may be in the form of hard gelatin capsules in which the active ingredient is mixed with an inert solid excipient, for example, calcium carbonate, calcium phosphate or kaolin, or as soft gelatin capsules in which the active ingredient is mixed with water or an oil such as peanut oil, liquid paraffin or olive oil.

Suitable compositions may be obtained by conventional procedures using conventional pharmaceutical excipients, well known in the art. Thus, compositions intended for oral use may contain, for example, one or more colouring, sweetening, flavouring and/or preservative agents.

As stated hereinbefore, it is to be understood that the term a “combination” envisages the simultaneous, separate or sequential administration of the components of the combination. It will be appreciated that the pharmaceutical composition according to the present invention includes a pharmaceutical composition comprising an anti-angiogenic agent and a Src kinase inhibitor as defined hereinbefore and a pharmaceutically-acceptable excipient or carrier. Such a composition conveniently provides the components of the combination for simultaneous administration. A pharmaceutical composition according to the present invention also includes separate compositions comprising a first composition comprising an anti-angiogenic agent and a pharmaceutically-acceptable excipient or carrier, and a second composition comprising a Src kinase inhibitor and a pharmaceutically-acceptable excipient or carrier. Such a composition conveniently provides the components of the combination for sequential or separate administration but the separate compositions may also be administered simultaneously. Conveniently such a pharmaceutical composition of the invention comprises a kit comprising a first container with a suitable composition containing the anti-angiogenic agent and a second container with a suitable composition containing the Src kinase inhibitor.

According to this aspect of the present invention there is provided a kit for use in dosing the combination defined hereinbefore comprising:

a) an anti-angiogenic agent together with a pharmaceutically-acceptable excipient or carrier, in a first unit dosage form;

b) a Src kinase inhibitor together with a pharmaceutically-acceptable excipient or carrier, in a second unit dosage form; and

c) container means for containing said first and second dosage forms; and characterised in that an appropriate dose of each component of the combination is selected such that the contrasting blood pressure effects associated with the individual use of either component of the combination are substantially counter-balanced.

According to a further aspect of the present invention, there is provided a synergistic combination product comprising an anti-angiogenic agent as defined herein and a Src kinase inhibitor as defined herein for use simultaneously, sequentially or separately in the production of an anti-cancer effect in a warm-blooded animal such as a human being.

It is to be understood that, according to this aspect of the present invention, a combination product is defined as affording a synergistic effect if the effect is therapeutically superior to that achievable on dosing one or other of the components of the combination treatment, as measured by, for example, the extent of the response, the response rate, the time to disease progression or the survival period. For example, the effect of the combination product is synergistic if the effect is therapeutically superior to the effect achievable with an anti-angiogenic agent or a Src kinase inhibitor alone. Further, the effect of the combination product is synergistic if a beneficial effect is obtained in a group of patients that does not respond (or responds poorly) to an anti-angiogenic agent or a Src kinase inhibitor alone. Further, the effect of the combination product is synegistic if a beneficial effect is obtained but with fewer and/or less troublesome side-effects than those that may occur if conventional doses of each component are used.

It is to be understood that there is no requirement that the anti-angiogenic and Src kinase inhibitor components of the combination product must be dosed simultaneously. Sequential or separate use of these components may also provide the desired beneficial effect and such administration is to be understood to fall within the ambit of this aspect of the present invention. Thus, this aspect of the invention envisages simultaneous administration of the anti-angiogenic agent and the Src kinase inhibitor. This aspect of the invention also envisages sequential administration of those agents. This aspect also envisages separate administration of those agents. Where the administration of those agents is sequential or separate, the delay in administering the second component should not be such as to lose the benefit of a synergistic anti-cancer effect.

According to a further aspect of the present invention, there is provided a blood pressure effect sparing combination product comprising an anti-angiogenic agent as defined herein and a Src kinase inhibitor as defined herein for use simultaneously, sequentially or separately as defined hereinbefore in the production of an anti-cancer effect in a warm-blooded animal such as a human being.

This aspect of the present invention relates to ways in which an anti-cancer effect, especially an anti-tumour effect, for example that based in part on the anti-angiogenic effect of a VEGF receptor tyrosine kinase inhibitor, may be produced in a warm-blooded animal such as a human being without causing the hypertension that is associated with the use of an anti-angiogenic agent.

Hypertension is a prevalent cardiovascular disorder that affects many millions of people and, despite the availability of several classes of anti-hypertensive agents, cardiovascular disease remains an important cause of patient morbidity and mortality. Accordingly, it may be useful to counter the sustained increase in blood pressure that occurs when an anti-angiogenic agent such as a VEGF receptor tyrosine kinase inhibitor is administered. According to this aspect of the present invention, such an effect is achieved by also administering a Src kinase inhibitor. The resultant combination product has a substantially sparing effect on blood pressure changes. Accordingly, this aspect of the invention provides a blood pressure sparing combination product.

The combination product as defined hereinbefore requires that an appropriate dose of each component of the combination product is selected such that the contrasting blood pressure effects associated with the individual use of either component of the combination product are substantially counter-balanced. In one embodiment of the present invention, a first component of the combination product is dosed at its conventional dose and the second component is dosed in an amount that substantially counter-balances the blood pressure effect associated with the individual use of the first component. Blood pressure effects are measured by conventional means. Thereby the anti-cancer effect is maintained or improved as measured by one or more of the extent of the response, the response rate, the time to disease progression and survival data, in particular the duration of the response. In another embodiment of the present invention, the conventional dose of the first component of the combination product may be reduced and the second component is dosed in an amount that substantially counter-balances the blood pressure effect associated with the individual use of the first component and the anti-cancer effect is maintained or improved as measured by one or more of the extent of the response, the response rate, the time to disease progression and survival data, in particular the duration of the response. Thereby the anti-cancer effect is maintained or improved but with fewer and/or less troublesome side-effects than those that may occur if conventional doses of each component are used.

As stated hereinbefore, anti-angiogenic agents that possess pharmacokinetic properties which provide a reasonable bioavailability when administered chronically lead to an increase in diastolic blood pressure in the rat of about 10 to 30 mm Hg and in human beings of about 10 to 20 mm Hg. Src kinase inhibitors that possess pharmacokinetic properties which provide a reasonable bioavailability after a single dose lead to a decrease in diastolic blood pressure in the rat of about 10 to 25 mm Hg. It will be appreciated that the contrasting blood pressure effects associated with the individual use of either of an anti-angiogenic agent or of a Src kinase inhibitor will be substantially counter-balanced if the Src kinase inhibition reduces the hypertensive effect of the anti-angiogenic agent on diastolic blood pressure to less than about 10 mm Hg, particularly to less than about 5 mm Hg. Further, a blood pressure sparing effect will be substantially achieved if the resultant diastolic blood pressure effect of appropriate doses of a combination product of an anti-angiogenic agent and a Src kinase inhibitor is in the range of about −10 to +10 mm Hg, particularly in the range of about −5 to +5 mm Hg. More particularly, a blood pressure sparing effect will be substantially achieved if an approximately normotensive effect is achieved.

Subject to that blood pressure sparing effect, an anti-angiogenic agent as defined hereinbefore will generally be administered chronically so that a daily dose in the range, for example, 0.01 mg/kg to 50 mg/kg body weight is received, given if required in divided doses. In general lower doses will be administered when a parenteral route is employed. Thus, for example, for intravenous administration, a daily dose in the range, for example, 0.01 mg/kg to 25 mg/kg body weight will generally be used. Similarly, for administration by inhalation, a daily dose in the range, for example, 0.01 mg/kg to 25 mg/kg body weight will be used. Oral administration is however preferred, particularly in tablet form, to provide a daily dose in the range, for example, 0.01 mg/kg to 10 mg/kg body weight, conveniently 0.01 mg/kg to 5 mg/kg body weight.

Subject to that blood pressure sparing effect, a Src kinase inhibitor as defined hereinbefore will generally be administered chronically so that a daily dose in the range, for example, 0.02 mg/kg to 75 mg/kg body weight is received, given if required in divided doses. In general lower doses will be administered when a parenteral route is employed. Thus, for example, for intravenous administration, a daily dose in the range, for example, 0.01 mg/kg to 30 mg/kg body weight will generally be used. Similarly, for administration by inhalation, a daily dose in the range, for example, 0.01 mg/kg to 25 mg/kg body weight will be used. Oral administration is however preferred, particularly in tablet form, to provide a daily dose in the range, for example, 0.02 mg/kg to 15 mg/kg body weight, conveniently 0.02 mg/kg to 5 mg/kg body weight.

According to a preferred version of this aspect of the present invention there is provided a combination product comprising an anti-angiogenic agent as defined hereinbefore and a Src kinase inhibitor as defined hereinbefore for use simultaneously, sequentially or separately as defined hereinbefore in the production of a substantially normotensive anti-cancer effect in a warm-blooded mammal such as a human being.

According to a preferred version of this aspect of the present invention there is provided a combination product comprising an anti-angiogenic agent as defined hereinbefore and a Src kinase inhibitor as defined hereinbefore for use simultaneously, sequentially or separately as defined hereinbefore in the production of an anti-cancer effect in a warm-blooded mammal such as a human being characterised in that an appropriate dose of each component of the combination product is selected such that the contrasting blood pressure effects associated with the individual use of either component of the combination product are substantially counter-balanced.

According to a preferred version of this aspect of the present invention there is provided a combination product comprising an anti-angiogenic agent selected from:
4-(4-fluoro-2-methylindol-5-yloxy)-6-methoxy-7-(3-(pyrrolidin-1-yl)propoxy)quinazoline,
4-(4-fluoro-2-methylindol-5-yloxy)-6-methoxy-7-(3-piperidinopropoxy)quinazoline,
4-(4-fluoro-2-methylindol-5-yloxy)-6-methoxy-7-(3-(4-methylpiperazin-1-yl)propoxy)quinazoline,
4-(4-fluoro-2-methylindol-5-yloxy)-6-methoxy-7-(2-(1-methylpiperidin-4-yl)ethoxy]quinazoline,
4-(4-chloro-2-fluoroanilino)-6-methoxy-7-(N-methylpiperidin-4-ylmethoxy)quinazoline,
4-(4-bromo-2-fluoroanilino)-6-methoxy-7-(l -methylpiperidin-4-ylmethoxy)quinazoline,
4-(4-bromo-2-fluoroanilino)-6-methoxy-7-(piperidin-4-ylmethoxy)quinazoline,
4-[(4-fluoro-2-methylindol-5-yl)oxy]-6-methoxy-7-{3-[4-(2-propynyl)piperazin-1-yl]propoxy}quinazoline,
7-(3-(4-acetylpiperazin-1-yl)propoxy)-4-(4-fluoro-2-methylindol-5-yloxy)-6-methoxyquinazoline and
7-[2-(4-acetylpiperazin-1-yl)ethoxy]-4-[(4-fluoro-2-methyl-1H-indol-5-yl)oxy]-6-methoxyquinazoline,
or a pharmaceutically-acceptable acid-addition salt thereof;

and a Src kinase inhibitor selected from:
4-(6-chloro-2,3-methylenedioxyanilino)-7-(2-pyrrolidin-1-ylethoxy)-5-tetrahydropyran-4-yloxyquinazoline,
4-(6-chloro-2,3-methylenedioxyanilino)-7-(3-pyrrolidin-1-ylpropoxy)-5-tetrahydropyran-4-yloxyquinazoline,
4-(6-chloro-2,3-methylenedioxyanilino)-7-[2-(4-methylpiperazin-1-yl)ethoxy]-5-tetrahydropyran-4-yloxyquinazoline,
4-(6-chloro-2,3-methylenedioxyanilino)-7-(2-piperidinoethoxy)-5-tetrahydropyran-4-yloxyquinazoline,
7-[2-(4-acetylpiperazin-1-yl)ethoxy]-4-(6-chloro-2,3-methylenedioxyanilino)-5-isopropoxyquinazoline,
6-methoxy-4-(2,3-methylenedioxyanilino)-7-(3-morpholinopropoxy)quinazoline,
6-methoxy-4-(2,3-methylenedioxyanilino)-7-(3-piperidinopropoxy)quinazoline,
4-(6-chloro-2,3-methylenedioxyanilino)-7-[3-(4-isobutyrylpiperazin-1-yl)propoxy]-6-methoxyquinazoline,
4-(2-chloro-5-methoxyanilino)-6-methoxy-7-(N-methylpiperidin-4-ylmethoxy)quinazoline,
7-[2-(4-acetylpiperazin-1-yl)ethoxy]-4-(5-chloro-2,3-methylenedioxypyrid-4-ylamino)-5-tetrahydropyran-4-yloxyquinazoline,
4-(5-chloro-2,3-methylenedioxypyrid-4-ylamino)-7-{2-[(3RS,4SR)-3,4-methylenedioxypyrrolidin-1-yl)ethoxy]-5-tetrahydropyran-4-yloxyquinazoline,
7-[2-(4-acetylpiperazin-1-yl)ethoxy)-4-(5-chloro-2,3-methylenedioxypyrid-4-ylamino)-5-isopropoxyquinazoline and
4-(5-chloro-2,3-methylenedioxypyrid-4-ylamino)-7-{2-[(3RS,4SR)-3,4-methylenedioxypyrrolidin-1-yl)ethoxy]-5-isopropoxyquinazoline,
or a pharmaceutically-acceptable acid-addition salt thereof;
   for use simultaneously, sequentially or separately as defined hereinbefore in the production of a substantially normotensive anti-cancer effect in a warm-blooded mammal such as a human being or for use in the production of an anti-cancer effect in a warm-blooded mammal such as a human being characterised in that an appropriate dose of each component of the combination product is selected such that the contrasting blood pressure effects associated with the individual use of either component of the combination product are substantially counter-balanced.

The combination product of this aspect of the present invention may be administered in the form of a suitable pharmaceutical composition as defined hereinbefore. According to this aspect of the invention there is provided a pharmaceutical composition for use in the production of an anti-tumour effect in a warm-blooded mammal such as a human being which comprises a combination product as defined hereinbefore in association with a pharmaceutically-acceptable excipient or carrier.

Whilst taking account of the fact that an appropriate dose of each component of the combination product is selected such that the contrasting blood pressure effects associated with the individual use of either component of the combination product are substantially counter-balanced (i.e. that a substantially normotensive effect is obtained), the amount of active ingredient that is combined with one or more excipients to produce a single dosage form will necessarily vary depending upon the host treated and the particular route of administration.

Subject to that counter-balancing need, an anti-angiogenic agent as defined hereinbefore will generally be administered chronically so that a daily dose in the range, for example, 0.01 mg/kg to 50 mg/kg body weight is received, given if required in divided doses. In general lower doses will be administered when a parenteral route is employed. Thus, for example, for intravenous administration, a daily dose in the range, for example, 0.01 mg/kg to 25 mg/kg body weight will generally be used. Similarly, for administration by inhalation, a daily dose in the range, for example, 0.01 mg/kg to 25 mg/kg body weight will be used. Oral administration is however preferred, particularly in tablet form, to provide a daily dose in the range, for example, 0.01 mg/kg to 10 mg/kg body weight, conveniently 0.01 mg/kg to 5 mg/kg body weight.

Subject to that counter-balancing need, a Src kinase inhibitor as defined hereinbefore will generally be administered chronically so that a daily dose in the range, for example, 0.02 mg/kg to 75 mg/kg body weight is received, given if required in divided doses. In general lower doses will be administered when a parenteral route is employed. Thus, for example, for intravenous administration, a daily dose in the range, for example, 0.01 mg/kg to 30 mg/kg body weight will generally be used. Similarly, for administration by inhalation, a daily dose in the range, for example, 0.01 mg/kg to 25 mg/kg body weight will be used. Oral administration is however preferred, particularly in tablet form, to provide a daily dose in the range, for example, 0.02 mg/kg to 15 mg/kg body weight, conveniently 0.02 mg/kg to 5 mg/kg body weight.

It will be appreciated that the pharmaceutical composition according to the present invention includes a pharmaceutical composition comprising a combination product as defined hereinbefore (comprising an anti-angiogenic agent and a Src kinase inhibitor) and a pharmaceutically-acceptable excipient or carrier. Such a composition conveniently provides the combination product of the invention for simultaneous administration.

A pharmaceutical composition according to this aspect of the present invention also includes separate compositions comprising a first composition comprising an anti-angiogenic agent and a pharmaceutically-acceptable excipient or carrier, and a second composition comprising a Src kinase inhibitor and a pharmaceutically-acceptable excipient or carrier. Such a composition conveniently provides the combination product of the invention as defined hereinbefore for sequential or separate administration but the separate compositions may also be administered simultaneously. Conveniently such a pharmaceutical composition of the invention comprises a kit comprising a first container with a suitable composition containing the anti-angiogenic agent and a second container with a suitable composition containing the Src kinase inhibitor.

According to this aspect of the present invention there is provided a kit for use in dosing a combination product as defined hereinbefore to produce an anti-cancer effect in a warm-blooded mammal such as a human being comprising:

a) an anti-angiogenic agent together with a pharmaceutically-acceptable excipient or carrier in a first unit dosage form;

b) a Src kinase inhibitor together with a pharmaceutically-acceptable excipient or carrier in a second unit dosage form; and

c) container means for containing said first and second dosage forms.

According to a further preferred version of this aspect of the present invention there is also provided a combination product comprising the anti-angiogenic agent 4-(4-fluoro-2-methylindol-5-yloxy)-6-methoxy-7-(3-piperidinopropoxy)quinazoline, or a pharmaceutically-acceptable acid-addition salt thereof, and the Src kinase inhibitor 7-[2-(4-acetylpiperazin-1-yl)ethoxy]-4-(6-chloro-2,3-methylenedioxyanilino)-5-isopropoxyquinazoline, or a pharmaceutically-acceptable acid-addition salt thereof.

According to a further preferred version of this aspect of the present invention there is also provided a combination product comprising the anti-angiogenic agent 4-(4-fluoro-2-methylindol-5-yloxy)-6-methoxy-7-(3-piperidinopropoxy)quinazoline, or a pharmaceutically-acceptable acid-addition salt thereof, and the Src kinase inhibitor 4-(6-chloro-2,3-methylenedioxyanilino)-7-[2-(4-methylpiperazin-1-yl)ethoxy]-5-tetrahydropyran-4-yloxyquinazoline, or a pharmaceutically-acceptable acid-addition salt thereof.

According to a further preferred version of this aspect of the present invention there is also provided a combination product comprising the anti-angiogenic agent 4-(4-fluoro-2-methylindol-5-yloxy)-6-methoxy-7-(3-piperidinopropoxy)quinazoline, or a pharmaceutically-acceptable acid-addition salt thereof, and the Src kinase inhibitor 7-[2-(4-acetylpiperazin-1-yl)ethoxy]-4-(5-chloro-2,3-methylenedioxypyrid-4-ylamino)-5-isopropoxyquinazoline, or a pharmaceutically-acceptable acid-addition salt thereof.

According to a further preferred version of this aspect of the present invention there is also provided a combination product comprising the anti-angiogenic agent 4-(4-bromo-2-fluoroanilino)-6-methoxy-7-(1-methylpiperidin-4-ylmethoxy)quinazoline, or a pharmaceutically-acceptable acid-addition salt thereof, and the Src kinase inhibitor 7-[2-(4-acetylpiperazin-1-yl)ethoxy]-4-(6-chloro-2,3-methylenedioxyanilino)-5-isopropoxyquinazoline, or a pharmaceutically-acceptable acid-addition salt thereof.

According to a further preferred version of this aspect of the present invention there is also provided a combination product comprising the anti-angiogenic agent 4-(4-bromo-2-fluoroanilino)-6-methoxy-7-(1-methylpiperidin-4-ylmethoxy)quinazoline, or a pharmaceutically-acceptable acid-addition salt thereof, and the Src kinase inhibitor 4-(6-chloro-2,3-methylenedioxyanilino)-7-[2-(4-methylpiperazin-1-yl)ethoxy]-5-tetrahydropyran-4-yloxyquinazoline, or a pharmaceutically-acceptable acid-addition salt thereof.

According to a further preferred version of this aspect of the present invention there is also provided a combination product comprising the anti-angiogenic agent 4-(4-bromo-2-fluoroanilino)-6-methoxy-7-(1-methylpiperidin-4-ylmethoxy)quinazoline, or a pharmaceutically-acceptable acid-addition salt thereof, and the Src kinase inhibitor 7-[2-(4-acetylpiperazin-1-yl)ethoxy]-4-(5-chloro-2,3-methylenedioxypyrid-4-ylamino)-5-isopropoxyquinazoline, or a pharmaceutically-acceptable acid-addition salt thereof.

According to a further preferred version of this aspect of the present invention there is also provided a combination product comprising the anti-angiogenic agent 4-(4-fluoro-2-methylindol-5-yloxy)-6-methoxy-7-(3-(pyrrolidin-1-yl)propoxy)quinazoline, or a pharmaceutically-acceptable acid-addition salt thereof, and the Src kinase inhibitor 7-[2-(4-acetylpiperazin-1-yl)ethoxy]-4-(6-chloro-2,3-methylenedioxyanilino)-5-isopropoxyquinazoline, or a pharmaceutically-acceptable acid-addition salt thereof.

According to a further preferred version of this aspect of the present invention there is also provided a combination product comprising the anti-angiogenic agent 4-(4-fluoro-2-methylindol-5-yloxy)-6-methoxy-7-(3-(pyrrolidin-1-yl)propoxy)quinazoline, or a pharmaceutically-acceptable acid-addition salt thereof, and the Src kinase inhibitor 4-(6-chloro-2,3-methylenedioxyanilino)-7-[2-(4-methylpiperazin-1-yl)ethoxy]-5-tetrahydropyran-4-yloxyquinazoline, or a pharmaceutically-acceptable acid-addition salt thereof.

According to a further preferred version of this aspect of the present invention there is also provided a combination product comprising the anti-angiogenic agent 4-(4-fluoro-2-methylindol-5-yloxy)-6-methoxy-7-(3-(pyrrolidin-1-yl)propoxy)quinazoline, or a pharmaceutically-acceptable acid-addition salt thereof, and the Src kinase inhibitor 7-[2-(4-acetylpiperazin-1-yl)ethoxy]4-(5-chloro-2,3-methylenedioxypyrid-4-ylamino)-5-isopropoxyquinazoline, or a pharmaceutically-acceptable acid-addition salt thereof.

According to a further preferred version of this aspect of the present invention there is also provided a combination product comprising the anti-angiogenic agent 7-(3-(4-acetylpiperazin-1-yl)propoxy)4-(4-fluoro-2-methylindol-5-yloxy)-6-methoxyquinazoline, or a pharmaceutically-acceptable acid-addition salt thereof, and the Src kinase inhibitor 7-[2-(4-acetylpiperazin-1-yl)ethoxy]-4-(6-chloro-2,3-methylenedioxyanilino)-5-isopropoxyquinazoline, or a pharmaceutically-acceptable acid-addition salt thereof.

According to a further preferred version of this aspect of the present invention there is also provided a combination product comprising the anti-angiogenic agent 7-(3-(4-acetylpiperazin-1-yl)propoxy)-4-(4-fluoro-2-methylindol-5-yloxy)-6-methoxyquinazoline, or a pharmaceutically-acceptable acid-addition salt thereof, and the Src kinase inhibitor 4-(6-chloro-2,3-methylenedioxyanilino)-7-[2-(4-methylpiperazin-1-yl)ethoxy]-5-tetrahydropyran-4-yloxyquinazoline, or a pharmaceutically-acceptable acid-addition salt thereof.

According to a further preferred version of this aspect of the present invention there is also provided a combination product comprising the anti-angiogenic agent 7-(3-(4-acetylpiperazin-1-yl)propoxy)4-(4-fluoro-2-methylindol-5-yloxy)-6-methoxyquinazoline, or a pharmaceutically-acceptable acid-addition salt thereof, and the Src kinase inhibitor 7-[2-(4-acetylpiperazin-1-yl)ethoxy]4-(5-chloro-2,3-methylenedioxypyrid-4-ylamino)-5-isopropoxyquinazoline, or a pharmaceutically-acceptable acid-addition salt thereof.

According to a further aspect of the present invention there is provided the use of a combination product as defined hereinbefore in the manufacture of a medicament for use in the substantially normotensive production of an anti-cancer effect in a warm-blooded mammal such as a human being, the Src kinase inhibitor being administered in an amount effective to counteract substantially the hypertension induced by the anti-angiogenic agent (or for use in the production of an anti-cancer effect in a warm-blooded mammal such as a human being characterised in that an appropriate dose of each component of the combination product is selected such that the contrasting blood pressure effects associated with the individual use of either component of the combination product are substantially counter-balanced).

According to a further feature of the present invention there is provided a method for the substantially normotensive production of an anti-cancer effect in a warm-blooded mammal such as a human being which comprises the administration of an effective amount of an anti-angiogenic agent in combination with a Src kinase inhibitor, said Src kinase inhibitor being administered in an amount effective to counteract substantially the hypertension induced by said anti-angiogenic agent.

According to a further feature of the present invention there is provided a method for the production of an anti-cancer effect in a warm-blooded mammal such as a human being which comprises the administration of effective amounts of the components of the combination product as defined hereinbefore characterised in that an appropriate dose of each component of the combination product is selected such that the contrasting blood pressure effects associated with the individual use of either component of the combination product are substantially counter-balanced.

According to a further feature of the present invention there is provided a method for the production of an anti-cancer effect in a warm-blooded mammal such as a human being which comprises the simultaneous, sequential or separate administration to a warm-blooded mammal such as a human being that is in need of such treatment of effective amounts of the components of the combination product as defined hereinbefore characterised in that an appropriate dose of each component of the combination product is selected such that the contrasting blood pressure effects associated with the individual use of either component of the combination product are substantially counter-balanced.

### EXAMPLE

Measurement of Blood Pressure in Conscious Rats by Radio-Telemetry

Blood pressure was measured using commercially-available radio-telemetry equipment (Data Sciences International, Saint Paul, Minn., USA) which provides a means for the remote measurement of the blood pressure (BP), heart rate and activity of a conscious, unrestrained laboratory animal such as a rat. Measurements obtained using this system have the advantage that the test animal is free from stresses induced by surgery and/or restraint.

The equipment comprises a pressure transducer (Code No. TA11PA-C40; hereinafter the ‘pressure transducer implant’) that is implanted into the abdomen of a laboratory rat. The transducer transmits a radio signal indicating the pressure in the aorta of the animal and the signal is detected by a receiver (RA1010) placed under the plastic cage which houses the animal. The signal is recorded and evaluated automatically by pre-written computer software (DataQuest 2.1 that may be installed on a suitable computer such as an IBM-compatible personal computer containing an Intel™ 486 processor).

Implantation Methodology

Each of a group of normotensive rats (Alderley Park strain, male animals) was anaesthetised with “Fluothane™” inhalation anaesthetic. The abdomen of each rat was shaved and the skin was coated with a topical disinfectant. An incision was made in the outer skin to expose the abdominal muscle wall which was cut along the mid-line and opened. The viscera of the animal was held back with retractors and the abdominal aorta was located. The aorta was cleaned of connective tissue over a 2-3 cm length and carefully separated from the associated vena cava. Care was taken to ensure that the area of aorta prepared was below the renal arteries to avoid any potential occlusion of the kidneys following surgery. The tip of a 21 gauge needle (Micro Lance, Becton Dickinson) was bent to approximately 90 degrees to the needle shaft. A tie was placed loosely under the aorta. The tie was lifted to occlude the blood vessel and the needle was used to form a puncture into the blood vessel. With the needle held in place in the blood vessel, the bevel of the needle was used carefully to control the insertion of the tip of the catheter from the ‘pressure transducer implant’ into the blood vessel. The needle tip was withdrawn and a small drop of surgical glue (Vet Bond 3H) was run down the catheter to form a seal between the catheter and the blood vessel. A cellulose patch was placed over the seal to stabilise the catheter. The ‘pressure transducer implant’ was stitched into position on the inside of the abdominal wall and the abdominal muscle wall was closed with absorbable stitches. The ends of the stitches were trimmed and the outer skin of the animal was closed using surgical autoclips which were removed 7 days after surgery.

General Study Protocol

The animals were housed in a facility using a 12 hour cycle of light and dark. Normal rat behaviour was seen during the Studies i.e. the animals rested during the light phase and were active during the dark phase. Following removal of the surgical autoclips, all rats were handled daily and dosed daily with control vehicle (citrate buffer or 1% polysorbate 80 in water) for a further week in order to acclimatise them to dosing techniques. Blood pressure data were recorded from each animal every 10 minutes throughout each Study. To obtain more reproducible basal blood pressure measurements, data were obtained during the 12 hour light phase when the test animals were inactive.

Typically, on day 1, each of a group of 3 rats was dosed p.o. at approximately 9.00 am with control vehicle and blood pressure data were recorded during the ensuing 24 hour period. The following day, each rat was dosed p.o. at approximately 9.00 am with a suitable dose of a test compound or with a combination of test compounds and blood pressure data were recorded during the ensuing 24 hour period. Doses were selected that provided sufficient blood levels of the test compounds that a sustained effect on blood pressure was obtained indicating that an anti-tumour effect would be obtainable in an appropriate animal model. The difference was calculated between the basal blood pressure on day 1 and the basal blood pressure on day 2 following the dosing of the test compound or combination of test compounds. For single agent Studies, both the maximum effect on blood pressure (in mm Hg compared to blood pressure data in control animals) and the time (in hours) for the restoration of normotension were recorded. For combination Studies, the selected doses of each compound were co-administered and adjusted if necessary to obtain a substantially normotensive effect. Illustrative results are shown in the Figures hereinafter wherein:

VTK-1 is the compound 4-(4fluoro-2-methylindol-5-yloxy)-6-methoxy-7-(3-piperidinopropoxy)quinazoline which provides Example 238 of International Patent Application WO 00/47212 and

Src-1 is the compound 7-[2-(4-acetylpiperazin-1-yl)ethoxy]4-(6-chloro-2,3-methylenedioxyanilino)-5-isopropoxyquinazoline which may be prepared as described hereinafter.

### BRIEF DESCRIPTION OF THE DRAWINGS

FIG. 1 shows the diastolic blood pressure profile following the single dose p.o. at about 9.00 am of control citrate buffer vehicle (thinner line) or of 1.5 mg/kg of VTK-1 (thicker line) with time (minutes) plotted on the horizontal axis and diastolic blood pressure (mm Hg) plotted on the vertical axis.

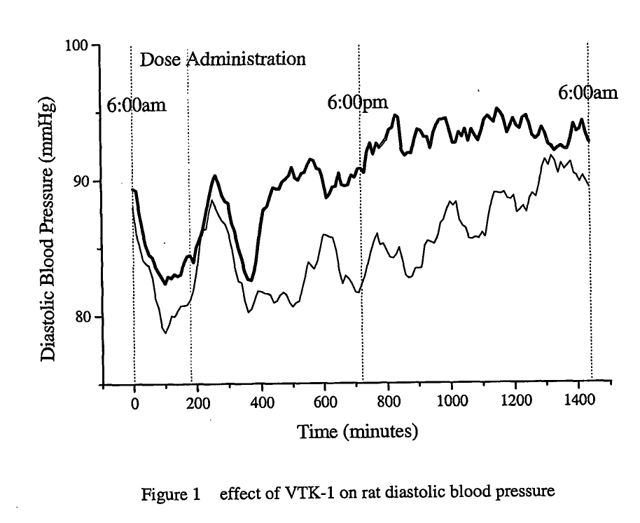

FIG. 2 shows the diastolic blood pressure profile following the single dose p.o. at about 9.00 am of control citrate buffer vehicle (thinner line) or of 25 mg/kg of Src-1 (thicker line) with time (minutes) plotted on the horizontal axis and diastolic blood pressure (mm Hg) plotted on the vertical axis.

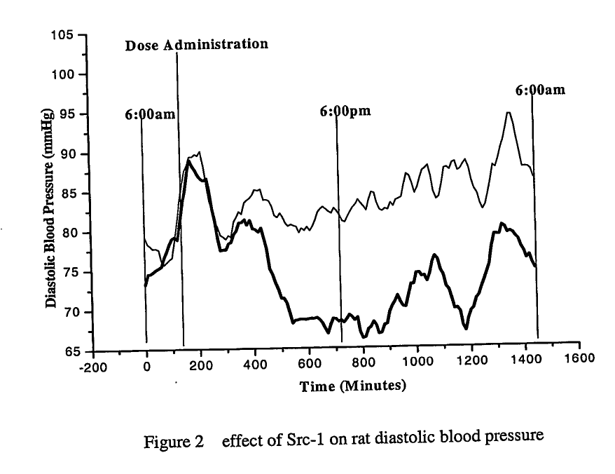

FIG. 3 shows the diastolic blood pressure profile following the single dose p.o. at about 9.00 am of control citrate buffer vehicle (thinner line) or of a combination of 1.5 mg/kg of VTK-1 and 25 mg/kg of Src-1 (thicker line) with time (minutes) plotted on the horizontal axis and diastolic blood pressure (mm Hg) plotted on the vertical axis.

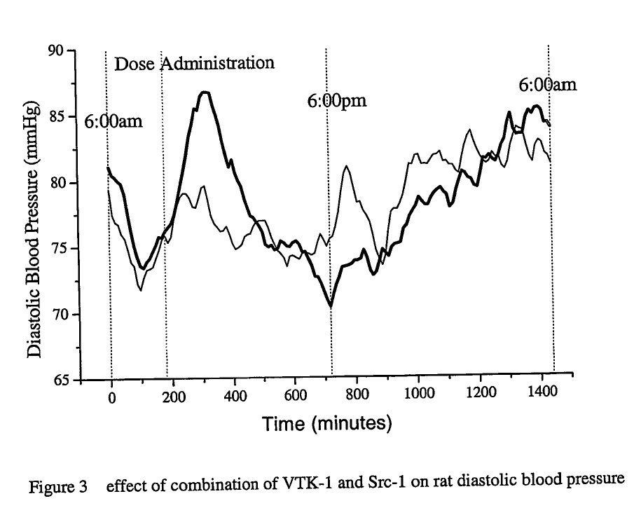

The data in the Figures show that the contrasting blood pressure effects of the anti-angiogenic agent VTK-1 and the Src kinase inhibitor Src-1 can be substantially counter-balanced.

In general, in the following Examples:

(i) operations were carried out at ambient temperature, i.e. in the range 17 to 25° C. and under an atmosphere of an inert gas such as argon unless otherwise stated;

(ii) evaporations were carried out by rotary evaporation in vacuo and work-up procedures were carried out after removal of residual solids by filtration;

(iii) column chromatography (by the flash procedure) and medium pressure liquid chromatography (MPLC) were performed on Merck Kieselgel silica (Art. 9385) or Merck Lichroprep RP-18 (Art. 9303) reversed-phase silica obtained from E. Merck, Darmstadt, Germany or high pressure liquid chromatography (BPLC) was performed on C18 reverse phase silica, for example on a Dynamax C-18 60 Å preparative reversed-phase column;

(iv) yields, where present, are not necessarily the maximum attainable;

(v) in general, the end-products have satisfactory microanalyses and their structures were confirmed by nuclear magnetic resonance (NMR) and/or mass spectral techniques; fast-atom bombardment (FAB) mass spectral data were obtained using a Platform spectrometer and, where appropriate, either positive ion data or negative ion data were collected; NMR chemical shift values were measured on the delta scale [proton magnetic resonance spectra were determined using a Jeol JNM EX 400 spectrometer operating at a field strength of 400 MHz, Varian Gemini 2000 spectrometer operating at a field strength of 300 MHz or a Bruker AM300 spectrometer operating at a field strength of 300 MHz]; the following abbreviations have been used: s, singlet; d, doublet; t, triplet; q, quartet; m, multiplet; br, broad;

(vi) intermediates were not generally fully characterised and purity was assessed by thin layer chromatographic, HPLC, infra-red (IR) and/or NMR analysis;

(vii) melting points are uncorrected and were determined using a Mettler SP62 automatic melting point apparatus or an oil-bath apparatus; melting points for the end-products of the Formula I were determined after crystallisation from a conventional organic solvent such as ethanol, methanol, acetone, ether or hexane, alone or in admixture;

(viii) where certain compounds were obtained as an acid-addition salt, for example a mono hydrochloride salt or a dihydrochloride salt, the stoichiometry of the salt was based on the number and nature of the basic groups in the compound, the exact stoichiometry of the salt was generally not determined, for example by means of elemental analysis data;

(ix) the following abbreviations have been used:
DMF N,N-dimethylfornamide
DMSO dimethylsulphoxide
THF tetrahydrofuran
DMA N,N-dimethylacetarnide

### Example: Preparation of Src-1

### 7-[2-(4-acetylpiperazin-1-yl)ethoxy]-4-(6-chloro-2,3-methylenedioxyanilino)-5-isopropoxyquinazoline

A mixture of 7-(2-chloroethoxy)-4-(6-chloro-2,3-methylenedioxyanilino)-5-isopropoxyquinazoline (3.39 g), 1-acetylpiperazine (3 g), potassium iodide (2.57 g) and DMA (40 ml) was stirred and heated to 95° C. for 3 hours. The mixture was cooled and the solvent was evaporated. The residue was partitioned between methylene chloride and a 5% aqueous sodium bicarbonate solution. The organic phase was dried over magnesium sulphate and evaporated. The residue was purified by column chromatography on silica using increasingly polar mixtures of methylene chloride and methanol as eluent. The residue was triturated under diethyl ether. There was thus obtained the title compound as a crystalline solid (3.49 g); NMR Spectrum: (CDCl₃) 1.5 (s, 3H), 1.51 (s, 3H), 2.08 (s, 3H), 2.55 (m, 4H), 2.86 (t, 2H), 3.5 (m, 2H), 3.67 (m, 2H), 4.21 (t, 2H), 4.8 (m, 1H), 6.03 (s, 2H), 6.5 (s, 1H), 6.69 (d, 1H), 6.79 (s, 1H), 6.94 (d, 1H), 8.49 (s, 1H), 9.39 (s, 1H); Mass Spectrum: M+H⁺ 528; Elemental Analysis Found: C, 59.2; H, 6.0; N, 13.1; Cl, 6.7; C₂₆H₃₀ClN₅O₅ requires C, 59.1; H, 5.7; N, 13.3; Cl, 6.7%.

The 7-(2-chloroethoxy)-4-(6-chloro-2,3-methylenedioxyanilino)-5-isopropoxyquinazoline used as a starting material was prepared as follows:

Di-tert-butyl azodicarboxylate (28.9 g) was added to a stirred mixture of 7-benzyloxy-5-hydroxy-3-pivaloyloxymethyl-3,4-dihydroquinazolin-4-one (International Patent Application WO 01/94341, Example 15, Note [8] thereof; 30 g), isopropanol (7.3 ml), triphenylphosphine (32.95 g) and methylene chloride (350 ml) that had been cooled to 0° C. The reaction mixture was allowed to warm to ambient temperature and was stirred for 1.5 hours. The mixture was evaporated and the residue was purified by column chromatography on silica using increasingly polar mixtures of methylene chloride and methanol as eluent. There was thus obtained 7-benzyloxy-5-isopropoxy-3,4-dihydroquinazolin-4-one as a solid (23.8 g); NMR Spectrum: (DMSOd₆) 7.89 (s, 1H), 7.5-7.3 (m, 5H), 6.75 (s, 1H), 6.62 (s, 1H), 5.24 (s, 2H), 4.65 (m, 1H), 1.29 (d, 6H).

Ammonium formate (48.4 g) was added to a stirred mixture of 7-benzyloxy-5-isopropoxy-3,4-dihydroquinazolin-4-one (23.8 g), 10% palladium-on-carbon catalyst (2.8 g) and DMF (300 ml) and the resultant mixture was stirred at ambient temperature for 2 hours. The mixture was filtered and the filtrate was evaporated. The material so obtained was triturated under water, the pH of which was adjusted to pH7. The solid so obtained was collected by filtration, washed with water and with diethyl ether and dried over phosphorus pentoxide under vacuum. There was thus obtained 7-hydroxy-5-isopropoxy-3,4-dihydroquinazolin-4-one as a white solid (15.9 g); NMR Spectrum: (DMSOd₆) 1.3 (d, 6H), 4.57 (m, 1H), 6.42 (s, 1H), 6.5 (s, 1H), 7.8 (s, 1H).

A mixture of the material so obtained, acetic anhydride (34 ml) and pyridine (0.62 ml) was heated to 70° C. for 30 minutes. The reaction mixture was cooled to ambient temperature and the excess of acetic anhydride was evaporated. The white solid so obtained was added to hot water (80° C., 250 ml) and the mixture was stirred vigorously and heated to 80° C. for 20 minutes. The mixture was cooled to ambient temperature and the solid was isolated and dried over phosphorus pentoxide. There was thus obtained 7-acetoxy-5-isopropoxy-3,4-dihydroquinazolin-4-one (17.86 g); NMR Spectrum: (DMSOd₆) 7.97 (s, 1H), 6.91 (s, 1H), 6.85 (s, 1H), 4.65 (m, 1H), 2.32 (s, 3H), 1.33 (d, 6H).

Phosphorus oxychloride (2.13 ml) was added to a mixture of 7-acetoxy-5-isopropoxy-3,4-dihydroquinazolin-4-one (5 g), N,N-diisopropyl-N-ethylamine (8.62 ml) and 1,2-dichloroethane (140 ml) and the mixture was heated to 75° C. for 2.5 hours. The mixture was cooled to ambient temperature and the solvent was evaporated in vacuo to give 7-acetoxy-4-chloro-5-isopropoxyquinazoline which was used without further purification.

A mixture of the material so obtained, 6-chloro-2,3-methylenedioxyaniline (3.27 g; International Patent Application WO 01/94341, Example 17, Note [30]) and isopropanol (45 ml) was stirred and heated to 80° C. for 1 hour. The resultant mixture was cooled to ambient temperature and the sovents were evaporated. The residue was partitioned between methylene chloride and a 10% aqueous ammonium hydroxide solution. The organic solution was washed with brine, dried over magnesium sulphate and evaporated. The residue was dissolved in methylene chloride (45 ml) and a 7N solution of ammonia in methanol (45 m1l) was added and the mixture was stirred at ambient temperature for 1 hour. After evaporation of the solvents, the residue was purified by column chromatography on silica using initially ethyl acetate and then a 10:1 mixture of methylene chloride and methanol as eluent. There was thus obtained 4-(6-chloro-2,3-methylenedioxyanilino)-7-hydroxy-5-isopropoxyquinazoline as a white solid (3.79 g); NMR Spectrum: (DMSOd₆) 1.45 (s, 3H), 1.46 (s, 3H), 4.93 (m, 1H), 6.08 (s, 2H), 6.67 (m, 2H), 6.9 (d, 1H), 7.07 (d, 1H), 8.28 (s, 1H), 9.28 (s, 1H).

A mixture of the material so obtained, 1,2-dichloroethane (55 ml) and potassium carbonate (2.52 g) was stirred and heated to 80° C. for 24 hours. The mixture was cooled to ambient temperature and the solvent was evaporated. The residue was diluted with methylene chloride and insoluble material was filtered off. The filtrate was evaporated and the residue was purified by column chromatography on silica using a 20:1 mixture of methylene chloride and methanol as eluent. There was thus obtained 7-(2-chloroethoxy)-4-(6-chloro-2,3-methylenedioxyanilino)-5-isopropoxyquinazoline (3.39 g); NMR Spectrum: (CDCl₃) 1.54 (s, 3H), 1.55 (s, 3H), 3.88 (t, 2H), 4.36 (t, 2H), 4.84 (m, 1H), 6.05 (s, 2H), 6.56 (s, 1H), 6.71 (d, 1H), 6.79 (s, 1H), 6.97 (d, 1H), 8.54 (s, 1H), 9.42 (s, 1H); Mass Spectrum: M+H⁺ 436.

Src Inhibitors Described Within International Application PCT/GB03/04703 (Arising from European Patent Application No. 02292736.2)

### Example 1

### 4-(5-chloro-2,3-methylenedioxypyrid-4-ylamino)-7-(3-chloropropoxy)-6-methoxyquinazoline

Sodium hexamethyldisilazane (1M solution in THF; 0.734 ml) was added to a solution of 4-amino-5-chloro-2,3-methylenedioxypyridine (0.12 g) in DME (4 ml) that had been cooled to 0° C. and the mixture was stirred for 15 minutes. A portion (0.1 g) of 4-chloro-7-(3-chloropropoxy)-6-methoxyquinazoline was added and the resultant mixture was stirred and allowed to warm to ambient temperature. The mixture was stirred at ambient temperature for 16 hours. The reaction mixture was evaporated and the residue was partitioned between methylene chloride and a saturated aqueous ammonium chloride solution. The organic phase was washed with water and with brine, dried over magnesium sulphate and evaporated. The residue was purified by column chromatography on silica using increasingly polar mixtures of methylene chloride and ethyl acetate as eluent followed by increasingly polar mixtures of methylene chloride and acetonitrile. There was thus obtained the title compound as a white foam (0.11 g); NMR Spectrum: (DMSOd₆ and CD₃CO₂D) 2.3 (m, 2H), 3.8 (m, 2H), 4.05 (s, 3H), 4.4 (t, 2H), 6.3 (s, 2H), 7.4 (s, 1H), 7.9 (s, 1H), 8.15 (s, 1H), 8.95 (s, 1H); Mass Spectrum: M+H⁺ 423 and 425.

The 4amino-5-chloro-2,3-methylenedioxypyridine used as a starting material was prepared as follows:

Bromochloromethane (20 ml) was added to a mixture 5-chloro-2,3-dihydroxypyridine (30 g), caesium carbonate (100 g) and DMF (300 ml) and the mixture was stirred and heated to 90° C. for 3.5 hours. The mixture was cooled to ambient temperature and filtered. The filtrate was evaporated and the residue was purified by column chromatography on silica using methylene chloride as eluent. There was thus obtained 5-chloro-2,3-methylenedioxypyridine as a white solid (4.7 g); NMR Spectrum: (DMSOd₆) 6.25 (s, 2H), 7.5 (s, 1H), 7.65 (s, 1H).

A mixture of diisopropylamine (8.2 ml) and THF (100 ml) was cooled to −70° C. and n-butyllithium (2.5 M in hexane, 24 ml) was added dropwise. The mixture was stirred at −70° C. for a further 20 minutes. A solution of 5-chloro-2,3-methylenedioxypyridine (4.2 g) in THF (40 ml) was added over 10 minutes and the reaction mixture was stirred at −70° C. for 1 hour. Dry carbon dioxide gas was bubbled into the reaction mixture for 30 minutes. The resultant reaction mixture was allowed to warm to ambient temperature. Water (20 ml) was added and the organic solvent was evaporated. The residue was acidified to pH2 by the addition of 1N aqueous hydrochloric acid solution. The resultant solid was isolated and washed in turn with water and diethyl ether and dried under vacuum at 40° C. There was thus obtained 5-chloro-2,3-methylenedioxypyridine-4-carboxylic acid (3.6 g); ¹³C NMR Spectrum: (DMSOd₆) 103, 120, 121, 138, 140, 158, 163.

A mixture of the material so obtained, diphenylphosphoryl azide (3.6 ml), anhydrous tert-butanol (13.5 ml), triethylamine (4.2 ml) and 1,4-dioxane (63 ml) was stirred and heated to 100° C. for 3 hours. The mixture was evaporated and the residue was partitioned between ethyl acetate and water. The organic phase was washed with water, dried over magnesium sulphate and evaporated. The residue was purified by column chromatography on silica using a 9:1 mixture of methylene chloride and ethyl acetate as eluent. There was thus obtained tert-butyl 5-chloro-2,3-methylenedioxypyrid-4-ylcarbamate (3.8 g); NMR Spectrum: (DMSOd₆) 1.45 (s, 9H), 6.2 (s, 2H), 7.7 (s, 1H), 9.2 (s, 1H).

The material so obtained was dissolved in methylene chloride (35 ml) and the solution was cooled to 0° C. Trifluoroacetic acid (15 ml) was added and the mixture was stirred at 0° C. for 3 hours. The mixture was allowed to warm to ambient temperature and was stirred for 16 hours. The solvent was evaporated and the residue was diluted with ice water and neutralised to pH7 by the addition of 2N aqueous sodium hydroxide solution whilst keeping the mixture temperature at 0° C. The resultant mixture was extracted with methylene chloride and the extract dried over magnesium sulphate and evaporated. The residue was purified by column chromatography on silica using a 19:1 mixture of methylene chloride and diethyl ether as eluent. There was thus obtained 4-amino-5-chloro-2,3-methylenedioxypyridine (2 g); NMR Spectrum: (DMSOd₆) 6.1 (s, 2H), 6.2 (s, 2H), 7.45 (s, 1H); ¹³C NMR Spectrum: (DMSOd₆) 100, 112, 125, 136, 138, 157; Mass Spectrum: M+H⁺ 173.

The 4-chloro-7-(3-chloropropoxy)-6-methoxyquinazoline used as a starting material was prepared as follows:

Ammonium formate (45 g) was added portionwise over 1.25 hours to a stirred mixture of 7-benzyloxy-6-methoxy-3,4-dihydroquinazolin-4-one (International Patent Application WO 02/16352, Example 1 thereof; 20 g), 10% palladium-on-carbon catalyst (3.3 g) and DMF (530 ml) and the reaction mixture was stirred for an additional 30 minutes. The catalyst was removed by filtration and the solvent was evaporated. There was thus obtained 7-hydroxy-6-methoxy-3,4-dihydroquinazolin-4-one (8.65 g); NMR Spectrum: (DMSOd₆) 3.9 (s, 3H), 7.0 (s, 1H), 7.45 (s, 1H), 7.9 (s, 1H).

A mixture of the material so obtained, acetic anhydride (63 ml) and pyridine (7.5 ml) was heated to 100° C. for 4.5 hours. The resultant mixture was allowed to stand at ambient temperature for 16 hours. The mixture was poured into a stirred mixture (400 ml) of ice and water. The resultant precipitate was isolated and dried under vacuum. Analysis revealed that hydrolysis of the acetate group on the 4-position of the quinazoline was incomplete. The mixture was therefore further hydrolysed with water (150 ml) and pyridine (a few drops) at 90° C. for 15 minutes. The resultant mixture was cooled to ambient temperature and the solid was collected by filtration, washed with water and dried under vacuum. There was thus obtained 7-acetoxy-6-methoxy-3,4-dihydroquinazolin-4-one (7.4 g); NMR Spectrum: (DMSOd₆) 2.3 (s, 3H), 3.9 (s, 3H), 7.45 (s, 1H), 7.65 (s, 1H), 8.05 (s, 1H).

A mixture of a portion (2 g) of the material so obtained, thionyl chloride (32 ml) and DMF (5 drops) was stirred and heated to reflux for 1.5 hours. The mixture was cooled to ambient temperature and the excess of thionyl chloride was evaporated. Toluene was added to the residue and evaporated. The resultant residue was diluted with methylene chloride (15 ml) and a 10:1 mixture (80 ml) of methanol and a saturated aqueous ammonium hydroxide solution was added. The resultant mixture was stirred and heated to 80° C. for 10 minutes. The mixture was cooled to ambient temperature and evaporated. Water was added to the residue and the mixture was neutralised by the addition of dilute aqueous hydrochloric acid solution. The resultant precipitate was collected by filtration and dried under vacuum at 35° C. for 16 hours. There was thus obtained 4chloro-7-hydroxy-6-methoxyquinazoline (1.65 g); NMR Spectrum: DMSOd₆) 4.0 (s, 3H), 7.25 (s, 1H), 7.4 (s, 1H), 8.8 (s, 1H).

Di-tert-butyl azodicarboxylate (2.3 g) was added portionwise over a few minutes to a stirred mixture of 4-chloro-7-hydroxy-6-methoxyquinazoline (1.65 g), 3-chloropropanol (0.7 ml), triphenylphosphine (2.6 g) and methylene chloride (100 ml) and the reaction mixture was stirred at ambient temperature for 2 hours. The mixture was concentrated to a volume of about 30 ml by evaporation and the residue was purified by column chromatography on silica using increasingly polar mixtures of petroleum ether (b.p 40-60° C.) and ethyl acetate as eluent. There was thus obtained 4-chloro-7-(3-chloropropoxy)-6-methoxyquinazoline as a white solid (2 g); NMR Spectrum: (DMSOd₆) 2.3 (m, 2H), 3.8 (m, 2H), 4.05 (s, 3H), 4.4 (m, 2H), 7.45 (s, 1H), 7.55 (s, 1H), 8.9 (s, 1H).

### Example 2

### 7-(2-chloroethoxy)-4-(5-chloro-2,3-methylenedioxypyrid-4-ylamino)-6-methoxyquinazoline

Using an analogous procedure to that described in Example 1,4-chloro-7-(2-chloroethoxy)-6-methoxyquinazoline was reacted with 4-amino-5-chloro-2,3-methylenedioxypyridine to give the title compound in 92% yield; NMR Spectrum: (DMSOd₆ and CD₃CO₂D) 4.05 (s, 3H), 4.1 (t, 2H), 4.55 (t, 2H), 6.3 (s, 2H), 7.4 (s, 1H), 7.9 (s, 1H), 8.15 (s, 1H), 8.95 (s, 1H); Mass Spectrum: M+H⁺ 409 and 411.

The 4-chloro-7-(2-chloroethoxy)-6-methoxyquinazoline used as a starting material was prepared as follows:

1,2-Dichloroethane (400 ml) was added to a stirred mixture of 7-hydroxy-6-methoxy-3-pivaloyloxymethyl-3,4-dihydroquinazolin-4-one (International Patent Application WO 02/16352, Example 2, Note [4] thereof; 85 g), potassium carbonate (77 g) and DMF (400 ml) and the reaction mixture was heated to 70° C. for 16 hours. The reaction mixture was cooled to ambient temperature and filtered. The filtrate was evaporated and the solid so obtained was washed with water and dried over phosphorus pentoxide at 50° C. The material so obtained was purified by column chromatography on silica using increasingly polar mixtures of methylene chloride and ethyl acetate as eluent. There was thus obtained 7-(2-chloroethoxy)-6-methoxy-3-pivaloyloxymethyl-3,4-dihydroquinazolin-4-one as a white solid (65.6 g); NMR Spectrum: (CDCl₃) 1.2 (s, 9H), 3.9 (t, 2H), 4.0 (s, 3H), 4.4 (t, 2H), 5.95 (s, 2H), 7.1 (s, 1H), 7.7 (s, 1H), 8.2 (s, 1H); Mass Spectrum: M+H⁺ 369 and 371.

A mixture of the material so obtained and a saturated solution of ammonia gas in methanol (1.6 L) was stirred at ambient temperature for 2 days. The solvent was concentrated by evaporation to about one-fourth of the original volume and the precipitate was collected by filtration and washed with diethyl ether. There was thus obtained 7-(2-chloroethoxy)-6-methoxy-3,4-dihydroquinazolin-4-one as a white solid (44 g); NMR Spectrum: (DMSOd₆) 3.9 (s, 3H), 4.05 (t, 2H), 4.4 (t, 2H), 7.15 (s, 1H), 7.45 (s, 1H), 8.0 (s, 1H); Mass Spectrum: M+H⁺ 255 and 257.

A mixture of a portion (5 g) of the material so obtained, thionyl chloride (28 ml) and DMF (0.7 ml) was stirred and heated to 80° C. for 1.5 hours. The excess of thionyl chloride was evaporated and toluene was added and evaporated. The residual solid was suspended in a mixture of ice and water and basified to pH7.5 by the addition of 2N aqueous sodium hydroxide solution followed by a saturated aqueous sodium bicarbonate solution. The resultant solid was collected by filtration, washed with water and diethyl ether and dried over over phosphorus pentoxide under vacuum. The material so obtained was purified by column chromatography on silica using increasingly polar mixtures of methylene chloride and acetonitrile as eluent. There was thus obtained 4-chloro-7-(2-chloroethoxy)-6-methoxyquinazoline (3.06 g); NMR Spectrum: (CDCl₃) 3.95 (t, 2H), 4.1 (s, 3H), 4.5 (t, 2H), 7.35 (s, 1H), 7.45 (s, 1H), 8.9 (s, 1); Mass Spectrum: M+H⁺ 273 and 275.

### Example 3

### 4-(5-chloro-2,3-methylenedioxypyrid-4-ylamino)-6-methoxy-7-[3-(4-prop-2-ynylpiperazin-1-yl)propoxylquinazoline

A mixture of 4-(5-chloro-2,3-methylenedioxypyrid-4-ylamino)-7-(3chloropropoxy)-6-methoxyquinazoline (0.08 g), 1-prop-2-ynylpiperazine (0.047 g), potassium iodide (0.01 g) and DMA (2 ml) was stirred and heated to 80° C. for 3.5 hours. The solvent was evaporated and the residue was partitioned between methylene chloride and a saturated aqueous ammonium chloride solution. The organic phase was dried over magnesium sulphate and evaporated. The residue was purified by column chromatography on silica using a 19:1 mixture of methylene chloride and methanol and then a 9:1 mixture of methylene chloride and a saturated methanolic ammonia solution as eluent. The resulting gum was triturated under diethyl ether. There was thus obtained the title compound as a solid (0.066 g); NMR Spectrum: (DMSOd₆ and CF₃CO₂D) 2.3 (m, 2H), 3.2-3.6 (br m, 10H), 3.75 (s, 1H), 3.95 (br s, 2H), 4.0 (s, 3H), 4.35 (m, 2H), 6.3 (s, 2H), 7.4 (s, 1H), 7.9 (s, 1H), 8.15 (s, 1H), 8.95 (s, 1H); Mass Spectrum: M+H⁺ 511 and 513.

The 1-prop-2-ynylpiperazine used as a starting material was prepared as follows

Propargyl bromide (80% solution in toluene; 40 ml) was added dropwise during 10 minutes to a stirred mixture of 1-tert-butoxycarbonylpiperazine (50 g), potassium carbonate (74.2 g) and acetonitrile (2 L) that had been cooled to 0° C. The mixture was stirred for 1.5 hours and allowed to warm to ambient temperature. The mixture was filtered and the filtrate was evaporated. The residue was purified by column chromatography on silica using increasingly polar mixtures of methylene chloride and ethyl acetate as eluent. There was thus obtained tert-butyl 4-prop-2-ynylpiperazine-1-carboxylate as an oil (45.5 g); NMR Spectrum: (CDCl₃) 1.4 (s, 9H), 2.2 (s, 1H), 2.45 (m, 4H), 3.3 (s, 2H), 3.45 (m, 4).

A solution of the material so obtained in methylene chloride (100 ml) was added slowly to a solution of hydrogen chloride gas in 1,4-dioxane (4M, 450 ml). The reaction was slightly exothermic and a precipitate formed as carbon dioxide gas was evolved. The mixture was stirred at ambient temperature for 1 hour. The resultant mixture was evaporated and the residue was suspended in methylene chloride. A solution of ammonia gas in methanol (7M, 110 ml) was added and the mixture was stirred at ambient temperature for 15 minutes. The mixture was filtered and the filtrate was evaporated. An oil was obtained which crystallised on standing. There was thus obtained 1-prop-2-ynylpiperazine (23 g); NMR Spectrum: (CDCl₃) 2.2 (s, 1H), 2.5 (br s, 4H), 2.85 (m, 4H), 3.25 (s, 2H).

### Example 4

### 7-(2-chloroethoxy)-4-(5-chloro-2,3-methylenedioxypyrid-4-ylamino)-5-tetrahydropyran-4-yloxyquinazoline

Using an analogous procedure to that described in Example 1, 4-chloro-7-(2-chloroethoxy)-5-tetrahydropyran-4-yloxyquinazoline was reacted with 4-amino-5-chloro-2,3-methylenedioxypyridine to give the title compound in 37% yield; NMR Spectrum: (CDCl₃) 2.0 (m, 2H), 2.3 (m, 2H), 3.65 (m, 2H), 3.9 (m, 2H), 4.1 (m, 2H), 4.4 (m, 2H), 4.8 (m, 1H), 6.2 (s, 2H), 6.65 (s, 1H), 6.9 (s, 1H), 7.8 (s, 1H), 8.6 (s, 1H), 9.5 (s, 1H); Mass Spectrum: M+H⁺ 479 and 481.

The 4-chloro-7-(2-chloroethoxy)-5-tetrahydropyran-4-yloxyquinazoline used as a starting material was prepared as follows:

Di-tert-butyl azodicarboxylate (0.338 g) was added to a stirred mixture of 4-chloro-7-hydroxy-5-tetrahydropyran-4-yloxyquinazoline (International Patent Application WO 01/94341, Example 15, Note [10] thereof; 0.25 g), 2-chloroethanol (0.073 ml), triphenylphosphine (0.385 g) and methylene chloride (15 ml) and the reaction mixture was stirred at ambient temperature for 1 hour. The mixture was concentrated to a volume of about 5 ml by evaporation and the residue was purified by column chromatography on silica using increasingly polar mixtures of petroleum ether (b.p 40-60° C.) and ethyl acetate as eluent. There was thus obtained 4-chloro-7-(2-chloroethoxy)-5-tetrahydropyran-4-yloxyquinazoline as a solid (0.17 g); NMR Spectrum: (CDCl₃) 2.0 (m, 2H), 2.15 (m, 2H), 3.7 (m, 2H), 3.95 (t, 2H), 4.1 (m, 2H), 4.4 (t, 2H), 4.8 (m, 1H), 6.7 (s, 1H), 6.95 (s, 1H), 8.85 (s, 1H).

### Example 5

### 7-(2-chloroethoxy)-4-(5-chloro-2,3-methylenedioxypyrid-4-ylamino)-5-isopropoxyquinazoline

Using an analogous procedure to that described in Example 1, 4-chloro-7-(2-chloroethoxy)-5-isopropoxyquinazoline was reacted with 4-amino-5-chloro-2,3-methylenedioxypyridine to give the title compound in 86% yield; NMR Spectrum: (CDCl₃) 1.55 (d, 6H), 3.9 (t, 2H), 4.4 (t, 2H), 4.9 (m, 1H), 6.2 (s, 2H), 6.6 (s, 1H), 6.85 (s, 1H), 7.75 (s, 1H), 8.6 (s, 1H), 9.65 (s, 1H); Mass Spectrum: M+H⁺ 437 and 439.

The 4-chloro-7-(2-chloroethoxy)-5-isopropoxyquinazoline used as a starting material was prepared as follows:

Di-tert-butyl azodicarboxylate (28.9 g) was added to a stirred mixture of 7-benzyloxy-5-hydroxy-3-pivaloyloxymethyl-3,4-dihydroquinazolin-4-one (International Patent Application WO 01/94341, Example 15, Note [8] thereof; 30 g), isopropanol (7.3 ml), triphenylphosphine (32.95 g) and methylene chloride (350 ml) that had been cooled to 0° C. The reaction mixture was allowed to warm to ambient temperature and was stirred for 1.5 hours. The mixture was evaporated and the residue was purified by column chromatography on silica using increasingly polar mixtures of methylene chloride and methanol as eluent. There was thus obtained 7-benzyloxy-5-isopropoxy-3,4-dihydroquinazolin-4-one as a solid (23.8 g); NMR Spectrum: (DMSOd₆) 7.89 (s, 1H), 7.5-7.3 (m, 5H), 6.75 (s, 1H), 6.62 (s, 1H), 5.24 (s, 2H), 4.65 (m, 1H), 1.29 (d, 6H).

Ammonium formate (48.4 g) was added to a stirred mixture of 7-benzyloxy-5-isopropoxy-3,4-dihydroquinazolin-4-one (23.8 g), 10% palladium-on-carbon catalyst (2.8 g) and DMP (300 ml) and the resultant mixture was stirred at ambient temperature for 2 hours. The mixture was filtered and the filtrate was evaporated. The material so obtained was triturated under water, the pH of which was adjusted to pH7. The solid so obtained was collected by filtration, washed with water and with diethyl ether and dried over phosphorus pentoxide under vacuum. There was thus obtained 7-hydroxy-5-isopropoxy-3,4-dihydroquinazolin-4-one as a white solid (15.9 g); NMR Spectrum: (DMSOd₆) 1.3 (d, 6H), 4.57 (m, 1H), 6.42 (s, 1H), 6.5 (s, 1H), 7.8 (s, 1H).

A mixture of the material so obtained, acetic anhydride (34 ml) and pyridine (0.62 ml) was heated to 70° C. for 30 minutes. The reaction mixture was cooled to ambient temperature and the excess of acetic anhydride was evaporated. The white solid so obtained was added to hot water (80° C., 250 ml) and the mixture was stirred vigorously and heated to 80° C. for 20 minutes. The mixture was cooled to ambient temperature and the solid was isolated and dried over phosphorus pentoxide. There was thus obtained 7-acetoxy-5-isopropoxy-3,4-dihydroquinazolin-4-one (17.86 g); NMR Spectrum: (DMSOd₆) 7.97 (s, 1H), 6.91 (s, 1H), 6.85 (s, 1H), 4.65 (m, 1H), 2.32 (s, 3H), 1.33 (d, 6M).

A mixture of a portion (5.4 g) of the material so obtained, triphenylphosphine (10.8 g), carbon tetrachloride (12 ml) and 1,2-dichloroethane (50 ml) was stirred and heated to 70° C. for 2 hours. The mixture was cooled to ambient temperature and the solvent was evaporated. The residue was dissolved in a 0.5M solution of ammonia gas in 1,4-dioxane (250 ml) and the mixture was heated to 70° C. for 10 minutes. The solvent was evaporated and the residue was cooled in an ice-water bath. Methylene chloride and water were added and the aqueous layer was brought to pH7 by the addition of dilute aqueous hydrochloric acid. The mixture was filtered. The organic phase was dried over magnesium sulphate and evaporated to give 4-chloro-7-hydroxy-5-isopropoxyquinazoline as a foam which was used without further purification.

Di-tert-butyl azodicarboxylate (7.9 g) was added to a stirred mixture of the 4-chloro-7-hydroxy-5-isopropoxyquinazoline so obtained, 2-chloroethanol (1.5 ml), triphenylphosphine (8 g) and methylene chloride (200 ml) and the reaction mixture was stirred at ambient temperature for 4 hours. The mixture was concentrated by evaporation and the residue was purified by column chromatography on silica using increasingly polar mixtures of petroleum ether (b.p 40-60° C.) and ethyl acetate as eluent. There was thus obtained 4chloro-7-(2-chloroethoxy)-5-isopropoxyquinazoline (2.5 g); NMR Spectrum: (CDCl₃) 1.45 (d, 6H), 3.9 (t, 2H), 4.4 (t, 2H), 4.75 (m, 1H), 6.65 (s, 1H), 6.9 (s, 1H), 8.8 (s, 1H).

### Example 6

Using an analogous procedure to that described in Example 3, the appropriate 7-haloalkoxyquinazoline was reacted with the appropriate heterocyclic compound to give the compounds described in Table I. Unless otherwise stated, each compound described in Table I was obtained as a free base.

TABLE I

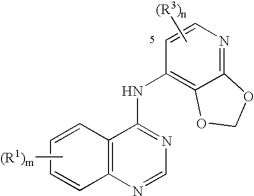

| Compound | | |
|---|---|---|
| No. & Note | (R¹)ₘ | (R³)ₙ |
| [1] | 6-methoxy-7-[3-(4-isobutyrylpiperazin-1-yl)propoxy] | 5-chloro |
| [2] | 6-methoxy- | 5-chloro |
| | 7-{3-[4-(2,2,2-trifluoroethyl)piperazin-1-yl]propoxy} | |
| [3] | 6-methoxy-7-[2-(4-prop-2-ynylpiperazin-1-yl)ethoxy] | 5-chloro |
| [4] | 5-tetrahydropyran-4-yloxy- | 5-chloro |
| | 7-[2-(4-acetylpiperazin-1-yl)ethoxy] | |
| [5] | 5-tetrahydropyran-4-yloxy- | 5-chloro |
| | 7-{2-[(3RS,4SR)-3,4-methylenedioxypyrrolidin-1-yl]ethoxy} | |
| [6] | 5-isopropoxy-7-[2-(4-acetylpiperazin-1-yl)ethoxy] | 5-chloro |
| [7] | 5-isopropoxy- | 5-chloro |
| | 7-{2-[(3RS,4SR)-3,4-methylenedioxypyrrolidin-1-yl]ethoxy} | |
| [8] | 6-(2-morpholinoethoxy)-7-methoxy | 5-chloro |
| [9] | 6-[2-(4-methylpiperazin-1-yl)ethoxy]-7-methoxy | 5-chloro |
| [10] | 6-(2-pyrrolidin-1-ylethoxy)-7-methoxy | 5-chloro |
| [11] | 6-[2-(4-acetylpiperazin-1-yl)ethoxy]-7-methoxy | 5-chloro |
| [12] | 6-{2-[(3RS,4SR)-3,4-methylenedioxypyrrolidin- | 5-chloro |
| | 1-yl]ethoxy}-7-methoxy | |
| [13] | 6-(3-pyrrolidin-1-ylpropoxy)-7-methoxy | 5-chloro |
| [14] | 6-(3-morpholinopropoxy)-7-methoxy | 5-chloro |
| [15] | 6-[3-(4-acetylpiperazin-1-yl)propoxy]-7-methoxy | 5-chloro |
| [16] | 6-[3-(4-methylpiperazin-1-yl)propoxy]-7-methoxy | 5-chloro |
| [17] | 6-{3-[(3RS,4SR)-3,4-methylenedioxypyrrolidin- | 5-chloro |
| | 1-yl]propoxy}-7-methoxy | |
| [18] | 5-tetrahydropyran-4-yloxy-7-[2-(4-prop-2-ynylpiperazin- | 5-chloro |
| | 1-yl)ethoxy] | |
| [19] | 5-tetrahydropyran-4-yloxy-7-(2-morpholinoethoxy) | 5-chloro |
| [20] | 5-tetrahydropyran-4-yloxy-7-(3-morpholinopropoxy) | 5-chloro |
| [21] | 5-tetrahydropyran-4-yloxy-7-[3-(4-prop-2-ynylpiperazin- | 5-chloro |
| | 1-yl)propoxy] | |
| [22] | 5-isopropoxy-7-(2-piperazin-1-ylethoxy) | 5-chloro |
| [23] | 5-isopropoxy-7-{2-[4-(2-hydroxyethyl)piperazin- | 5-chloro |
| | 1-yl]ethoxy} | |
| [24] | 5-isopropoxy-7-(2-pyrrolidin-1-ylethoxy) | 5-chloro |
| [25] | 5-isopropoxy-7-(2-piperidinoethoxy) | 5-chloro |
| [26] | 5-isopropoxy-7-(2-morpholinoethoxy) | 5-chloro |
| [27] | 5-isopropoxy-7-[2-(4-prop-2-ynylpiperazin-1-yl)ethoxy] | 5-chloro |
| [28] | 5-isopropoxy-6-{2-[(3RS,4SR)-3,4-dimethoxypyrrolidin- | 5-chloro |
| | 1-yl]ethoxy} | |
| [29] | 6-{2-[(3RS,4SR)-3,4-ethylidenedioxypyrrolidin- | 5-chloro |
| | 1-yl]ethoxy}-5-isopropoxy | |
| [30] | 5-isopropoxy-7-[2-(4-methylpiperazin-1-yl)ethoxy] | 5-chloro |
| [31] | 5-isopropoxy-7-(3-morpholinopropoxy) | 5-chloro |
| [32] | 7-(3-morpholinopropoxy) | 5-chloro |
| [33] | 7-[3-(4-acetylpiperazin-1-yl)propoxy] | 5-chloro |
| [34] | 6-methoxy-7-[2-(4-prop-2-ynylpiperazin-1-yl)ethoxy] | hydrogen |
| [35] | 6-methoxy-7-[3-(4-prop-2-ynylpiperazin-1-yl)propoxy] | hydrogen |

Notes
[1] The reactants were 4-(5-chloro-2,3-methylenedioxypyrid-4-ylamino)-7-(3-chloropropoxy)-6-methoxyquinazoline and 1-isobutyrylpiperazine. The reaction mixture was heated to 120° C. for 3 hours. The reaction product was purified by column chromatography on a C18 reversed phase silica column (Waters Symmetry column, 5 microns silica, 19 mm diameter, 100 mm length) using a decreasingly polar mixture of water and acetonitrile (containing 1% acetic acid) as eluent. The material so obtained was dissolved in methylene chloride and an ion exchange resin (diethylaminopolystyrene resin, 4 equivalents) was added and the mixture was stirred for 30 minutes. The mixture was filtered and the filtrate was evaporated. The resultant residue was triturated under pentane to give the required product in 51% yield which gave the following characterising data; NMR Spectrum: (CDCl₃) 1.1 (d, 6H), 2.1 (m, 2H), 2.45 (m, 4H), 2.55 (m, 2H), 2.75 (m, 1H), 3.5 (m, 2H), 3.6 (m, 2H), 4.0 (s, 3H), 4.25 (t, 2H), 6.1 (s, 2H), 7.1 (br s, 1H), 7.3 (s, 1H), 7.75 (s, 1H), 8.7 (br s, 1H); Mass Spectrum: M+H⁺ 543 and 545.

The 1-isobutyrylpiperazine used as a starting material was prepared as follows:

Isobutyryl chloride (3.25 ml) was added dropwise to a stirred mixture of 1-benzylpiperazine (5 g), triethylamine (4.35 ml) and methylene chloride (75 ml) which was cooled to 0° C. The reaction mixture was allowed to warm to ambient temperature and stirred for 1 hour. The mixture was partitioned between methylene chloride and water. The organic phase was washed with water and with brine, dried over magnesium sulphate and evaporated. The residue was purified by column chromatography on silica using a 3:2 mixture of methylene chloride and ethyl acetate as eluent. There was thus obtained 1-benzyl-4-isobutyrylpiperazine (5.95 g) as an oil; NMR Spectrum: (CDCl₃) 1.1 (d, 6H), 2.45 (m, 4H), 2.8 (m, 1H), 3.5 (m, 4H), 3.65 (m, 2H), 7.3 (m, 5H); Mass Spectrum: M+H⁺ 247.

A mixture of the material so obtained, cyclohexene (70 ml), palladium oxide-on-carbon catalyst (20%; 1.1 g) and ethanol (120 ml) was stirred and heated to 80° C. for 3 hours. The catalyst was removed by filtration and the solvent was evaporated to give 1-isobutyrylpiperazine (3.7 g) as a solid; NMR Spectrum: (CDCl₃) 1.05 (d, 61), 2.75 (m, 1H), 2.8 (m, 4H), 3.45 (m, 2H), 3.55 (m, 2H).

[2] The reactants were 4-(5-chloro-2,3-methylenedioxypyrid-4-ylamino)-7-(3-chloropropoxy)-6-methoxyquinazoline and 1-(2,2,2-trifluoroethyl)piperazine. The reaction mixture was heated to 120° C. for 3 hours. The reaction product was purified by column chromatography on a C18 reversed phase silica column (Waters Symmetry column, 5 microns silica, 19 mm diameter, 100 mm length) using a decreasingly polar mixture of water and acetonitrile (containing 1% acetic acid) as eluent. The material so obtained was dissolved in methylene chloride and an ion exchange resin (diethylaminopolystyrene resin, 4 equivalents) was added and the mixture was stirred for 30 minutes. The mixture was filtered and the filtrate was evaporated. The resultant residue was triturated under pentane to give the required product in 72% yield which gave the following characterising data; NMR Spectrum: (CDCl₃) 2.1 (m, 2H), 2.5 (m, 6H), 2.7 (m, 4H), 2.95 (q, 2H), 4.05 (s, 3H), 4.25 (t, 2H), 6.1 (s, 2H), 7.1 (br s, 1H), 7.3 (s, 1H), 7.75 (s, 1H), 8.35 (br s, 1H); Mass Spectrum: M+H⁺ 555 and 557; Elemental Analysis: Found C, 51.8; H, 5.0; N, 14.8; C₂₄H₂₆ClF₃N₆O₄ requires C, 51.9; H, 4.7; N, 15.1%.

The 1-(2,2,2-trifluoroethyl)piperazine used as a starting material was prepared as follows:

2,2,2-Trifluoroethyl trifluoromethanesulphonate (8.2 g) was added to a stirred mixture of 1-tert-butoxycarbonylpiperazine (6 g), potassium carbonate (5.77 g) and acetonitrile (30 ml) and the resultant mixture was stirred at ambient temperature for 16 hours. The mixture was filtered and the filtrate was evaporated. The residue was purified by column chromatography on silica using increasingly polar mixtures of petroleum ether (b.p 40-60° C.) and ethyl acetate as eluent. There was thus obtained tert-butyl 4-(2,2,2-trifluoroethylpiperazine-1-carboxylate as a solid (8.1 g); NMR Spectrum: (CDCl₃) 1.45 (s, 9H), 2.6 (m, 4H), 2.95 (q, 2H), 3.4 (m, 4H).

Hydrogen chloride gas was bubbled through a solution of tert-butyl 4-(2,2,2-trifluoroethylpiperazine-1-carboxylate (8 g) in ethyl acetate (50 ml) during 1.5 hours. A precipitate formed as carbon dioxide gas was evolved. The precipitate was collected by filtration, washed with ethyl acetate and dried under vacuum. There was thus obtained 1-(2,2,2-trifluoroethyl)piperazine hydrochloride (7 g); NMR Spectrum: DMSOd₆ and CF₃CO₂D) 2.85 (m, 4H), 3.1 (m, 4H), 3.35 (q, 2H).

The material so obtained was suspended in methylene chloride and a saturated methanolic ammonia solution (20 ml) was added. The resultant mixture was stirred at ambient temperature for 20 minutes. The mixture was filtered and the filtrate was evaporated at ambient temperature under vacuum. There was thus obtained 1-(2,2,2-trifluoroethyl)piperazine which was used without any additional purification.

[3] The reactants were 7-(2-chloroethoxy)-4-(5-chloro-2,3-methylenedioxypyrid-4-ylamino)-6-methoxyquinazoline and 1-prop-2-ynylpiperazine. The required product was obtained in 52% yield and gave the following characterising data; NMR Spectrum: (MSOd₆ and CF₃CO₂D) 3.3 (br s, 4H), 3.6 (br s, 4H), 3.75 (br s, 3H), 3.95 (s, 2H), 4.05 (s, 3H), 4.65 (t, 2H), 6.3 (s, 2H), 7.5 (s, 1H), 7.9 (s, 1H), 8.2 (s, 1H), 9.0 (s, 1H); Mass Spectrum: M+H⁺ 497 and 499; Elemental Analysis: Found C, 56.3; H, 5.4; N, 16.2; C₂₄H₂₅ClN₆O₄ 0.7H₂O requires C, 56.6; H, 5.2; N, 16.5%.

[4] The reactants were 7-(2-chloroethoxy)-4-(5-chloro-2,3-methylenedioxypyrid-4-ylamino)-5-tetrahydropyran-4-yloxyquinazoline and 1-acetylpiperazine. The reaction mixture was heated to 80° C. for 3 hours and then to 110° C. for 5 hours. The reaction product was purified by column chromatography on a C18 reversed phase silica column (Waters Symmetry column, 5 microns silica, 19 mm diameter, 100 mm length) using a decreasingly polar mixture of water and acetonitrile (containing 1% acetic acid) as eluent. The organic solvents were evaporated and the pH of the aqueous phase was adjusted to 7.5. The solution was extracted with methylene chloride and the organic phase was dried over magnesium sulphate and evaporated. The resultant residue was triturated under diethyl ether to give the required product in 45% yield which gave the following characterising data; NMR Spectrum: (CDCl₃) 2.0 (m, 2H), 2.1 (s, 3H), 2.3 (m, 2H), 2.6 (m, 4H), 2.95 (m, 2H), 3.55 (m, 2H), 3.65 (m, 4H), 4.1 (m, 2H), 4.3 (m, 2H), 4.8 (m, 1H), 6.2 (s, 2H), 6.6 (s, 1H), 6.9 (s, 1H), 7.8 (s, 1H), 8.65 (s, 1H), 9.5 (s, 1H); Mass Spectrum: M+H⁺ 571 and 573; Elemental Analysis: Found C, 55.3; H, 5.4; N, 13.9; C₂₇H₃₁ClN₆O₆ 1H₂O requires C, 55.1; H, 5.7; N, 14.3.

[5] The reactants were 7-(2-chloroethoxy)-4-(5-chloro-2,3-methylenedioxypyrid-4-ylamino)-5-tetrahydropyran-4-yloxyquinazoline and (3RS,4SR)-3,4-methylenedioxypyrrolidine. The reaction mixture was heated to 80° C. for 3 hours and then to 110° C. for 5 hours. The reaction product was purified by column chromatography on a C18 reversed phase silica column (Waters Symmetry column, 5 microns silica, 19 mm diameter, 100 mm length) using a decreasingly polar mixture of water and acetonitrile (containing 1% acetic acid) as eluent. The organic solvents were evaporated and the pH of the aqueous phase was adjusted to 7.5. The solution was extracted with methylene chloride and the organic phase was dried over magnesium sulphate and evaporated. The resultant residue was triturated under diethyl ether to give the required product in 69% yield which gave the following characterising data; NMR Spectrum: (CDCl₃) 2.0 (m, 2H), 2.3 (m, 2H), 2.4 (m, 2H), 2.3 (t, 2H), 3.3 (d, 2H), 3.55 (m, 2H), 4.1 (m, 2H), 4.3 (t, 2H), 4.65 (m, 2H), 4.8 (m, 1H), 4.9 (s, 1H), 5.2 (s, 1H), 6.2 (s, 2H), 6.6 (s, 1H), 6.9 (s, 1H), 7.8 (s, 1H), 8.65 (s, 1H), 9.5 (s, 1H); Mass Spectrum: M+H⁺ 558 and 560; Elemental Analysis: Found C, 56.5; H, 5.3; N, 12.5; C₂₆H₂₈ClN₅O₇ 0.2Et₂O requires C, 56.2; H, 5.3; N, 12.2%.

The (3RS,4SR)-3,4-methylenedioxypyrrolidine used as a starting material was prepared as follows:

A solution of di-tert-butyl dicarbonate (Boc₂O, 78.95 g) in ethyl acetate (125 ml) was added dropwise to a stirred mixture of 3-pyrroline (25 g; 65% pure containing pyrrolidine) and ethyl acetate (125 ml) which had been cooled to 0° C. The reaction temperature was maintained at 5-10° C. during the addition. The resultant reaction mixture was allowed to warm to ambient temperature overnight. The reaction mixture was washed successively with water, 0.1N aqueous hydrochloric acid solution, water, a saturated aqueous sodium bicarbonate solution and brine, dried over magnesium sulphate and evaporated. There was thus obtained, as a colorless oil (62 g), a 2:1 mixture of tert-butyl 3-pyrroline-1-carboxylate, NMR: (CDCl₃) 1.45 (s, 9H), 4.1 (d, 4H), 6.75 (m, 2H), and tert-butyl pyrrolidine-1-carboxylate, NMR: (CDCl₃) 1.5 (s, 9H), 1.8 (br s, 4H), 3.3 (br s, 4H).

A solution of the mixture of materials so obtained in acetone (500 ml) was added dropwise to a mixture of N-methylmorpholine-N-oxide (28.45 g), osmium tetroxide (1 g) and water (500 ml) whilst keeping the reaction temperature below 25° C. The reaction mixture was then stirred at ambient temperature for 5 hours. The solvent was evaporated and the residue was partitioned between ethyl acetate and water. The organic phase was washed with brine, dried over magnesium sulphate and evaporated. The residue was purified by column chromatography on silica using increasingly polar mixtures of petroleum ether (b.p. 40-60° C.) and ethyl acetate as eluent and by further column chromatography on silica using increasingly polar mixtures of methylene chloride and methanol. There was thus obtained tert-butyl (3RS,4SR)-3,4-dihydroxypyrrolidine-1-carboxylate as an oil (34.6 g); NMR Spectrum: (CDCl₃) 1.45 (s, 9H), 2.65 (m, 2H), 3.35 (m, 2H), 3.6 (m, 2H), 4.25 (m, 2H).

A solution of tert-butyl (3RS,4SR)-3,4-dihydroxypyrrolidine-1-carboxylate (34.6 g) in DMF (400 ml) was cooled to 0-5° C. and sodium hydride (60% dispersion in mineral oil, 0.375 mol) was added portionwise. The reaction mixture was stirred at 5° C. for 1 hour. Dibromomethane (15.6 ml) was added and the reaction mixture was stirred at 5° C. for 30 minutes. The reaction mixture was allowed to warm to ambient temperature and was stirred for 16 hours. The DMF was evaporated and the residue was partitioned between ethyl acetate and water. The organic phase was washed with water and with brine, dried over magnesium sulphate and evaporated. The residue was purified by column chromatography on silica using increasingly polar mixtures of petroleum ether (b.p. 40-60° C.) and ethyl acetate as eluent. There was thus obtained tert-butyl (3RS,4SR)-3,4-methylenedioxypyrrolidine-1-carboxylate as a colourless oil (19.77 g); NMR Spectrum: (CDCl₃) 1.45 (s, 9H), 3.35 (m, 2H), 3.75 (br s, 2H), 4.65 (m, 2H), 4.9 (s, 1H), 5.1 (s, 1H).

A cooled 5M solution of hydrogen chloride in isopropanol (150 ml) was added to a solution of tert-butyl (3RS,4SR)-3,4-methylenedioxypyrrolidine-1-carboxylate (19.7 g) in methylene chloride (500 ml) that was cooled in an ice bath. The reaction mixture was allowed to warm to ambient temperature and was stirred for 4 hours. The solvent was evaporated and the residue was triturated under diethyl ether. The precipitate was collected by filtration, washed with diethyl ether and dried. There was thus obtained (3RS,4SR)-3,4-methylenedioxypyrrolidine hydrochloride as a beige solid (13.18 g); NMR Spectrum: (DMSOd₆) 3.15 (m, 2H), 3.35 (m, 2H), 4.65 (s, 1H), 4.8 (m, 2H), 5.1 (s, 1H).

The material so obtained was suspended in diethyl ether and a saturated methanolic ammonia solution was added. The resultant mixture was stirred at ambient temperature for 10 minutes. The mixture was filtered and the solvent was evaporated at ambient temperature under vacuum. There was thus obtained (3RS,4SR)-3,4-methylenedioxypyrrolidine which was used without any additional purification.

[6] The reactants were 7-(2-chloroethoxy)-4-(5-chloro-2,3-methylenedioxypyrid-4-ylamino)-5-isopropoxyquinazoline and 1-acetylpiperazine. The reaction mixture was heated to 85° C. for 8 hours. The reaction product was purified by column chromatography on silica using increasingly polar mixtures of methylene chloride and methanol as eluent. The product was obtained in 89% yield and gave the following characterising data; m.p. 208-210° C.; NMR Spectrum: (CDCl₃) 1.55 (d, 6H), 2.1 (s, 3H), 2.6 (m, 4H), 2.9 (t, 2H), 3.5 (t, 2H), 3.7 (t, 2H), 4.25 (t, 2H), 4.85 (m, 1H), 6.15 (s, 2H), 6.55 (s, 1H), 6.85 (s, 1H), 7.75 (s, 1H), 8.6 (s, 1H), 9.6 (s, 1H); Mass Spectrum: M+H⁺ 529 and 531; Elemental Analysis: Found C, 57.0; H, 5.7; N, 15.7; C25H₂₉ClN₆O₅ requires C, 56.8; H, 5.5; N, 15.9%.

[7] The reactants were 7-(2-chloroethoxy)4-(5-chloro-2,3-methylenedioxypyrid-4-ylamino)-5-isopropoxyquinazoline and (3RS,4SR)-3,4-methylenedioxypyrrolidine. The reaction mixture was heated to 95° C. for 3 hours. The reaction product was purified by column chromatography on a C18 reversed phase silica column (Waters Symmetry column, 5 microns silica, 19 mm diameter, 100 mm length) using a decreasingly polar mixture of water and acetonitrile (containing 1% acetic acid) as eluent. The organic solvents were evaporated and the pH of the aqueous phase was adjusted to 7. The solution was extracted with methylene chloride and the organic phase was dried over magnesium sulphate and evaporated. The resultant residue was triturated under diethyl ether to give the required product in 64% yield which gave the following characterising data; NMR Spectrum: (CDCl₃) 1.55 (d, 6H), 2.35 (m, 2H), 2.9 (t, 2H), 3.25 (d, 2H), 4.25 (t, 2H), 4.6 (m, 2H), 4.85 (m, 1H), 4.9 (s, 1H), 5.15 (s, 1H), 6.15 (s, 2H), 6.55 (s, 1H), 6.85 (s, 1H), 7.75 (s, 1H), 8.6 (s, 1H), 9.6 (s, 1H); Mass Spectrum: M+H⁺ 516 and 518; Elemental Analysis: Found C, 54.7; H, 5.2; N, 13.2; C₂₄H₂₆ClN₅O₆ 0.5H₂O requires C, 54.9; H, 5.2; N, 13.3%.

[8] The reactants were 4-(5-chloro-2,3-methylenedioxypyrid-4-ylamino)-6-(2-chloroethoxy)-7-methoxyquinazoline (the preparation of which is described in Example 7 hereinafter) and morpholine. The reaction mixture was heated to 120° C. for 16 hours. The required product was obtained in 69% yield and gave the following characterising data; NMR Spectrum: (CDCl₃ and CD₃CO₂D) 3.3 (m, 4H), 3.5 (t, 2H), 3.95 (m, 4H), 4.05 (s, 3H), 4.6 (t, 2H), 6.15 (s, 2H), 7.6 (s, 1H), 7.8 (s, 2H), 8.6 (s, 1H); Mass Spectrum: M+H⁺ 460 and 462; Elemental Analysis: Found C, 53.45; H, 4.8; N, 14.5; C₂₁H₂₂ClN₅O₅ 0.55H₂O requires C, 53.7; H, 5.0; N, 14.9%.

[9] The reactants were 4-(5-chloro-2,3-methylenedioxypyrid-4-ylamino)-6-(2-chloroethoxy)-7-methoxyquinazoline and 1-methylpiperazine. The reaction mixture was heated to 120° C. for 16 hours. The reaction product was purified by column chromatography on a Waters X-Terra silica column (C18 reversed-phase, 5 microns, 19 mm diameter, 100 mm length; Waters Inc., Milford, Mass. 01757, USA) and eluted with decreasingly polar mixtures of an ammonium carbonate buffer (2 g/L in water) and acetonitrile. Appropriate fractions were collected, the organic solvent was evaporated and the resultant mixture was partitioned between ethyl acetate and a saturated aqueous sodium bicarbonate solution. The organic phase was dried over magnesium sulphate and evaporated. There was thus obtained the required product in 29% yield which gave the following characterising data; NMR Spectrum: (CDCl₃ and CD₃CO₂D) 2.7 (s, 3H), 3.25-3.35 (br m, 10H), 4.05 (s, 3H), 4.45 (t, 2H), 6.15 (s, 2H), 7.55 (s, 1H), 7.7 (s, 1H), 7.8 (s, 1H), 8.65 (s, 1H); Mass Spectrum: M+H⁺ 473 and 475; Elemental Analysis: Found C, 54.9; H, 5.3; N, 17.1; C₂₂H₂₅ClN₆O₄ 0.4H₂O requires C, 55.0; H, 5.4; N, 17.5%.

[10] The reactants were 4-(5-chloro-2,3-methylenedioxypyrid-4-ylamino)-6-(2-chloroethoxy)-7-methoxyquinazoline and pyrrolidine. The reaction mixture was heated to 120° C. for 16 hours. The required product was obtained in 41% yield and gave the following characterising data; NMR Spectrum: (CDCl₃ and CD₃COD) 2.15 (m, 4H), 3.3-3.6 (br s, 4H), 3.7 (t, 2H), 4.05 (s, 3I1), 4.65 (t, 2H), 6.15 (s, 2H), 7.65 (s, 1H), 7.8 (s, 1H), 7.9 (s, 1H), 8.65 (s, 1H); Mass Spectrum: M+H⁺ 444 and 446; Elemental Analysis: Found C, 55.0; H, 5.0; N, 14.9; C₂₁H₂₂ClN₅O₄ 0.7H₂O requires C, 55.25; H, 5.2; N, 15.3%.

[11] 4-(5-chloro-2,3-methylenedioxypyrid-4-ylamino)-6-(2-chloroethoxy)-7-methoxyquinazoline and 1-acetylpiperazine. The reaction mixture was lo heated to 120° C. for 16 hours. The required product was obtained in 51% yield and gave the following characterising data; NMR Spectrum: (CDCl₃ and CD₃CO₂D) 2.15 (s, 3H), 3.1 (m, 2H), 3.2 (m, 2H), 3.4 (t, 2H), 3.75 (m, 2H), 3.85 (m, 2H), 4.0 (s, 3H), 4.55 (t, 2H), 6.15 (s, 2H), 7.6 (s, 1H), 7.7 (s, 1H), 7.8 (s, 1H), 8.6 (s, 1H); Mass Spectrum: M+H⁺ 501 and 503.

[12] The reactants were 4-(5-chloro-2,3-methylenedioxypyrid-4-ylamino)-6-(2-chloroethoxy)-7-methoxyquinazoline and (3RS,4SR)-3,4-methylenedioxypyrrolidine. The reaction mixture was heated to 120° C. for 16 hours. The required product was obtained in 73% yield and gave the following characterising data; NMR Spectrum: (CDCl₃ and CD₃CO₂D) 2.95 (m, 2H), 3.45 (t, 2H), 3.65 (d, 2H), 4.05 (s, 3H), 4.55 (t, 2H), 4.8 (m, 3H), 5.2 (s, 1H), 6.15 (s, 2H), 7.6 (s, 1H), 7.75 (s, 1H), 7.8 (s, 1H), 8.65 (s, 1H); Mass Spectrum: M+H⁺ 488 and 490.

[13] The reactants were 4-(5-chloro-2,3-methylenedioxypyrid-4-ylamino)-6-(3-chloropropoxy)-7-methoxyquinazoline (the preparation of which is described in Example 8 hereinafter) and pyrrolidine. The reaction mixture was heated to 120° C. for 16 hours. The required product was obtained in 50% yield and gave the following characterising data; NMR Spectrum: (CDCl₃ and CD₃CO₂D) 2.1 (m, 4H), 2.4 (m, 2H), 3.0-3.8 (br s, 4H), 3.4 (t, 2H), 4.05 (s, 3H), 4.35 (t, 3H), 6.1 (s, 2H), 7.6 (s, 1H), 7.75 (s, 1H), 7.8 (s, 1H), 8.65 (s, 1H); Mass Spectrum: M+H⁺ 458 and 460; Elemental Analysis: Found C, 57.3; H, 5.4; N, 14.5; C₂₂H₂₄ClN₅O₄ 0.15H₂O requires C, 57.4; H, 5.3; N, 15.2%.

[14] The reactants were 4-(5-chloro-2,3-methylenedioxypyrid-4-ylamino)-6-(3-chloropropoxy)-7-methoxyquinazoline and morpholine. The reaction mixture was heated to 120° C. for 16 hours. The required product was obtained in 72% yield and gave the following characterising data; NMR Spectrum: (CDCl₃) 2.1 (m, 2H), 2.5 (m, 4H), 2.6 (t, 2H), 3.7 (m, 4H), 4.05 (s, 3H), 4.25 (t, 2H), 6.1 (s, 2H), 7.05 (s, 1H), 7.15 (s, 1H), 7.3 (s, 1H), 7.75 (s, 1H), 8.7 (s, 1H); Mass Spectrum: M+H⁺ 474 and 476.

[15] The reactants were 4-(5-chloro-2,3-methylenedioxypyrid-4-ylamino)-6-(3-chloropropoxy)-7-methoxyquinazoline and 1-acetylpiperazine. The reaction mixture was heated to 120° C. for 16 hours. The required product was obtained in 39% yield and gave the following characterising data; NMR Spectrum: (CDCl₃ and CD₃CO₂D) 2.15 (s, 3H), 2.35 (m, 2H), 3.15-3.3 (m, 6H), 3.8 (m, 2H), 3.9 (m, 2H), 4.0 (s, 3H), 4.3 (t, 2H), 6.15 (s, 2H), 7.6 (s, 1M), 7.65 (s, 1H), 7.8 (s, 1H), 8.65 (s, 1H); Mass Spectrum: M+H⁺ 515 and 517.

[16] The reactants were 4-(5-chloro-2,3-methylenedioxypyrid-4-ylamino)-6-(3-chloropropoxy)-7-methoxyquinazoline and 1-acetylpiperazine. The reaction mixture was heated to 120° C. for 16 hours. The required product was obtained in 27% yield and gave the following characterising data; NMR Spectrum: (CDCl₃ and CD₃CO₂D) 2.3 (m, 2H), 2.7 (s, 3H), 3.3 (t, 2H), 3.4 (m, 4H), 3.5 (m, 4H), 4.0 (s, 3H), 4.3 (t, 2H), 6.15 (s, 2H), 7.6 (s, 1H), 7.65 (s, 1H), 7.8 (s, 1H), 8.65 (s, 1H); Mass Spectrum: M+H⁺ 487 and 489.

[17] The reactants were 4-(5-chloro-2,3-methylenedioxypyrid-4-ylamino)-6-(3-chloropropoxy)-7-methoxyquinazoline and (3RS,4SR)-3,4-methylenedioxypyrrolidine. The reaction mixture was heated to 95° C. for 3 hours. The reaction product was purified by column chromatography on a C18 reversed phase silica column (Waters Symmetry column, 5 microns silica, 19 mm diameter, 100 mm length) using a decreasingly polar mixture of water and acetonitrile (containing 1% acetic acid) as eluent. The organic solvents were evaporated and the pH of the aqueous phase was adjusted to 7. The solution was extracted with methylene chloride and the organic phase was dried over magnesium sulphate and evaporated. The resultant residue was triturated under diethyl ether to give the required product in 57% yield which gave the following characterising data; NMR Spectrum: (CDCl₃ and CD₃CO₂D) 2.3 (m, 2H), 3.3 (m, 2H), 3.4 (t, 2H), 3.6 (d, 2H), 4.0 (s, 3H), 4.3 (t, 2H), 4.8 (m, 3H), 5.2 (s, 1H), 6.15 (s, 2H), 7.55 (s, 1H), 7.6 (s, 1H), 7.8 (s, 1H), 8.6 (s, 1H); Mass Spectrum: M+H⁺ 502 and 504.

[18] The reactants were 4-(5-chloro-2,3-methylenedioxypyrid-4-ylamino)-7-(2-chloroethoxy)-5-tetrahydropyran-4-yloxyquinazoline and 1-prop-2-ynylpiperazine. The reaction mixture was heated to 80° C. for 3 hours and then to 110° C. for 5 hours. The reaction product was purified by column chromatography on a Waters X-Terra silica column (C18 reversed-phase, 5 microns, 19 mm diameter, 100 mm length) and eluted with decreasingly polar mixtures of an ammonium carbonate buffer (2 g/L in water) and acetonitrile. Appropriate fractions were collected, the organic solvent was evaporated and the resultant mixture was partitioned between ethyl acetate and a saturated aqueous sodium bicarbonate solution. The organic phase was dried over magnesium sulphate and evaporated. There was thus obtained the required product in 54% yield which gave the following characterising data; NMR Spectrum: (DMSOd₆ and CD₃CO₂D) 1.85 (m, 2H), 2.15 (m, 2H), 2.5-3.0 (m, 10H), 3.15 (s, 1H), 3.3 (s, 2H), 3.55 (t, 2H), 3.9 (m, 2H), 4.3 (m, 2H), 5.05 (m, 1H), 6.2 (s, 2H), 6.9 (s, 2H), 7.8 (s, 1H), 8.5 (s, 1H); Mass Spectrum: M+H⁺ 567 and 569; Elemental Analysis: Found C, 55.9; H, 5.6; N, 14.0; C₂₈H₃₁ClN₆O₅ 2H₂O requires C, 55.8; H, 5.85; N, 13.9%.

[19] Using the detailed conditions described in Note [18] immediately above, 4-(5-chloro-2,3-methylenedioxypyrid-4-ylamino)-7-(2-chloroethoxy)-5-tetrahydropyran-4-yloxyquinazoline was reacted with morpholine to give the required product in 48% yield which gave the following characterising data; NMR Spectrum: (DMSOd₆ and CD₃CO₂D) 1.8 (m, 2H), 2.15 (m, 2H), 2.55 (m, 4H), 2.8 (m, 2H), 3.5 (m, 2H), 3.6 (m, 4H), 3.9 (m, 2H), 4.3 (t, 2H), 5.1 (m, 1H), 6.2 (s, 2H), 6.9 (m, 2H), 7.8 (s, 1H), 8.45 (s, 1H); Mass Spectrum: M+H⁺ 530 and 532; Elemental Analysis: Found C, 51.8; H, 5.8; N, 12.1; C₂₅H₂₈ClN₅O₆ 2.5H₂O requires C, 52.2; H, 5.8; N, 12.2%.

[20] The reactants were 4-(5-chloro-2,3-methylenedioxypyrid-4-ylamino)-7-(3-chloropropoxy)-5-tetrahydropyran-4-yloxyquinazoline (described in Example 9 hereinafter) and morpholine. The required product was obtained in 30% yield and gave the following characterising data; NMR Spectrum: (CDCl₃ and CF₃CO₂D) 2.05 (m, 2H), 2.35 (m, 4H), 3.15 (m, 2H), 3.45 (m, 2H), 3.75 (m, 4H), 3.9 (m, 2H), 4.2 (m, 6H), 5.0 (m, 1H), 6.3 (s, 2H), 6.85 (s, 1H), 7.0 (s, 1H), 7.9 (s, 1H), 8.7 (s, 1H); Mass Spectrum: M+H⁺ 544 and 546.

[21] The reactants were 4-(5-chloro-2,3-methylenedioxypyrid-4-ylamino)-7-(3-chloropropoxy)-5-tetrahydropyran-4-yloxyquinazoline and 1-prop-2-ynylpiperazine. The reaction product was purified by column chromatography on a C18 reversed phase silica column (Waters Symmetry column, 5 microns silica, 19 mm diameter, 100 mm length) using a decreasingly polar mixture of water and acetonitrile (containing 1% acetic acid) as eluent. The organic solvents were evaporated and the pH of the aqueous phase was adjusted to 9. The solution was extracted with methylene chloride and the organic phase was dried over magnesium sulphate and evaporated. The resultant residue was triturated under pentane to give the required product in 48% yield which gave the following characterising data; NMR Spectrum: (DMSOd₆ and CD₃CO₂D) 1.85 (m, 2H), 2.0 (m, 2H), 2.15 (m, 2H), 2.5-2.8 (br m, 10H), 3.15 (s, 1H), 3.3 (s, 2H), 3.55 (t, 2H), 3.9 (m, 2H), 4.2 (t, 2H), 5.05 (m, 1H), 6.2 (s, 2H), 6.85 (s, 1H), 6.9 (s, 1H), 7.8 (s, 1H), 8.45 (s, 1H); Mass Spectrum: M+H⁺ 581 and 583.

[22] The reactants were 7-(2-chloroethoxy)4-(5-chloro-2,3-methylenedioxypyrid-4-ylamino)-5-isopropoxyquinazoline and piperazine. The required product was obtained in 30% yield and gave the following characterising data; NMR Spectrum: (CDCl₃) 1.55 (d, 6H), 2.6 (m, 4H), 2.85 (t, 2H), 2.95 (m, 4H), 4.25 (t, 2H), 4.85 (m, 1H), 6.15 (s, 2H), 6.55 (s, 1H), 6.85 (s, 1H), 7.75 (s, 1H), 8.6 (s, 1H), 9.6 (s, 1H); Mass Spectrum: M+H⁺ 487 and 489; Elemental Analysis: Found C, 55.4; H, 5.5; N, 16.4; C₂₃H₂₇ClN₆O₄ 0.1Et₂O 0.6H₂O requires C, 55.65; H, 5.8; N, 16.6%.

[23] The reactants were 7-(2-chloroethoxy)-4-(5-chloro-2,3-methylenedioxypyrid-4-ylamino)-5-isopropoxyquinazoline and 1-(2-hydroxyethyl)piperazine. The reaction mixture was heated to 85° C. for 8 hours. The reaction product was purified by column chromatography on silica using increasingly polar mixtures of methylene chloride and methanol as eluent. The material so obtained was triturated under diethyl ether to give the required product in 67% yield which gave the following characterising data; NMR Spectrum: (CDCl₃) 1.5 (d, 6H), 2.5-2.7 (br m, 12H), 3.65 (t, 2H), 4.25 (t, 2H), 4.8 (m, 1H), 6.15 (s, 2H), 6.6 (s, 1H), 6.85 (s, 1H), 7.25 (s, 1H), 7.75 (s, 1H), 8.6 (s, 1H), 9.6 (s, 1H); Mass Spectrum: M+H⁺ 531 and 533; Elemental Analysis: Found C, 55.4; H, 6.05; N, 15.2; C₂₅H₃₁ClN₆O₅ 0.1Et₂O 0.5H₂O requires C, 55.7; H, 6.1; N, 15.35%.

[24] The reactants were 7-(2-chloroethoxy)-4-(5-chloro-2,3-methylenedioxypyrid-4-ylamino)-5-isopropoxyquinazoline and pyrrolidine. The reaction mixture was heated to 80° C. for 4 hours. The reaction product was purified by column chromatography on a C18 reversed phase silica column (Waters Symmetry column, 5 microns silica, 19 mm diameter, 100 mm length) using a decreasingly polar mixture of water and acetonitrile (containing 1% acetic acid) as eluent. The organic solvents were evaporated and the pH of the aqueous phase was adjusted to 9. The solution was extracted with methylene chloride and the organic phase was dried over magnesium sulphate and evaporated. The resultant residue was triturated under pentane to give the required product in 62% yield which gave the following characterising data; NMR Spectrum: (CDCl₃) 1.55 (d, 6H), 1.85 (m, 4H), 2.6 (m, 4H), 2.95 (t, 2H), 4.25 (t, 2H), 4.85 (m, 1H), 6.15 (s, 2H), 6.6 (s, 1H), 6.85 (s, 1H), 7.75 (s, 1H), 8.6 (s, 1H), 9.6 (s, 1H); Mass Spectrum: M+H⁺ 472 and 474; Elemental Analysis: Found C, 58.3; H, 5.4; N, 14.7; C₂₃H₂₆ClN₅O₄ requires C, 58.5; H, 5.55; N, 14.8%.

[25] Using the detailed conditions described in Note [24] immediately above, 4-(5-chloro-2,3-methylenedioxypyrid-4-ylamino)-7-(2-chloroethoxy)-5-tetrahydropyran-4-yloxyquinazoline was reacted with piperidine to give the required product in 52% yield which gave the following characterising data; NMR Spectrum: (CDCl₃) 1.45 (m, 2H), 1.55 (d, 56H), 1.65 (m, 4H), 2.5 (m, 4H), 2.85 (t, 2H), 4.25 (t, 2H), 4.85 (m, 1H), 6.15 (s, 2H), 6.6 (s, 1H), 6.85 (s, 1H), 7.75 (s, 1H), 8.6 (s, 1H), 9.6 (s, 1H); Mass Spectrum: M+H⁺ 486 and 488; Elemental Analysis: Found C, 59.3; H, 5.9; N, 14.4; C₂₄H₂₈ClN₅O₄ requires C, 59.3; H, 5.8; N, 14.4%.

[26] Using the detailed conditions described in Note [24] immediately above, 4-(5-chloro-2,3-methylenedioxypyrid-4-ylamino)-7-(2-chloroethoxy)-5-tetrahydropyran-4-yloxyquinazoline was reacted with morpholine to give the required product in 57% yield which gave the following characterising data; NMR Spectrum: (CDCl₃) 1.55 (d, 6H), 2.6 (m, 4H), 2.85 (t, 2H), 3.75 (m, 4H), 4.25 (t, 2H), 4.85 (m, 1H), 6.15 (s, 2H), 6.55 (s, 1H), 6.85 (s, 1H), 7.75 (s, 1H), 8.6 (s, 1H), 9.6 (s, 1H); Mass Spectrum: M+H⁺ 488 and 490; Elemental Analysis: Found C, 56.6; H, 5.4; N, 14.2; C₂₃H₂₆ClN₅O₅ requires C, 56.6; H, 5.4; N, 14.35%.

[27] Using the detailed conditions described in Note [24] immediately above, 4-(5-chloro-2,3-methylenedioxypyrid-4-ylamino)-7-(2-chloroethoxy)-5-tetrahydropyran-4-yloxyquinazoline was reacted with 1-prop-2-ynylpiperazine to give the required product in 41% yield which gave the following characterising data; NMR Spectrum: (CDCl₃) 1.55 (d, 6H), 2.25 (s, 1H), 2.65 (br m, 8H), 2.9 (t, 2H), 3.3 (s, 2H), 4.25 (t, 2H), 4.85 (m, 1H), 6.15 (s, 2H), 6.55 (s, 1H), 6.85 (s, 1H), 7.75 (s, 1H), 8.6 (s, 1H), 9.6 (s, 1H; Mass Spectrum: M+H⁺ 525 and 527; Elemental Analysis: Found C, 59.3; H, 5.4; N, 15.85; C₂₆H₂₉ClN₆O₄ requires C, 59.5; H, 5.6; N, 16.0%.

[28] The reactants were 7-(2-chloroethoxy)4-(5-chloro-2,3-methylenedioxypyrid-4-ylamino)-5-isopropoxyquinazoline and (3RS,4SR)-3,4-dimethoxypyrrolidine. The required product was obtained in 78% yield and gave the following characterising data; NMR Spectrum: (DMSOd₆ and CD₃CO₂D) 1.45 (d, 6H), 2.7 (m, 2H), 3.0 (m, 2H), 3.15 (m, 2H), 3.3 (s, 6H), 3.75 (m, 2H), 4.25 (t, 2H), 5.5 (m, 1H), 6.2 (s, 2H), 6.8 (s, 1H), 6.85 (s, 1H), 7.8 (s, 1H), 8.45 (s, 1H); Mass Spectrum: M+H⁺ 532 and 534; Elemental Analysis: Found C, 56.0; H, 5.6; N, 12.85; C₂₅H₃₀ClN₅O₆ 0.3H₂O requires C, 56.25; H, 5.7; N, 13.1%.

The (3RS,4SR)-3,4-dimethoxypyrrolidine used as a starting material was obtained as follows:

A solution of tert-butyl (3RS,4SR)-3,4-dihydroxypyrrolidine-1-carboxylate (1 g) in DMF (20 ml) was cooled to 0-5° C. and sodium hydride (60% dispersion in mineral oil, 0.433 g) was added portionwise. The reaction mixture was stirred at 5° C. for 1 hour. Methyl iodide (0.675 ml) was added and the reaction mixture was allowed to warm to ambient temperature and was stirred for 16 hours. The DMF was evaporated and the residue was partitioned between diethyl ether and water. The organic phase was washed with water and with brine, dried over magnesium sulphate and evaporated. The residue was purified by column chromatography on silica using increasingly polar mixtures of petroleum ether (b.p. 40-60° C.) and ethyl acetate as eluent. There was thus obtained tert-butyl (3RS,4SR)-3,4-dimethoxypyrrolidine-1-carboxylate as an oil (1.06 g); NMR Spectrum: (CDCl₃) 1.45 (s, 9H), 3.35 (m, 1H), 3.45 (s, 6H), 3.5 (m, 2H), 3.55 (m, 1H), 3.85 (m, 2H).

A cooled 5M solution of hydrogen chloride in isopropanol (3 ml) was added to a solution of tert-butyl (3RS,4SR)-3,4-dimethoxypyrrolidine-1-carboxylate (1 g) in methylene chloride (25 ml) that was cooled in an ice bath. The reaction mixture was allowed to warm to ambient temperature and was stirred for 16 hours. The solvent was evaporated. There was thus obtained (3RS,4SR)-3,4-dimethoxypyrrolidine hydrochloride as an oil (0.72 g); NMR Spectrum: (DMSOd₆) 3.1 (m, 2H), 3.25 (m, 2H), 3.35 (s, 6H), 4.0 (m, 2H), 9.3 (br s, 1H), 9.5 (br s, 1H).

The material so obtained was dissolved in methylene chloride and a 7M methanolic ammonia solution (0.2 ml) was added. The resultant mixture was stirred at ambient temperature for 5 minutes. The mixture was filtered and the solvent was evaporated at ambient temperature under vacuum. There was thus obtained (3RS,4SR)-3,4-dimethoxypyrrolidine which was used without any additional purification.

[29] Using the detailed conditions described in Note [24] immediately above except that the product was triturated under diethyl ether rather than under pentane, 4-(5-chloro-2,3-methylenedioxypyrid-4-ylamino)-7-(2-chloroethoxy)-5-tetrahydropyran-4-yloxyquinazoline was reacted with (3RS,4SR)-3,4-ethylidenedioxypyrrolidine to give the required product in 67% yield which gave the following characterising data; NMR Spectrum: (CDCl₃) 1.45 (d, 3H), 1.55 (d, 6H), 2.3 (d, 2H), 2.95 (m, 2H), 3.25 (d, 2H), 4.25 (t, 2H), 4.55 (m, 2H), 4.8 (m, 1H), 5.0 (m, 1H), 6.15 (s, 2H), 6.55 (s, 1H), 6.85 (s, 1H), 7.75 (s, 1H), 8.6 (s, 1H), 9.6 (s, 1H); Mass Spectrum: M+H⁺ 530 and 532; Elemental Analysis: Found C, 56.7; H, 5.5; N, 12.9; C₂₅H₂₈ClN₅O₆ 0.1Et₂O requires C, 56.8; H, 5.4; N, 13.0%.

The (3RS,4SR)-3,4-ethylidenedioxypyrrolidine used as a starting material was obtained as follows:

A solution of tert-butyl (3RS,4SR)-3,4-dihydroxypyrrolidine-1-carboxylate (0.5 g) in methylene chloride (15 ml) was cooled to 0-5° C. and acetaldehyde dimethylacetal (0.782 ml) and 4-toluenesulphonic acid (0.025 g) were added in turn. The reaction mixture was stirred at ambient temperature for 2 hours. The resultant mixture was evaporated and the residue was purified by column chromatography on silica using increasingly polar mixtures of petroleum ether (b.p. 40-60° C.) and ethyl acetate as eluent. There was thus obtained tert-butyl (3RS,4SR)-3,4-ethylidenedioxypyrrolidine-1-carboxylate as an oil (0.484 g); NMR Spectrum: (CDCl₃) 1.4 (d, 3H), 1.45 (s, 9H), 3.3 (m, 2H), 3.8 (m, 2H), 4.6 (m, 2H), 5.0 (q, 1H).

A cooled 5M solution of hydrogen chloride in isopropanol (4 ml) was added to a solution of tert-butyl (3RS,4SR)-3,4-ethylidenedioxypyrrolidine-1-carboxylate (0.475 g) in methylene chloride (25 ml) that was cooled in an ice bath. The reaction mixture was allowed to warm to ambient temperature and was stirred for 2 hours. The solvent was evaporated and the residue was triturated under diethyl ether. The precipitate was collected by filtration, washed with diethyl ether and dried. There was thus obtained (3RS,4SR)-3,4-ethylidenedioxypyrrolidine hydrochloride (0.28 g); NMR Spectrum: (DMSOd₆ and CD₃CO₂D) 1.35 (d, 3H), 3.1 (d, 2H), 3.4 (d, 2H), 4.75 (s, 2H), 4.9 (q, 1H).

The material so obtained was dissolved in methylene chloride and a 7M methanolic ammonia solution (0.2 ml) was added. The resultant mixture was stirred at ambient temperature for 5 minutes. The mixture was filtered and the solvent was evaporated at ambient temperature under vacuum. There was thus obtained (3RS,4SR)-3,4-ethylidenedioxypyrrolidine which was used without any additional purification.

[30] The reactants were 7-(2-chloroethoxy)-4-(5-chloro-2,3-methylenedioxypyrid-4-ylamino)quinazoline and 1-methylpiperazine. The required product was obtained in 74% yield and gave the following characterising data; NMR Spectrum: (CDCl₃ and CD₃CO₂D); Mass Spectrum: M+H⁺ 501 and 503; Elemental Analysis: Found C, 57.5; H, 6.5; N, 16.0; C₂₄H₂₉ClN₆O₄ 0.23H₂O requires C, 57.8; H, 6.1; N, 16.2%.

[31] The reactants were 7-(3-chloropropoxy)-4-(5-chloro-2,3-methylenedioxypyrid-4-ylamino)-5-isopropoxyquinazoline (the preparation of which is described in Example 12 hereinafter) and morpholine. The required product was obtained in 39% yield and gave the following characterising data; NMR Spectrum: (CDCl₃) 1.55 (d, 6H), 2.05 (m, 2H), 2.45 (m, 4H), 2.55 (t, 2H), 3.7 (m, 4H), 4.15 (t, 2H), 4.85 (m, 1H), 6.15 (s, 2H), 6.5 (s, 1H), 6.85 (s, 1H), 7.75 (s, 1H), 8.6 (s, 1H), 9.6 (s, 1H); Mass Spectrum: M+H⁺ 502 and 504; Elemental Analysis: Found C, 57.3; H, 5.65; N, 13.6; C₂₄H₂₈ClN₅O₅ requires C, 57.4; H, 5.6; N, 13.95%.

[32] The reactants were 7-(3-chloropropoxy)-4-(5-chloro-2,3-methylenedioxypyrid-4-ylamino)quinazoline (the preparation of which is described in Example 13 hereinafter) and morpholine. The required product was obtained in 45% yield and gave the following characterising data; NMR Spectrum: (DMSOd₆ and CF₃CO₂D) 2.3 (m, 2H), 3.15 (m, 2H), 3.35 (m, 2H), 3.5 (m, 2H), 3.7 (m, 2H), 4.05 (m, 2H), 4.35 (m, 2H), 6.3 (s, 2H), 7.35 (s, 1H), 7.6 (d, 1H), 7.9 (s, 1H), 8.7 (d, 1H), 9.05 (s, 1H); Mass Spectrum: M+H⁺ 444 and 446; Elemental Analysis: Found C, 57.0; H, 5.1; N, 15.7; C₂₁H₂₂ClN₅O₄ requires C, 56.8; H, 5.0; N, 15.8%.

[33] The reactants were 7-(3-chloropropoxy)4-(5-chloro-2,3-methylenedioxypyrid-4-ylamino)quinazoline and 1-acetylpiperazine. The required product was obtained in 34% yield and gave the following characterising data; NMR Spectrum: (DMSOd₆ and CF3CO₂D) 2.05 (s, 3H), 2.3 (s, 2H), 3.0 (m, 2H), 3.15 (m, 1H), 3.3-3.4 (m, 4H), 3.6 (m, 2H), 4.05 (m, 1H), 4.35 (m, 2H), 4.5 (m, 1H), 6.3 (s, 2H), 7.35 (s, 1H), 7.6 (d, 1H), 7.9 (s, 1H), 8.7 (d, 1H), 9.0 (s, 1H); Mass Spectrum: M+H⁺ 485 and 487; Elemental Analysis: Found C, 56.9; H, 5.4; N, 16.6; C₂₃H₂₅ClN₆O₄ 0.15Et₂O requires C, 57.1; H, 5.4; N, 16.9%.

[34] The reactants were 7-(2-chloroethoxy)-4-(2,3-methylenedioxypyrid-4-ylamino)quinazoline (the preparation of which is described in Example 14 hereinafter) and 1-prop-2-ynylpiperazine. After cooling of the reaction mixture and evaporation of the solvent, the residue was triturated under water and the resultant precipitate was isolated, washed with water and diethyl ether and dried. The required product was obtained in 60% yield and gave the following characterising data; NMR Spectrum: (CDCl₃) 2.26 (s, 1H), 2.8-2.6 (m, 8H), 2.97 (t, 2H), 3.3 (s, 2H); 4.03 (s, 3H), 4.33 (t, 2H), 6.14 (s, 2H), 6.98 (s, 1H), 7.12 (br s, 1H), 7.30 (s, 1H), 7.73 (d, 1H), 8.08 (d, 1H), 8.76 (s, 1H); Mass Spectrum: M+H⁺ 463.

[35] The reactants were 7-(3-chloropropoxy)-4-(2,3-methylenedioxypyrid-4-ylamino)quinazoline (the preparation of which is described in Example 15 hereinafter) and 1-prop-2-ynylpiperazine. The required product was obtained in 57% yield and gave the following characterising data; NMR Spectrum: (CDCl₃) 2.13 (m, 2H), 2.26 (s, 1H), 2.6 (m, 10H), 3.31 (s, 2H), 4.04 (s, 3H), 4.26 (t, 2H), 6.14 (s, 2H), 6.98 (s, 1H), 7.12 (br s, 1H), 7.31 (s, 1H), 7.72 (d, 1H), 8.08 (d, 1H), 8.76 (s, 1H); Mass Spectrum: M+H⁺ 477.

### Example 7

### 6-(2-chloroethoxy)-4-(5-chloro-2,3-methylenedioxypyrid-4-ylamino)-7-methoxyquinazoline

Using an analogous procedure to that described in Example 1, 4-chloro-6-(2-chloroethoxy)-7-methoxyquinazoline was reacted with 4-amino-5-chloro-2,3-methylenedioxypyridine to give the title compound in 59% yield; NMR Spectrum: (CDCl₃) 3.95 (t, 2H), 4.05 (s, 3H), 4.4 (t, 2H), 6.1 (s, 2H), 7.05 (s, 1H), 7.2 (s, 1H), 7.35 (s, 1H), 7.75 (s, 1H), 8.75 (s, 1H); Mass Spectrum: M+H⁺ 409 and 411.

The 4-chloro-6-(2-chloroethoxy)-7-methoxyquinazoline used as a starting material was prepared as follows:

A mixture of 6-acetoxy-7-methoxy-3,4-dihydroquinazolin-4-one (International Patent Application WO 96/15118, Example 39 thereof; 8 g), thionyl chloride (80 ml) and DMF (0.8 ml) was stirred and heated to 80° C. for 1.5 hours. The mixture was cooled to ambient temperature and the thionyl chloride was evaporated. The material so obtained was suspended in toluene and evaporated to dryness (twice). The resultant residue was diluted with methylene chloride (5 ml) and a 10:1 mixture (290 ml) of methanol and a saturated aqueous ammonium hydroxide solution was added. The resultant mixture was stirred and heated to 80° C. for 5 minutes. The solvent was evaporated and the solid residue was suspended in water. The basicity of the mixture was adjusted to pH7 by the addition of dilute aqueous hydrochloric acid solution. The resultant solid was collected by filtration, washed with water and dried under vacuum over phosphorus pentoxide. There was thus obtained 4-chloro-6-hydroxy-7-methoxyquinazoline (6.08 g) which was used without further purification; NMR Spectrum: (DMSOd₆) 4.05 (s, 3H), 7.4 (s, 1H), 7.45 (s, 1H), 8.8 (s, 1H).

Di-tert-butyl azodicarboxylate (1.53 ml) was added portionwise over a few minutes to a stirred mixture of 4-chloro-6-hydroxy-7-methoxyquinazoline (1 g), 2-chloroethanol (0.382 ml), triphenylphosphine (1.74 g) and methylene chloride (30 ml) and the reaction mixture was stirred at ambient temperature for 2 hours. The mixture was evaporated and the residue was purified by column chromatography on silica using increasingly polar mixtures of methylene chloride and ethyl acetate as eluent. There was thus obtained 4-chloro-6-(2-chloroethoxy)-7-methoxyquinazoline as a white solid (1.06 g); NMR Spectrum: (CDCl₃) 3.95 (t, 2H), 4.05 (s, 3H), 4.45 (t, 2H), 7.35 (s, 1H), 7.4 (s, 1H), 8.9 (s, 1H).

### Example 8

### 6-(3-chloropropoxy)-4-(5-chloro-2,3-methylenedioxypyrid-4-ylamino)-7-methoxyquinazoline

Using an analogous procedure to that described in Example 1, 4-chloro-6-(3-chloropropoxy)-7-methoxyquinazoline was reacted with 4-amino-5-chloro-2,3-methylenedioxypyridine to give the title compound in 58% yield; NMR Spectrum: (CDCl₃) 2.4 (m, 2H), 3.8 (t, 2H), 4.05 (s, 3H), 4.35 (t, 2H), 6.15 (s, 2H), 7.05 (s, 1H), 7.2 (s, 1H), 7.3 (s, 1H), 7.75 (s, 1H), 8.7 (s, 1H); Mass Spectrum: M+H⁺ 423 and 425.

The 4-chloro-6-(3-chloropropoxy)-7-methoxyquinazoline used as a starting material was prepared as follows:

Di-tert-butyl azodicarboxylate (1.84 g) was added portionwise over a few minutes to a stirred mixture of 4-chloro-6-hydroxy-7-methoxyquinazoline (1.2 g), 3-chloropropanol (0.572 ml), triphenylphosphine (2.1 g) and methylene chloride (30 ml) and the reaction mixture was stirred at ambient temperature for 3 hours. The mixture was evaporated and the residue was purified by column chromatography on silica using increasingly polar mixtures of methylene chloride and ethyl acetate as eluent. The material so obtained was triturated under diethyl ether. The resultant solid was isolated and dried under vacuum. There was thus obtained 4-chloro-6-(3-chloropropoxy)-7-methoxyquinazoline as a white solid (0.84 g); NMR Spectrum: (CDCl₃) 2.4 (m, 2H), 3.8 (t, 2H), 4.05 (s, 3H), 4.35 (t, 2H), 7.35 (s, 1H), 7.45 (s, 1H), 8.9 (s, 1H).

### Example 9

### 4-(5-chloro-2,3-methylenedioxypyrid-4-ylamino)-7-(3-chloropropoxy)-5-tetrahydropyran-4-yloxyquinazoline

Using an analogous procedure to that described in Example 1, 4-chloro-7-(3-chloropropoxy)-5-tetrahydropyran-4-yloxyquinazoline was reacted with 4-amino-5-chloro-2,3-methylenedioxypyridine to give the title compound in 78% yield; Mass Spectrum: M+H⁺ 493 and 495.

The 4-chloro-7-(3-chloropropoxy)-5-tetrahydropyran-4-yloxyquinazoline used as a starting material was prepared as follows:

Using an analogous procedure to that described in the portion of Example 4 that is concerned with the preparation of starting materials, 4-chloro-7-hydroxy-5-tetrahydropyran-4-yloxyquinazoline (2.5 g) was reacted with 3-chloropropanol. There was thus obtained the required starting material in 21% yield; NMR Spectrum: (DMSOd₆ and CF₃CO₂D) 1.7 (m, 2H), 2.0 (m, 2H), 2.25 (m, 2H), 3.55 (m, 2H), 3.8 (t, 2H), 3.9 (m, 2H), 4.3 (t, 2H), 4.95 (m, 1H), 6.8 (s, 1H), 6.9 (s, 1H), 9.2 (s, 1H).

### Example 10

### 4-(5-chloro-2,3-methylenedioxypyrid-4-ylamino)-7-(2,4-dimethoxybenzyloxy)-5-isopropoxyquinazoline

Using an analogous procedure to that described in Example 1, 4-chloro-7-(2,4-dimethoxybenzyloxy)-5-isopropoxyquinazoline was reacted with 4amino-5-chloro-2,3-methylenedioxypyridine to give the title compound in 75% yield; NMR Spectrum: (CDCl₃) 1.55 (d, 6H), 3.8 (s, 3H), 3.85 (s, 3H), 4.8 (m, 1H), 5.15 (s, 2H), 6.15 (s, 2H), 6.5 (m, 2H), 6.6 (s, 1H), 7.0 (s, 1H), 7.35 (d, 1H), 7.75 (s, 1H), 8.6 (s, 1H), 9.6 (s, 1H); Mass Spectrum: M+H⁺ 525 and 527.

The 4-chloro-7-(2,4-dimethoxybenzyloxy)-5-isopropoxyquinazoline used as a starting material was prepared as follows:

Sodium hydride (60% dispersion in mineral oil; 40 g) was added portionwise to a solution of isopropanol (30 g) in DMF (500 ml) that had been cooled to 5° C. The mixture was allowed to warm to ambient temperature and was stirred for 60 minutes. 5,7-Difluoro-3,4-dihydroquinazolin-4-one (International Patent Application WO 01/94341; 90 g) was added and the mixture was stirred at ambient temperature for 3 hours. The mixture was poured into water (1 litre) and, with vigorous stirring, glacial acetic acid was added to acidify the mixture to pH5. The resultant solid was isolated, washed with water and with diethyl ether and dried under vacuum. There was thus obtained 7-fluoro-5-isopropoxy-3,4-dihydroquinazolin-4-one (79 g); NMR Spectrum: (DMSOd₆) 1.31 (s, 6H), 4.73 (m, 1H), 6.89 (m, 1H), 6.95 (m, 1H), 7.96 (s, 1H); Mass Spectrum: M+H⁺ 223.

A mixture of 7-fluoro-5-isopropoxy-3,4-dihydroquinazolin-4-one (61 g), 2,4-dimethoxybenzyl alcohol (138 g), potassium tert-butoxide (185 g) and THF (1.5 litres) was stirred and heated to reflux for 18 hours. After cooling, the solvent was evaporated and a mixture of methylene chloride (400 ml) and water (600 ml) was added. With cooling, the 2-phase mixture was neutralised by the addition of 2N aqueous hydrochloric acid. The mixture was filtered and the organic phase was separated, dried over magnesium sulphate and evaporated. The residue was triturated under diethyl ether. There was thus obtained 7-(2,4-dimethoxybenzyloxy)-5-isopropoxy-3,4-dihydroquinazolin-4-one (68 g); NMR Spectrum: (DMSOd₆) 1.28 (s, 6H), 3.78 (s, 3H), 3.82 (s, 3H), 4.63 (m, 1H), 5.06 (s, 2H), 6.55 (m, 2H), 6.62 (s, 1H), 6.71 (s, 1H), 7.33 (d, 1H), 7.88 (s, 1H); Mass Spectrum: M+H⁺ 371.

A mixture of a portion (4 g) of the material so obtained, phosphorus oxychloride (1.98 g), diisopropylethylamine (3.6 g) and methylene chloride (100 ml) was stirred and heated 5 to 75° C. for 3 hours. The mixture was cooled and evaporated. The residue was dried under vacuum for 1 hour and purified by column chromatography on silica using a 20:3 mixture of methylene chloride and ethyl acetate as eluent. There was thus obtained 4-chloro-7-(2,4-dimethoxybenzyloxy)-5 isopropoxyquinazoline as a solid (2.63 g); NMR Spectrum: (CDCl₃) 1.46 (s, 3H), 1.47 (s, 3H), 3.83 (s, 3H), 3.85 (s, 3H), 4.68 (m, 1H), 5.16 (s, 2H), 6.52 (m, 2H), 6.65 (s, 1H), 7.06 (s, 1H), 7.33 (d, 1H), 8.78 (s, 1H); Mass Spectrum: M+H⁺ 389.

### Example 11

### 4-(5-chloro-2,3-methylenedioxypyrid-4-ylamino)-7-hydroxy-5-isopropoxyquinazoline

Trifluoroacetic acid (4.5 ml) was added to a solution of 4-(5-chloro-2,3-methylenedioxypyrid-4-ylamino)-7-(2,4-dimethoxybenzyloxy)-5-isopropoxyquinazoline (0.53 g) in methylene chloride (9 ml) and the reaction mixture was stirred at ambient temperature for 30 minutes. The solvents were evaporated to give the di-trifluoroacetic acid salt (0.618 g) of the required compound. A portion of this salt was dissolved in methylene chloride (2 ml) and a 7M methanolic ammonia solution was added. The mixture was filtered and the filtrate was evaporated. There was thus obtained the title compound; Mass Spectrum: M+H⁺ 375 and 377.

### Example 12

### 4-(5-chloro-2,3-methylenedioxypyrid-4-ylamino)-7-(3-chloropropoxy)-5-isopropoxyquinazoline

A mixture of 4-(5-chloro-2,3-methylenedioxypyrid-4-ylamino)-7-hydroxy-5-isopropoxyquinazoline di-trifluoroacetic acid salt (0.615 g), 1,3-dichloropropane (0.38 ml), potassium carbonate (0.56 g) and DMF (6 ml) was stirred and heated to 80° C. for 5 hours. After cooling, the solids were filtered off and the filtrate was evaporated. The residue was purified by column chromatography on silica using a 24:1 mixture of methylene chloride and methanol as eluent. There was thus obtained the title compound (0.32 g); NMR Spectrum: (CDCl₃) 1.55 (d, 6H), 2.3 (m, 2H), 3.8 (t, 2H), 4.25 (t, 2H), 4.9 (m, 1H), 6.15 (s, 2H), 6.5 (s, 1H), 6.9 (s, 1H), 7.75 (s, 1H), 8.6 (s, 1H), 9.6 (s, 1H).

### Example 13

### 4-(5-chloro-2,3-methylenedioxypyrid-4-ylamino)-7-(3-chloropropoxy)quinazoline

Using an analogous procedure to that described in Example 1, 4-chloro-7-(3-chloropropoxy)quinazoline was reacted with 4-amino-5-chloro-2,3-methylenedioxypyridine to give the title compound in 89% yield; NMR Spectrum: DMSOd₆ and CF₃CO₂D) 2.25 (m, 2H), 3.8 (t, 2H), 4.35 (t, 2H), 6.25 (s, 2H), 7.35 (s, 1H), 7.6 (d, 1H), 7.9 (s, 1H), 8.7 (d, 1H), 9.0 (s, 1H).

The 4-chloro-7-(3-chloropropoxy)quinazoline used as a starting material was prepared as follows:

Sodium hydride (60% dispersion in mineral oil; 2.92 g) was added portionwise over 45 minutes to a stirred mixture of 1,3-propanediol (5.3 ml) and DMF (20 ml) that had been cooled to 0° C. The resultant mixture was stirred at ambient temperature for 1 hour and then heated to 60° C. 7-Fluoro-3,4-dihydroquinazolin-4-one (International Patent Application WO 01/04102, Example 2, Note [12] thereof; 2 g) was added and the reaction mixture was stirred and heated to 115° C. for 3.5 hours. The reaction mixture was cooled to 0° C. and water (50 ml) was added. The mixture was acidified to pH5.9 with 2N aqueous hydrochloric acid. The resultant precipitate was collected by filtration, washed with water and dried under vacuum over phosphorus pentoxide at 40° C. The solid so obtained was washed with diethyl ether and dried again under vacuum. There was thus obtained 7-(3-hydroxypropoxy)-3,4-dihydroquinazolin-4-one (2.1 g); NMR Spectrum: (DMSOd₆) 1.9 (m, 2H), 3.6 (m, 2H), 4.15 (m, 2H), 4.6 (br s, 2H), 7.1 (m, 2H), 8.05 (m, 2H); Mass Spectrum: M+H⁺ 221.

A mixture of 7-(3-hydroxypropoxy)-3,4-dihydroquinazolinone (1 g), 1,2-dichloroethane (50 ml), triphenylphosphine (5.24 g) and carbon tetrachloride (2.9 ml) was stirred and heated to 70° C. for 2 hours. The solvent was evaporated and the residue was purified by column chromatography on silica using initially methylene chloride followed by gradually increasing the polarity of the solvent up to a 9:1 mixture of methylene chloride and methanol as eluent. There was thus obtained 4-chloro-7-(3-chloropropoxy)quinazoline (1.23 g; containing 0.6 mole of triphenylphosphine oxide per mole of product); Mass Spectrum: M+H⁺ 393 and 395.

### Example 14

### 7-(2-chloroethoxy)-4-(2,3-methylenedioxypyrid-4-ylamino)-6-methoxyquinazoline

Sodium hexamethyldisilazane (1M solution in THF; 2 ml) was added dropwise to a mixture of 4-amino-2,3-methylenedioxypyridine (0.138 g), 4-chloro-7-(2-chloroethoxy)-6-methoxyquinazoline (0.272 g) and THF (5 ml) that had been cooled to 0° C. The mixture was stirred at 0° C. for 1 hour. The resultant mixture was allowed to warm to ambient temperature and was stirred for 2 hours. The reaction was quenched by the addition of glacial acetic acid (0.12 ml). The solvents were evaporated and the residue was partitioned between methylene chloride and an aqueous ammonium hydroxide solution. The organic layer was collected and concentrated to a small volume. Diethyl ether was added and a precipitate formed. The resultant solid was isolated, washed with diethyl ether and dried. There was thus obtained the title compound (0.245 g); NMR Spectrum: (DMSOd₆) 3.97 (s, 3H), 4.04 (m, 2H), 4.45 (m, 2H), 6.12 (s, 2H), 7.13 (brd, 1H), 7.25 (s, 1H), 7.60 (d, 1H), 7.83 (s, 1H), 8.47 (s, 1H), 9.87 (br s, 1H); Mass Spectrum: M+H⁺ 375.

The 4-amino-2,3-methylenedioxypyridine used as a starting material was prepared as follows:

Dibromomethane (31.5 ml) was added to a mixture 2,3-dihydroxypyridine (33 g), potassium carbonate (62 g) and NMP (200 ml) and the mixture was stirred and heated to 90° C. for 16 hours. The mixture was cooled to ambient temperature and filtered. The filtrate was partitioned between diethyl ether (5×100 ml) and water (200 ml). The organic extracts were combined and concentrated under vacuum to a volume of about 20 ml. Petroleum ether (b.p 40-60° C.; 300 ml) was added and the solution was washed with brine. The organic layer was separated and evaporated. There was thus obtained 2,3-methylenedioxypyridine as a liquid (5.1 g); NMR Spectrum: (CDCl₃) 6.05 (s, 2H), 6.76 (m, 1H), 6.99 (d, 1H), 7.65 (d, 1H).

Using an analogous procedure to that described in the second paragraph of the portion of Example 1 that is concerned with the preparation of the starting material 4-amino-5-chloro-2,3-methylenedioxypyridine, 2,3-methylenedioxypyridine was reacted with carbon dioxide gas to give 2,3-methylenedioxypyridinecarboxylic acid in 80% yield; NMR Spectrum: (DMSOd₆) 6.24 (s, 2H), 7.13 (d, 1H); 7.63 (d, 1H).

Using an analogous procedure to that described in the third paragraph of that portion of Example 1 that is concerned with the preparation of starting materials, 2,3-methylenedioxypyridine-4-carboxylic acid was reacted with diphenylphosphoryl azide and anhydrous tert-butanol to give tert-butyl 2,3-methylenedioxypyrid-4-ylcarbamate in 62% yield; Mass Spectrum: M+H⁺ 239.

Using an analogous procedure to that described in the last paragraph of that portion of Example 1 that is concerned with the preparation of starting materials, tert-butyl 2,3-methylenedioxypyrid-4-ylcarbamate was reacted with trifluoroacetic acid to give 4-amino-2,3-methylenedioxypyridine in 80% yield; NMR Spectrum: (CDCl₃) 3.98 (m, 2H), 5.98 (s, 2H), 6.24 (d, 1H), 7.44 (d, 1H); Mass Spectrum: M+H⁺ 139.

### Example 15

### 7-(3-chloropropoxy)-4-(2,3-methylenedioxypyrid-4-ylamino)-6-methoxyquinazoline

Using an analogous procedure to that described in Example 14, 4-chloro-7-(3-chloropropoxy)-6-methoxyquinazoline was reacted with 4-amino-2,3-methylenedioxypyridine to give the title compound in 68% yield; NMR Spectrum: (DMSOd₆) 2.26 (m, 2H), 3.83 (m, 2H), 3.96 (s, 3H), 4.28 (m, 2H), 6.12 (s, 2H), 7.15 (br d, 1H), 7.25 (s, 1H), 7.61 (d, 1H), 7.81 (s, 1H), 8.49 (s, 1H), 9.79 (br s, 1H); Mass Spectrum: M+H⁺ 389.

### Example 16

### 7-[2-(4-acetylpiperazin-1-yl)ethoxy]-4-(2,3-methylenedioxypyrid-4-ylamino)-5-tetrahydropyran-4-yloxyquinazoline

Using an analogous procedure to that described in Example 1, 7-[2-(4-acetylpiperazin-1-yl)ethoxy]-4-chloro-5-tetrahydropyran-4-yloxyquinazoline (0.113 g) was reacted with 4-amino-2,3-methylenedioxypyridine (0.036 g). The reaction mixture was quenched with glacial acetic acid (0.031 g) and diluted with methanol. The mixture was evaporated and the residue was purified by column chromatography on a C18 reversed phase silica column (Waters Symmetry column, 5 microns silica, 20 mm diameter, 100 mm length) using a decreasingly polar mixture of water and acetonitrile (containing 1% acetic acid) as eluent. The material so obtained was diluted with a 7M methanolic ammonia solution. The mixture was evaporated and the material so obtained was dissolved in methylene chloride. The solution was dried over magnesium sulphate and evaporated to give the title compound as a foam in 53% yield; NMR Spectrum: (CDCl₃) 2.02 (m, 2H), 2.1 (s, 3H), 2.22 (m, 2H), 2.6 (m, 4H), 2.9 (m, 2H), 3.51 (m, 2H), 3.6 (m, 2H), 3.66 (m, 2H), 4.1 (m, 2H), 4.25 (m, 2H), 4.73 (m, 1H), 6.13 (s, 2H), 6.59 (s, 1H), 6.9 (s, 1H), 7.7 (d, 1H), 8.36 (d, 1H), 8.66 (s, 1H); Mass Spectrum: M+H⁺ 537.

The 7-[2-(4-acetylpiperazin-1-yl)ethoxy]4-chloro-5-tetrahydropyran-4-yloxyquinazoline used as a starting material was prepared as follows:

Sodium hydride (60% dispersion in mineral oil; 0.6 g) was added portionwise to a solution of 4-hydroxytetrahydropyran (0.78 g) in DMF (10 ml) that had been cooled to 5° C. The mixture was allowed to warm to ambient temperature and was stirred for 15 minutes. 5,7-Difluoro-3,4-dihydroquinazolin-4-one (International Patent Application WO 01/94341; 0.9 g) was added and the mixture was stirred at ambient temperature for 30 minutes. The mixture was poured into water (100 ml) and, with vigorous stirring, glacial acetic acid was added to acidify the mixture to pH5. The resultant solid was isolated, washed with water and with diethyl ether and dried under vacuum. There was thus obtained 7-fluoro-5-tetrahydropyran-4-yloxy-3,4-dihydroquinazolin-4-one (1.1 g); NMR Spectrum: (DMSOd₆) 1.6-1.75 (m, 2H), 1.9-2.0 (m, 2H), 3.5-3.6 (m, 2H), 3.85-3.95 (m, 2H), 4.8 (m, 1H), 6.9 (m, 1H), 7.05 (m, 1H), 8.0 (s, 1H); Mass Spectrum: M+H 265.

After repetition of the prior reaction, a mixture of 7-fluoro-5-tetrahydropyran-4-yloxy-3,4-dihydroquinazolin-4-one (5.3 g), 2-piperazin-1-ylethanol (3.9 g), potassium tert-butoxide (6.7 g) and TBH (200 ml) was stirred and heated to reflux for 3 hours. A second portion (6.7 g) of potassium tert-butoxide was added and the mixture was heated to reflux for a further 12 hours. The mixture was cooled to ambient temperature and filtered. The filtrate was evaporated and the residue was purified by column chromatography on silica using increasingly polar mixtures of methylene chloride and a 7M methanolic ammonia solution as eluent. The material so obtained was triturated under diethyl ether. There was thus obtained 7-(2-piperazin-1-ylethoxy)-5-tetrahydropyran-4-yloxy-3,4-dihydroquinazolin-4-one (5.2 g); NMR Spectrum: (DMSOd₆ and CF₃CO₂D) 1.75 (m, 2H), 2.03 (m, 2M), 3.2-4.0 (m, 14H), 4.59 (m, 2H), 4.92 (m, 1H), 6.88 (s, 1H), 6.9 (s, 1H), 9.28 (s, 1H); Mass Spectrum: M+H⁺ 375.

Acetic anhydride (1.51 ml) was added dropwise to a stirred mixture of 7-(2-piperazin-1-ylethoxy)-5-tetrahydropyran-4-yloxy-3,4-dihydroquinazolin-4-one (5 g) and water (20 ml) and the resultant mixture was stirred at ambient temperature for 10 minutes. The reaction mixture was evaporated and the residue was triturated under diethyl ether. The resultant solid was isolated, washed with diethyl ether and dried under vacuum. There was thus obtained 7-[2-(4-acetylpiperazin-1-yl)ethoxy]-5-tetrahydropyran-4-yloxy-3,4-dihydroquinazolin-4one (5.5 g); NMR Spectrum: (DMSOd₆ and CF₃CO₂D) 1.75 (m, 2H), 2.03 (m, 2H), 2.08 (s, 3H), 3.0-4.2 (m, 13H), 4.56 (m, 3H), 4.94 (m, 1H), 6.84 (s, 1H), 6.9 (s, 1H), 9.21 (s, 1H); Mass Spectrum: M+H⁺ 417.

A mixture of a portion (0.416 g) of the material so obtained, triphenylphosphine (0.655 g), carbon tetrachloride (0.34 ml) and 1,2-dichloroethane (20 ml) was stirred and heated to 70° C. for 1.5 hours. The mixture was evaporated and the residue was purified by column chromatography on silica using increasingly polar mixtures of methylene chloride and a 7M methanolic ammonia solution (a solvent gradient having from 1% to 3% methanolic ammonia solution) as eluent. There was thus obtained 7-[2-(4-acetylpiperazin-1-yl)ethoxy]-4-chloro-5-tetrahydropyran-4-yloxyquinazoline as a solid (0.35 g); NMR Spectrum: (CDCl₃) 2.0 (m, 2H), 2.1 (s, 3H), 2.12 (m, 2H), 2.58 (m, 4H), 2.9 (m, 2H), 3.51 (m, 2H), 3.68 (m, 4H), 4.05 (m, 2H), 4.25 (m, 2H), 4.75 (m, 1H), 6.62 (s, 1H), 6.94 (s, 1H), 8.82 (s, 1H); Mass Spectrum: M+H⁺ 435 and 437.

### Example 17

### 7-[2-(4-acetylpiperazin-1-yl)ethoxy-4-(2,3-methylenedioxypyrid-4-ylamino)-5-isopropoxyquinazoline

Using an analogous procedure to that described in Example 16, 7-[2-(4-acetylpiperazin-1-yl)ethoxy]4-chloro-5-isopropoxyquinazoline was reacted with 4-amino-2,3-methylenedioxypyridine to give the title compound in 55% yield; NMR Spectrum: (CDCl₃) 1.55 (s, 3H), 1.56 (s, 3H), 2.1 (s, 3H), 2.59 (m, 4H), 2.89 (m, 2H), 3.51 (m, 2H), 3.67 (m, 2H), 4.24 (m, 2H), 4.85 (m, 1H), 6.13 (s, 2H), 6.57 (s, 1H), 6.85 (s, 1H), 7.71 (d, 1H), 8.41 (d, 1H), 8.66 (s, 1H); Mass Spectrum: M+H⁺ 495.

The 7-[2-(4-acetylpiperazin-1-yl)ethoxy]4-chloro-5-isopropoxyquinazoline that is required as a starting material was prepared as follows using analogous procedures to those described in the portion of Example 16 that is concerned with the preparation of starting materials.

5,7-Difluoro-3,4dihydroquinazolin-4-one was reacted with isopropanol to give 7-fluoro-5-isopropoxy-3,4-dihydroquinazolin-4-one in 73% yield; NMR Spectrum: (MSOd₆) 1.31 (s, 6H), 4.73 (m, 1H), 6.89 (m, 1H), 6.95 (m, 1H), 7.96 (s, 1H); Mass Spectrum: M+H⁺ 223.

The material so obtained was reacted with 2-piperazin-1-ylethanol to give 5-isopropoxy-7-(2-piperazin-1-ylethoxy)-3,4-dihydroquinazolin-4-one in 63% yield; NMR Spectrum: (CDCl₃) 1.45 (s, 3H), 1.46 (s, 3H), 2.4-3.0 (m, 10H), 4.2 (t, 2H), 4.62 (m, 1H), 6.51 (s, 1H), 6.72 (s, 1H), 7.9 (s, 1H).

The material so obtained was reacted with an excess of acetic anhydride but using methylene chloride rather than water as the reaction solvent. The reaction mixture was stirred at ambient temperature for 15 minutes. The mixture was partitioned between methylene chloride and a saturated aqueous sodium bicarbonate solution. The organic layer was washed with water and with brine, dried over magnesium sulphate and evaporated. The residue was triturated under a mixture of acetonitrile and diethyl ether. There was thus obtained 7-[2-(4-acetylpiperazin-1-yl)ethoxy]-5-isopropoxy-3,4-dihydroquinazolin-4-one in 70% yield; NMR Spectrum: (CDCl₃) 1.46 (s, 3H), 1.47 (s, 3H), 2.1 (s, 3H), 2.58 (m, 4H), 2.87 (t, 2H), 3.5 (m, 2H), 3.66 (m, 2H), 4.21 (t, 2H), 4.63 (m, 1H), 6.51 (s, 1H), 6.72 (s, 1H), 7.9 (s, 1H), 9.9 (br s, 1H); Mass Spectrum: M+H⁺ 375.

The material so obtained was reacted with carbon tetrachloride and triphenylphosphine to give 7-[2-(4-acetylpiperazin-1-yl)ethoxy]-4-chloro-5-isopropoxyquinazoline in 68% yield which was used without further purification.

### Example 18

### 4-(5-chloro-2,3-methylenedioxypyrid-4-ylamino)-7-{2-[4-(2-dimethylaminoacetyl)piperazin-1-yl]ethoxy}-5-isopropoxyquinazoline

4-(5-Chloro-2,3-methylenedioxypyrid-4-ylamino)-5-isopropoxy-7-(2-piperazin-1-ylethoxy)quinazoline (0.2 g) was added to a stirred mixture of 2-dimethylaminoacetyl chloride hydrochloride (0.097 g), triethylamine (0.15 ml) and methylene chloride (5 ml) that had been cooled to 0° C. The reaction mixture was allowed to warm to ambient temperature and stirred for 2 hours. A second portion of each of 2-dimethylaminoacetyl chloride hydrochloride (0.097 g) and triethylamine (0.057 ml) were added and the reaction was stirred at ambient temperature for 16 hours overnight. Methylene chloride (50 ml) was added and the reaction mixture was extracted twice with a saturated aqueous sodium bicarbonate solution. The organic phase was dried over magnesium sulphate and evaporated. The residue was purified by column chromatography on silica using increasingly polar solvent mixtures, starting with a 9:1 mixture of methylene chloride and methanol and ending with a 90:8:2 mixture of methylene chloride, methanol and a saturated methanolic ammonia solution. There was thus obtained the title compound as a foam (0.155 g); NMR Spectrum: (CDCl₃) 1.55 (d, 6H), 2.3 (s, 6H), 2.6 (m, 4H), 2.9 (t, 2H), 3.1 (s, 2H), 3.65 (m, 4H), 4.25 (t, 2H), 4.85 (s, 1H), 6.15 (s, 2H), 6.55 (s, 1H), 6.85 (s, 1H), 7.75 (s, 1H), 8.6 (s, 1H), 9.6 (s, 1H); Mass Spectrum: M+H⁺ 572 and 574; Elemental Analysis: Found C, 55.1; H, 6.1; N, 16.8; C₂₇H₃₄ClN₇O₅ 0.75H₂O requires C, 55.4; H, 6.1; N, 16.7%.

### Example 19

### 7(N-tert-butoxycarbonylpiperidin-4-ylmethoxy)-4-(5-chloro-2,3-methylenedioxypyrid-4-ylamino)-6-methoxyquinazoline

Using a similar procedure to that described in Example 1, a solution of 4-amino-5-chloro-2,3-methylenedioxypyridine (0.193 g) in DMF (2 ml) was added to a stirred suspension of sodium hydride (60% dispersion in mineral oil, 0.048 g) in DMF (2 ml) and the mixture was stirred at ambient temperature for 15 minutes. A solution of 7-(N-tert-butoxycarbonylpiperidin-4-ylmethoxy)-4-chloro-6-methoxyquinazoline [International Patent Application WO 02/16352 (Note [24] within Example 2 thereof; 0.38 g] in DMF (4 ml) was added and the resultant mixture was stirred at ambient temperature for 1 hour. The reaction mixture was partitioned between ethyl acetate and brine. The organic phase was dried over magnesium sulphate and evaporated. The residue was purified by column chromatography on silica using a 49:1 mixture of methylene chloride and methanol. There was thus obtained the title compound as a solid (0.24 g); NMR Spectrum: (DMSOd₆) 1.29 (m, 2H), 1.45 (s, 9H), 1.8 (m, 2H), 2.04 (m, 1H), 2.83 (m, 2H), 4.0 (m, 7H), 8.12 (br s, 2H), 7.17 (br s, 1H), 7.72 (m, 2H), 8.37 (br s, 1H), 9.37 (br s, 1H); Mass Spectrum: M+H⁺ 544 and 546.

### Example 20

### 4-(5-chloro-2,3-methylenedioxypyrid-4-ylamino)-6-methoxy-7-(piperidin-4-ylmethoxy)quinazoline

Trifluoroacetic acid (1 ml) was added to a solution of 7-(N-tert-butoxycarbonylpiperidin-4-ylmethoxy)-4-(5-chloro-2,3-methylenedioxypyrid-4-ylamino)-6-methoxyquinazoline (0.253 g) in methylene chloride (10 ml) and the reaction mixture was stirred at ambient temperature for 1 hour. The reaction mixture was evaporated. Toluene was added to the residue and the mixture was evaporated. The residue was purified by column chromatography on silica (Isolute SCX column) using a 7M methanolic ammonia solution as eluent. There was thus obtained the title compound as a solid (0.187 g); NMR Spectrum: (DMSOd₆) 1.25 (m, 2H), 1.75 (d, 2H), 1.93 (m, 1H), 2.54 (m, 2H), 3.0 (d, 2H), 3.93 (s, 3H), 3.98 (d, 2H), 6.17 (s, 2H), 7.15 (s, 1H), 7.76 (s, 1H), 7.78 (s, 1H), 8.23 (s, 1H); Mass Spectrum: M+H⁺ 444 and 446.

### Example 21

### 4-(5-chloro-2,3-methylenedioxypyrid-4-ylamino)-7-[N-(2-dimethylaminoacetyl)piperldin-4-ylmethoxy]-6-methoxyquinazoline

Diisopropylethylamine (0.118 ml) was added to a mixture of 4-(5-chloro-2,3-methylenedioxypyrid-4-ylamino)-6-methoxy-7-(piperidin-4-ylmethoxy)quinazoline (0.15 g), N,N-dimethylglycine (0.042 g), 2-(7-azabenzotriazol-1-yl)-1,1,3,3-tetramethyluronium hexafluorophosphate(V) (0.154 g) and DMF (3 ml) and the reaction mixture was stirred at ambient temperature for 16 hours. The mixture was diluted with ethyl acetate and washed with brine. The organic solution was dried over magnesium sulphate and evaporated. The residue was purified by column chromatography on silica using a 100:3 mixture of methylene chloride and a 7M methanolic ammonia solution as eluent. There was thus obtained the title compound as a solid (0.051 g); NMR Spectrum: (DMSOd₆) 1.11-1.36 (m, 2H), 1.83 (d, 2H), 2.11 (m, 1H), 2.19 (s, 6H), 2.61 (t, 1H), 3.03 (m, 2H), 3.12 (d, 1H), 3.93 (s, 3H), 4.06 (m, 3H), 4.4 (d, 1H), 6.19 (br s, 2H), 7.19 (br s, 1H), 7.78 (m, 2H), 8.39 (br s, 1H), 9.71 (br s, 1H); Mass Spectrum: M+H⁺ 529 and 531.

### Example 22

### 7-[2-(4-acetylpiperazin-1-yl)ethoxy]-4-(5-chloro-2,3-methylenedioxypyrid-4-ylamino)-5-isopropoxyquinazoline

A mixture of 7-(2-chloroethoxy)-4-(5-chloro-2,3-methylenedioxypyrid-4-ylamino)-5-isopropoxyquinazoline (24 g), 1-acetylpiperazine (21 g), potassium iodide (18 g) and DMA (500 ml) was stirred and heated to 100° C. for 4 hours. The solvent was evaporated and the residue was partitioned between methylene chloride (1 litre) and water (500 ml). The aqueous layer was extracted with methylene chloride. The organic solutions were combined, washed with brine, dried over magnesium sulphate and evaporated. The residue was purified by column chromatography on silica using increasingly polar mixtures of methylene chloride and methanol (from a 20:1 mixture to a 10:1 mixture) as eluent. After evaporation of the solvent, the material so obtained was triturated under diethyl ether. There was thus obtained the title compound as a white solid (26.2 g); m.p. 208-210° C.

The 7-(2-chloroethoxy)-4-(5-chloro-2,3-methylenedioxypyrid-4-ylamino)-5-isopropoxyquinazoline used as a starting material was obtained as follows:

Sodium hexamethyldisilazane (1M solution in THF, 164 ml) was added dropwise over one hour to a ice-cooled mixture of 4-chloro-7-(2,4-dimethoxybenzyloxy)-5-isopropoxyquinazoline (32 g), 4-amino-5-chloro-2,3-methylenedioxypyridine (15.6 g) and THF (430 ml) whilst maintaining the temperature of the reaction mixture at about 3° C. At the end of the addition, the reaction mixture was allowed to warm to ambient temperature and was stirred for 2.5 hours. The reaction mixture was cooled to 0° C. and a mixture of acetic acid (9.4 ml) and water (250 ml) was added. The mixture was evaporated and the residue was partitioned between methylene chloride and water, the basicity of the aqueous phase having been adjusted to 7.5 by the addition of 3N aqueous hydrochloric acid solution. The organic phase was separated and the aqueous phase was extracted three times with methylene chloride. The organic layers were combined, washed with brine, dried over magnesium sulphate and evaporated. The resultant solid was triturated under ethyl acetate. There was thus obtained 4-(5-chloro-2,3-methylenedioxypyrid-4-ylamino)-7-(2,4-dimethoxybenzyloxy)-5-isopropoxyquinazoline as a white solid (38 g); Mass Spectrum: M+H⁺ 525 and 527.

Triethylsilane (70 ml) and trifluoroacetic acid (48 ml) were added in turn to an ice-cooled solution of 4-(5-chloro-2,3-methylenedioxypyrid-4-ylamino)-7-(2,4-dimethoxybenzyloxy)-5-isopropoxyquinazoline (37.7 g) in methylene chloride (560 ml) and the resultant reaction mixture was stirred at ambient temperature for 1 hour. The solvents were evaporated under high vacuum. The resultant solid was triturated under ethyl acetate. The material so obtained was isolated, washed with ethyl acetate and dried under high vacuum. There was thus obtained the di-trifluoroacetic acid salt (37.4 g) of 4-(5-chloro-2,3-methylenedioxypyrid-4-ylamino)-7-hydroxy-5-isopropoxyquinazoline which was used without further purification.

Potassium carbonate (34.6 g) was added to a mixture of 4-(5-chloro-2,3-methylenedioxypyrid-4-ylamino)-7-hydroxy-5-isopropoxyquinazoline di-trifluoroacetic acid salt (49 g), 1,2-dichloroethane (440 ml) and DMF (245 ml) and the mixture was stirred and heated to 90° C. for 3.5 hours. An additional portion (7 g) of potassium carbonate was added and the mixture was stirred at 90° C. for a further hour. The reaction mixture was cooled to ambient temperature and the solids were filtered off and washed with methylene chloride. The filtrate and washings were combined and evaporated. The resultant residue was purified by column chromatography on silica using increasingly polar mixtures of methylene chloride and methanol (from a 50:1 mixture to a 20:1 mixture) as eluent. There was thus obtained 7-(2-chloroethoxy)-4-(5-chloro-2,3-methylenedioxypyrid-4-ylamino)-5-isopropoxyquinazoline as a white solid (37.1 g); Mass Spectrum: M+H⁺ 437 and 439.

1. The use of an anti-angiogenic agent in combination with an inhibitor of the Src family of non-receptor tyrosine kinases in the manufacture of a medicament for use in the substantially normotensive treatment in a warm-blooded mammal such as a human being of a disease state associated with angiogenesis, the Src kinase inhibitor being administered in an amount effective to counteract substantially the hypertension induced by the anti-angiogenic agent.

2. The use of an anti-angiogenic agent in combination with a Src kinase inhibitor according to claim 1 in the manufacture of a medicament for use in the substantially normotensive production of an anti-cancer effect in a warm-blooded mammal such as a human being, the Src kinase inhibitor being administered in an amount effective to counteract substantially the hypertension induced by the anti-angiogenic agent.

3. The use according to claim 1 of an anti-angiogenic agent in combination with a Src kinase inhibitor wherein the anti-angiogenic agent is selected from:
4-(4-fluoro-2-methylindol-5-yloxy)-6-methoxy-7-(3-(pyrrolidin-1-yl)propoxy)quinazoline,
4-(4-fluoro-2-methylindol-5-yloxy)-6-methoxy-7-(3-piperidinopropoxy)quinazoline,
4-(4-fluoro-2-methylindol-5-yloxy)-6-methoxy-7-((1-methylpiperidin-4-yl)methoxy)quinazoline,
4-(4-fluoro-2-methylindol-5-yloxy)-6-methoxy-7-(3-(4-methylpiperazin-1-yl)propoxy)quinazoline,
4-(4-fluoro-2-methylindol-5-yloxy)-6-methoxy-7-(2-(1-methylpiperidin-4-yl)ethoxy)quinazoline,
4-(4-chloro-2-fluoroanilino)-6-methoxy-7-(1-methylpiperidin-4-ylmethoxy)quinazoline,
4-(4-bromo-2-fluoroanilino)-6-methoxy-7-(1-methylpiperidin-4-ylmethoxy)quinazoline,
4-(4chloro-2-fluoroanilino)-6-methoxy-7-(piperidin-4-ylmethoxy)quinazoline,
4-(4-bromo-2-fluoroanilino)-6-methoxy-7-(piperidin-4-ylmethoxy)quinazoline,
4-[(4-fluoro-2-methyl-1H-indol-5-yl)oxy)-6-methoxy-7-{3-[4-(2,2,2-trifluoroethyl)piperazin-1-yl]propoxy}quinazoline,
7-{2-[4-(2-fluoroethyl)piperazin-1-yl]ethoxy]}[(4-fluoro-2-methyl-1H-indol-5-yl)oxy]-6-methoxyquinazoline,
4-[(4-fluoro-2-methylindol-5-yl)oxy]-6-methoxy-7-{3-[4-(2-propynyl)piperazin-1-yl]propoxy}quinazoline,
7-{3-[4-(2-fluoroethyl)piperazin-1-yl]propoxy}-4-[(4-fluoro-2-methyl-1H-indol-5-yl)oxy]-6-methoxyquinazoline,
7-(3-(4-acetylpiperazin-1-yl)propoxy]-4-(4-fluoro-2-methylindol-5-yloxy)-6-methoxyquinazoline, and
7-[2-(4-acetylpiperazin-1-yl)ethoxy]-4-[(4-fluoro-2-methyl-1H-indol-5-yl)oxy]-6-methoxyquinazoline;
or a pharmaceutically-acceptable acid-addition salt thereof.

4. The use according to claim 1 of an anti-angiogenic agent in combination with a Src kinase inhibitor wherein the Src kinase inhibitor selected from:
4-(2,4-dichloro-5-yl-methoxyanilino)-7-(2-piperidinoethoxy)-5-tetrahydropyran-4-yloxyquinazoline,
4-(2,4-dichloro-5-methoxyanilino)-7-(2-morpholinoethoxy)-5-tetrahydropyran-4-yloxyquinazoline,
4-(6-chloro-2,3-methylenedioxyanilino)-7-(2-pyrrolidin-1-ylethoxy)-5-tetrahydropyran-4-yloxyquinazoline,
4-(6-chloro-2,3-methylenedioxyanilino)-7-(3-pyrrolidin-1-ylpropoxy)-5-tetrahydropyran-4-yloxyquinazoline,
4-(6-chloro-2,3-methylenedioxyanilino)-7-[2-(4-methylpiperazin-1-yl)ethoxy]-5-tetrahydropyran-4-yloxyquinazoline,
4-(6-chloro-2,3-methylenedioxyanilino)-7-(2-piperidinoethoxy)-5-tetrahydropyran-4-yloxyquinazoline,
7-[2-(4-acetylpiperazin-1-yl)ethoxy]-4-(6-chloro-2,3-methylenedioxyanilino)-5-isopropoxyquinazoline,
6-methoxy-4-(2,3-methylenedioxyanilino)-7-(3-morpholinopropoxy)quinazoline,
6-methoxy-4-(2,3-methylenedioxyanilino)-7-(3-pyrrolidin-1-ylpropoxy)quinazoline,
6-methoxy-4-(2,3-methylenedioxyanilino)-7-[3-(4-methylpiperazin-1-yl)propoxy]quinazoline,
6-methoxy-4-(2,3-methylenedioxyanilino)-7-(3-piperidinopropoxy)quinazoline,
4-(6-chloro-2,3-methylenedioxyanilino)-7-[3-(4-isobutyrylpiperazine-1-yl)propoxy]-6-methoxyquinazoline,
4-(2-chloro-5-methoxyanilino)-6-methoxy-7-(N-Methylpiperidin-4-ylmethoxy)quinazoline,
4-(2-chloro-5-methoxyanilino)-6-methoxy-7-piperidin-4-ylmethoxyquinazoline,
4-(2,4-dichloro-5-methoxyanilino)-6-methoxy-7-(N-Methylpiperidin-4-ylmethoxy)quinazoline,
4-(2,4-dichloro-5-methoxyanilino)-6-methoxy-7-piperidin-4-ylmethoxyquinazoline,
7-[2-(4-acetylpiperazin-1-yl)ethoxy]-4-(5-chloro-2,3-methylenedioxypyrid-4-ylamino)-5-tetrahydropyran-4-yloxyquinazoline,
4-(5-chloro-2,3-methylenedioxypyrid-4-ylamino)-7-{2-[(3RS,4SR)-3,4-methylenedioxypyrrolidin-1-yl)ethoxy]-5-tetrahydropyran-4-yloxyquinazoline,
7-[2-(4-acetylpiperazin-1-yl)ethoxy]-4-(5-chloro-2,3-methylenedioxypyrid-4-ylamino)-5-isopropoxyquinazoline and
4-(5-chloro-2,3-methylenedioxypyrid-4-ylamino)-7-{2-[(3RS,4SR)-3,4-methylenedioxypyrrolidin-1-yl)ethoxy]-5-isopropoxyquinazoline;
or a pharmaceutically-acceptable acid-addition salt thereof.

5. A synergistic combination product comprising an anti-angiogenic agent and a Src kinase inhibitor for use simultaneously, sequentially or separately in the production of an anti-cancer effect in a warm-blooded animal such as a human being.

6. A synergistic combination product according to claim 5 wherein the anti-angiogenic agent is selected from the compounds listed in claim 3 and the Src kinase inhibitor is selected from the compounds listed in claim 4.

7. A blood pressure effect sparing combination product comprising an anti-angiogenic agent and a Src kinase inhibitor for use simultaneously, sequentially or separately in the production of an anti-cancer effect in a warm-blooded animal such as a human being.

8. A blood pressure effect sparing combination according to claim 7 wherein the anti-angiogenic agent is selected from the compounds listed in claim 3 and the Src kinase inhibitor is selected from the compounds listed in claim 4.

9. A combination product comprising an anti-angiogenic agent and a Src kinase inhibitor for use simultaneously, sequentially or separately in the production of a substantially normotensive anti-cancer effect in a warm-blooded mammal such as a human being.

10. A combination product according to claim 9 wherein the an anti-angiogenic agent is selected from:
4-(4-fluoro-2-methylindol-5-yloxy)-6-methoxy-7-(3-(pyrrolidin-1-yl)propoxy)quinazoline,
4-(4-fluoro-2-methylindol-5-yloxy)-6-methoxy-7-(3-piperidinopropoxy)quinazoline,
4-(4-fluoro-2-methylindol-5-yloxy)-6-methoxy-7-(3-(4-methylpiperazin-1-yl)propoxy)quinazoline,
4-(4-fluoro-2-methylindol-5-yloxy)-6-methoxy-7-(2-(1-methylpiperidin-4-yl)ethoxy)quinazoline,
4-(4-chloro-2-fluoroanilino)-6-methoxy-7-(1-methylpiperidin-4-ylmethoxy)quinazoline,
4-(4-bromo-2-fluoroanilino)-6-methoxy-7-(1-methylpiperidin-4-ylmethoxy)quinazoline,
4-(4-bromo-2-fluoroanilino)-6-methoxy-7-(piperidinylmethoxy)quinazoline,
4-[(4-fluoro-2-methylindol-5-yl)oxy]-6-methoxy-7-{3-[4-(2-propynyl)piperazin-1-yl]propoxy}quinazoline,
7-(3-(4-acetylpiperazin-1-yl)propoxy)-4-(4-fluoro-2-methylindol-5-yloxy)-6-methoxyquinazoline and
7-[2-(4-acetylpiperazin-1-yl)ethoxy]4-[(4-fluoro-2-methyl-1H-indol-5-yl)oxy]-6-methoxyquinazoline,
or a pharmaceutically-acceptable acid-addition salt thereof;
and the Src kinase inhibitor is selected from:
4-(6-chloro-2,3-methylenedioxyanilino)-7-(2-pyrrolidin-1-ylethoxy)-5-tetrahydropyran-4-yloxyquinazoline,
4-(6-chloro-2,3-methylenedioxyanilino)-7-(3-pyrrolidin-1-ylpropoxy)-5-tetrahydropyran-4-yloxyquinazoline,
4-(6-chloro-2,3-methylenedioxyanilino)-7-[2-(4-methylpiperazin-1-yl)ethoxy]-5-tetrahydropyran-4-yloxyquinazoline,
4-(6-chloro-2,3-methylenedioxyanilino)-7-(2-piperidinoethoxy)-5-tetrahydropyran-4-yloxyquinazoline,
7-[2-(4-acetylpiperazin-1-yl)ethoxy]-4-(6-chloro-2,3-methylenedioxyanilino)-5-isopropoxyquinazoline,
6-methoxy-4-(2,3-methylenedioxyanilino)-7-(3-morpholinopropoxy)quinazoline,
6-methoxy-4-(2,3-methylenedioxyanilino)-7-(3-piperidinopropoxy)quinazoline,
4-(6-chloro-2,3-methylenedioxyanilino)-7-[3-(4-isobutyrylpiperazine-1-yl)propoxy]-6-methoxyquinazoline,
4-(2-chloro-5-methoxyanilino)-6-methoxy-7-(N-Methylpiperidin-4-ylmethoxy)quinazoline,
7-[2-(4-acetylpiperazin-1-yl)ethoxy]-4-(5-chloro-2,3-methylenedioxypyrid-4-ylamino)-5-tetrahydropyran-4-yloxyquinazoline,
4-(5-chloro-2,3-methylenedioxypyrid-4-ylamino)-7-{2-[(3RS,4SR)-3,4-methylenedioxypyrrolidin-1-yl)ethoxy]-5-tetrahydropyran-4-yloxyquinazoline,
7-[2-(4-acetylpiperazin-1-yl)ethoxy]-4-(5-chloro-2,3-methylenedioxypyrid-4-ylamino)-5-isopropoxyquinazoline and
4-(5-chloro-2,3-methylenedioxypyrid-4-ylamino)-7-{2-[(3RS,4SR)-3,4-methylenedioxypyrrolidin-1-yl)ethoxy]-5-isopropoxyquinazoline,
or a pharmaceutically-acceptable acid-addition salt thereof.

11. A pharmaceutical composition comprising a combination product according to any of claims 5 to 10 and a pharmaceutically-acceptable excipient or carrier.

12. A combination product according to any of claims 5 to 10 wherein the anti-angiogenic agent is 4-(4-fluoro-2-methylindol-5-yloxy)-6-methoxy-7-(3-piperidinopropoxy)quinazoline, or a pharmaceutically-acceptable acid-addition salt thereof, and the Src kinase inhibitor is 7-[2-(4-acetylpiperazin-1-yl)ethoxy]-4-(6-chloro-2,3-methylenedioxyanilino)-5-isopropoxyquinazoline, or a pharmaceutically-acceptable acid-addition salt thereof.
